(19) United States
(12) Patent Application Publication
(10) Pub. No.: US 2025/0313532 A1
(43) Pub. Date: Oct. 9, 2025

(54) ALDH2 INHIBITORS AND METHODS OF USE THEREOF

(71) Applicant: UNIVERSITY OF MARYLAND, BALTIMORE, Baltimore, MD (US)

(72) Inventors: Fengtian Xue, Potomac, MD (US); Yong Ai, Halethorpe, MD (US)

(21) Appl. No.: 18/867,784

(22) PCT Filed: May 31, 2023
(86) PCT No.: PCT/US2023/067695

§ 371 Date: Nov. 20, 2024

Related U.S. Application Data
(60) Provisional application No. 63/347,518, filed on May 31, 2022.

(51) Int. Cl. C07D 215/20; A61K 31/37; A61K 31/429; A61K 31/4433; A61K 31/444; A61K 31/47; A61K 31/497; A61P 25/32; C07D 215/38; C07D 311/16; C07D 405/04; C07D 405/12; C07D 405/14; C07D 513/04

(52) U.S. Cl. CPC C07D 215/20; A61K 31/37; A61K 31/429; A61K 31/4433; A61K 31/444; A61K 31/47; A61K 31/497; A61P 25/32; C07D 215/38; C07D 311/16; C07D 405/04; C07D 405/12; C07D 405/14; C07D 513/04

(57) ABSTRACT

Substituted chromen-2-one compounds, synthesized using computer-aided virtual screening in combination with structure-based drug design (SBDD), for inhibiting the enzymatic activity of alcohol dehydrogenase-2 (ALDH2) and liver-specific OATP1-dependent uptake, pharmaceutical compositions thereof, and methods of using the same for the treatment of alcohol use disorders (AUDs) are disclosed. In some embodiments, the inventive compounds inhibit liver-specific ALDH2 protein.

### CROSS-REFERENCE TO RELATED APPLICATIONS

This application claims the benefit of U.S. Provisional Patent Application No. 63/347,518, filed May 31, 2022, which is incorporated by reference herein in its entirety.

### FIELD

The disclosure relates generally to compounds and methods of treating conditions using a multi-targeting strategy of inhibiting enzymatic activity of ALDH2 and liver-specific OATP1-dependent uptake.

### BACKGROUND

Alcohol use disorders (AUDs) represent a leading health issue that causes an enormous number of deaths and disabilities globally. Individuals with AUDs commonly fail in controlling drinking due to their alcohol dependence, leading to end-stage organ failure, such as alcoholic cirrhosis. Therapeutic prevention of AUDs remains limited and ineffective. NIAAA emphasizes the development of effective prevention and treatment strategies that can address the risks resulting from excessive drinking as its high research priority.

There is a need in the art for novel treatments for Alcohol use disorders.

### SUMMARY

In one aspect, the disclosure provides a compound of Formula (I), or a pharmaceutically acceptable salt, solvate, hydrate, cocrystal, or prodrug thereof:

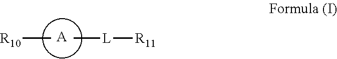

wherein
\textcircled{A} is optionally substituted heteroaryl;
R₁₀ is selected from halo, optionally substituted aryl, optionally substituted heteroaryl;
R₁₁ is selected from —OH, —C(═O)H, —C(═O)OH, —C(═O)OR₁₃, and —C(═O)NH₂;
L is a linker;
R₁₃ is selected from optionally substituted alkyl and optionally substituted alkenyl.

In some embodiments, L is a bond or —O—(CH₂)ₙ—R₁₄—;
wherein R₁₄ is selected from optionally substituted cycloalkyl, optionally substituted heterocyclyl, optionally substituted aryl, and optionally substituted heteroaryl; and
n is an integer selected from 1, 2, 3, 4, 5, or 6.

In some embodiments, R₁₀ is F, Br, Cl, I,

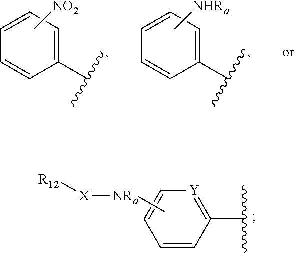

X is selected from —C(═O)— and —S(═O)₂—
Y is selected from CH and N;
R₁₂ is selected from optionally substituted alkyl, optionally substituted cycloalkyl, optionally substituted heterocyclyl, optionally substituted aryl, and optionally substituted heteroaryl; and
Rₐ is selected from H and optionally substituted alkyl.

In some embodiments, X is selected from —C(═O)— and —S(═O)₂—
Y is selected from CH and N;
R₁₂ is selected from optionally substituted alkyl, optionally substituted cycloalkyl, optionally substituted heterocyclyl, optionally substituted aryl, and optionally substituted heteroaryl; and
Rₐ is selected from H and optionally substituted alkyl.

In some embodiments, the compound is selected from Formula 1001 to Formula 1008, or a pharmaceutically acceptable salt, solvate, hydrate, cocrystal, or prodrug thereof.

In some embodiments, the compound of Formula (I) is a compound of formula 1007:

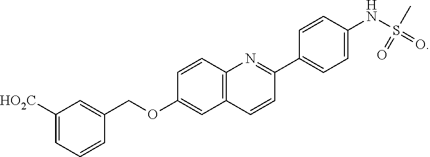 [SMILES: CS(=O)(=O)Nc1ccc(-c2ccc3cc(OCc4cccc(C(=O)O)c4)ccc3n2)cc1]

In some embodiments, \textcircled{A} is

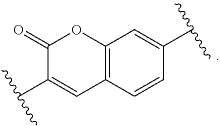 [SMILES: *c1ccc2cc(*)c(=O)oc2c1]

In some embodiments, the compound of Formula (I) is a compound of Formula (IIa) Formula (IIb), Formula (IIc), or a pharmaceutically acceptable salt, solvate, hydrate, cocrystal, or prodrug thereof:

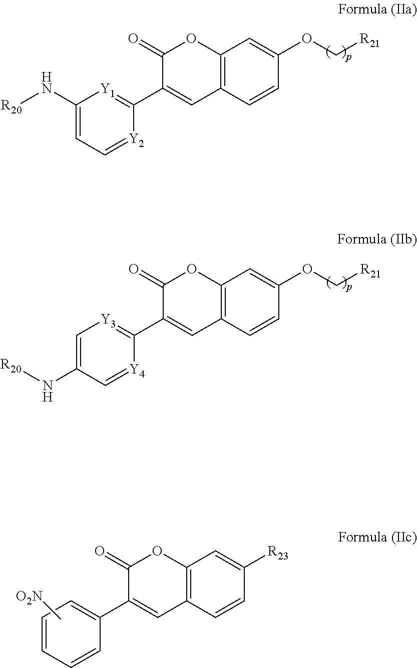

wherein
Y₁ and Y₂ are each CH, Y₁ is CH and Y₂ is N, or Y₁ is N and Y₂ is CH;
Y₃ and Y₄ are each CH, Y₃ is CH and Y₄ is N, or Y₃ is N and Y₄ is CH;
R₂₀ is selected from H, —S(═O)₂R₂₄, and —C(═O)₂R₂₄;
R₂₁ is selected from —C(O)OH, optionally substituted aryl, optionally substituted heteroaryl, and optionally substituted cycloalkyl;
R₂₃ is selected from —OH and

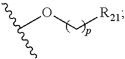

and
R₂₄ is selected from optionally substituted alkyl and optionally substituted cycloalkyl; and
p is an integer selected from 1 to 14.

In some embodiments, the compound of Formula (IIb) is a compound of Formula (20), or a pharmaceutically acceptable salt, solvate, hydrate, cocrystal, or prodrug thereof:

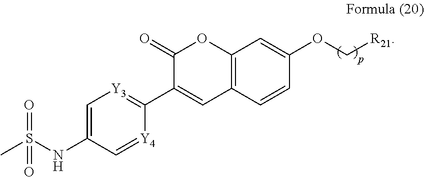

In some embodiments, R₂₄ is selected from methyl and cyclopropyl. In some embodiments, R₂₀ is selected from H, —S(═O)₂CH₃, —C(═O)CH₃, and

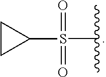 [SMILES: *S(=O)(=O)C1CC1]

In some embodiments, R₂₁ is selected from optionally substituted phenyl, optionally substituted cyclohexyl, optionally substituted pyridine, and optionally substituted pyrazine. In some embodiments, R₂₁ is selected from

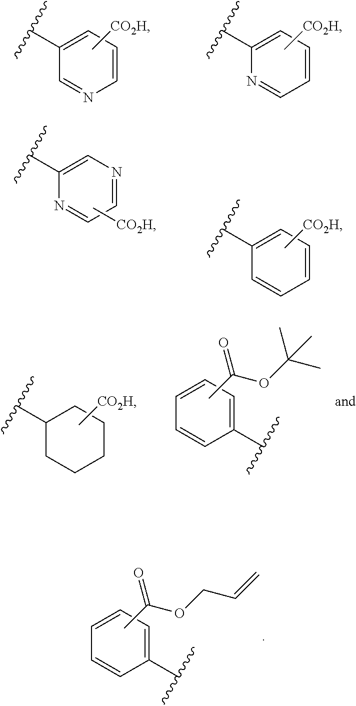

In some embodiments, p is 1. In some embodiments, Y₁ and Y₂ are each CH. In some embodiments, Y₃ and Y₄ are each CH. In some embodiments, Y₃ is N and Y₄ is CH.

In some embodiments, the compound of Formula (IIb) is a compound of Formula (21), or a pharmaceutically acceptable salt, solvate, hydrate, cocrystal, or prodrug thereof:

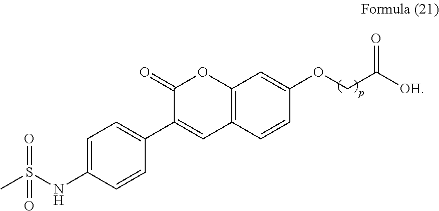

In some embodiments, in Formula (21), p is an integer from 4-14.

In some embodiments, the compound is selected from Formula 2001 to Formula 2131, or a pharmaceutically acceptable salt, solvate, hydrate, cocrystal, or prodrug thereof.

In some embodiments, the compound of Formula (I) is a compound of formula 2046, or a pharmaceutically acceptable salt, solvate, hydrate, cocrystal, or prodrug thereof:

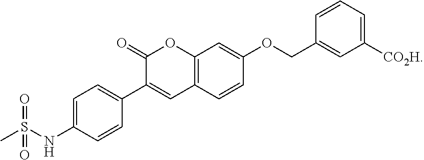 [SMILES: CS(=O)(=O)Nc1ccc(-c2cc3ccc(OCc4cccc(C(=O)O)c4)cc3oc2=O)cc1]

In some embodiments, \textcircled{A} is

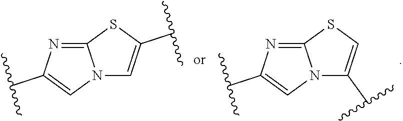 [SMILES: *c1cn2cc(*)sc2n1 ; *c1cn2c(*)csc2n1]

In some embodiments, wherein the compound of Formula (I) is a compound of Formula (IIIa) Formula (IIIb), Formula (IIIc), or a pharmaceutically acceptable salt, solvate, hydrate, cocrystal, or prodrug thereof:

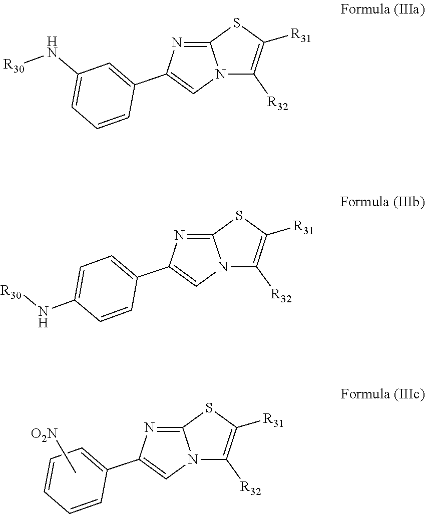

wherein
R₃₀ is selected from H, —S(═O)₂R₃₄, and —C(═O)₂R₃₄;
R₃₁ and R₃₂ are each independently selected from H, alkyl, —OH, —C(═O)H, —C(═O)OH, —C(═O)OR₃₃, and —C(═O)NH₂;
R₃₃ is alkyl or alkenyl; and
R₃₄ is selected from optionally substituted alkyl and optionally substituted cycloalkyl.

In some embodiments, R₃₀ is selected from H, —S(═O)₂CH₃, —C(═O)CH₃, and

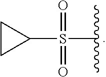 [SMILES: *S(=O)(=O)C1CC1]

In some embodiments, one of R₃₁ or R₃₂ is —OH, —C(═O)H, —C(═O)OH, —C(═O)OR₃₃, and —C(═O)NH₂

In some embodiments, the compound is selected from Formula 3001 to Formula 3073, or a pharmaceutically acceptable salt, solvate, hydrate, cocrystal, or prodrug thereof.

In some embodiments, the compound of Formula (I) is a compound of formula 3035, or a pharmaceutically acceptable salt, solvate, hydrate, cocrystal, or prodrug thereof:

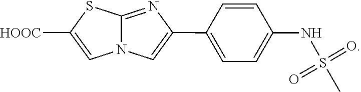

In some embodiments, the compound of Formula (I) is a compound of formula 3037, or a pharmaceutically acceptable salt, solvate, hydrate, cocrystal, or prodrug thereof:

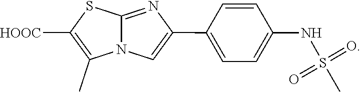

In some embodiments, the compound of any one of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073 inhibits alcohol dehydrogenase-2 (ALDH2) protein. In some embodiments, the compound of any one of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073 inhibits liver-specific alcohol dehydrogenase-2 (ALDH2) protein.

In one aspect, the disclosure provides a pharmaceutical composition comprising a compound of any one of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073 or a pharmaceutically acceptable salt, solvate, hydrate, cocrystal, or prodrug thereof, and a pharmaceutically acceptable carrier or excipient. In one aspect, the disclosure provides method of treating a condition by inhibiting alcohol dehydrogenase-2 (ALDH2) protein activity in a patient in need of said treatment, the method comprising administering to the patient a therapeutically effective amount of a compound of any one of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, or a pharmaceutically acceptable salt, solvate, hydrate, cocrystal, or prodrug thereof, or a therapeutically effective amount of the pharmaceutical composition. In some embodiments, the compound inhibits liver-specific ALDH2 protein. In some embodiments, the condition is an alcohol use disorder.

### BRIEF DESCRIPTION OF THE FIGURES

FIG. 1 illustrates CADD SILCS characterization of ALDH2 (PDB ID: 5L13). The apolar (green), H-bond-donor (blue), H-bond-acceptor (red), negative (orange), and positive (cyan) FragMaps are shown. FragMaps are shown at contour levels of −1.0 kcal/mol for the generic apolar (green) Hbond-donor (blue) and Hbond acceptor (red) maps and at −1.5 kcal/mol for the negative (orange), and positive (cyan) maps. The binding mode of an example hit CB-01 is shown in yellow.

FIG. 2 illustrates a table showing examples of virtual screening hits. *4DBA is a CADD parameter considering molecular weight, numbers of H-bond donor/acceptor, and Log P values of the compounds.

FIG. 3A illustrates an exemplary compound design strategy. FX01-FX02 are novel chemotypes and YA7241 and YA7068 are exemplary inhibitors. The sulfonamide head is shown in red; the heterocyclic core is shown in blue, and the liver-targeting carboxylic acid is shown in pink. FIG. 3B is a graph of experimental data illustrating inhibitory dose-response curves of new inhibitors CB-01, YA7241 and YA7068 in ALDH2 enzymatic inhibition assay. FIG. 3C illustrates IC₅₀ values of new compounds in comparison with the positive control disulfiram.

FIG. 3D is a graph of experimental data illustrating uptake of YA7068 by HEK293 vs OATP1B1-HEK293 cells.

FIG. 4 illustrates an image of the binding mode of YA7068 (yellow) in ALDH2 (PDB ID 5L13) by CADD SILCS docking analysis. The cartoon view of ALDH2 monomer in green. The residues that directly interact with the ligand are shown in orange. YA7068 is shown in yellow.

FIG. 5 illustrates an exemplary strategy for the development of compounds of the disclosure.

FIG. 6A illustrates an exemplary design of new metal-salophen complexes (top) and the synthesis of new ALDH2 inhibitors I1-I36 (bottom) of the disclosure. FIG. 6B illustrates exemplary functional groups representing the R and R′ fragments. FIG. 6C illustrates SILCS based LGFE energy differences (kcal/mol) versus compound YA7068.

FIGS. 7A-7B illustrates screening results. FIG. 7A illustrates the overall screening statistics from the ALDH2 enzymatic activity pilot HTS from the Selleck Bioactive collection pilot screening. FIG. 7B illustrates a dose response curve and calculated IC₅₀ value of one example hit FX03 from the Selleck Bioactive collection pilot screening.

FIG. 8 illustrates exemplary ALDH2 inhibitors of the disclosure.

FIG. 9 illustrates experimental data demonstrating cytotoxicity studies of YA7068 to primary human hepatocytes.

FIG. 10 illustrates a non-limiting example of the synthesis of compound YA7068.

FIG. 11 illustrates a non-limiting example of the synthesis of compound YA7263.

FIG. 12 illustrates a non-limiting example of the synthesis of imidazo[2,1-b]thiazoles of the disclosure.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically compounds of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein. The terms “active pharmaceutical ingredient” and “drug” may also include those compounds described herein that inhibit ALDH2 and/or allow for OATP-mediated uptake.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include the ALDH2 enzyme and OATP1 transporters.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of Formula (I), Formula (IIa), Formula (IIb), Formula (IIc), Formula (20), Formula (21), Formula (IIa), Formula (IIb), Formula (IIc), Formulas 1001-1008, Formulas 2001-2131, or Formulas 3001-3073. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. IIIustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)—and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” or “heterocyclyl” refer to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed disclosure. All embodiments of the disclosure can, in the alternative, be more specifically defined by any of the transitional terms “comprising,” “consisting essentially of,” and “consisting of.”

### ALDH2 Inhibitors

In the human body, ingested alcohol is mainly metabolized in the liver (hepatocytes) by two enzymes, alcohol dehydrogenase (ADH) and aldehyde dehydrogenase-2 (ALDH2), which catalyze two sequential reactions to yield acetaldehyde and acetate, respectively. Approximately 8% of the world population, mainly of East Asian descent, harbor the ALDH2*2 allele that encodes an inactive mitochondrial isoenzyme, resulting in facial flushing and unpleasant feelings (eg, nausea, headaches, cardiac palpitations, and overall discomfort) caused by the accumulation of acetaldehyde after alcohol consumption. Thus, people with ALDH2-deficiency are at lower risks of developing AUDs, which suggests that inhibition of ALDH2 activity may be effective for the treatment of AUDs. Indeed, disulfiram (Antabuse™), an active-site targeting irreversible inhibitor of ALDH2, has been clinically used for the treatment of AUDs; however, its treatment is associated with many side effects including dangerous cardiac side effects (such as tachycardia, low blood pressure, and QTc prolongation, known as disulfiram ethanol reaction [DER]). Thus, there is an urgent need to develop more specific ALDH2 inhibitors for the treatment of AUDs with fewer side effects.

Alcohol use disorders (AUDs) are associated with high mortality and disease burdens. AUDs are among the most prevalent mental disorders affecting over 5% of the world's population. Heavy drinking is the most important risk factor for numerous liver problems (e.g., fatty liver, alcohol-associated hepatitis, fibrosis, and cirrhosis) and chronic pancreatitis. Overtime heavy drinking can also cause severe damages to the heart (e.g., cardiomyopathy, arrhythmias, stroke, and high blood pressure) and brain (e.g., Wernicke-Korsakoff Syndrome). Moreover, AUDs increase the incidence of many types of cancer. Furthermore, AUDs are a major contributing factor for head injuries, motor vehicle accidents, violence, and assaults. Thus, there are unmet medical needs for therapeutic strategies to target AUDs.

ALDH2 plays a critical role in ethanol metabolism. Ethanol metabolism in humans occurs mainly in the liver and is carried out by two enzymatic steps. The first step is catalyzed by alcohol dehydrogenase (ADH), and the second step is mainly catalyzed by mitochondrial enzyme ALDH2. ALDH2 is expressed ubiquitously in all tissues but most abundant in the liver. Among the 19 human ALDH isozymes, ALDH2 is the only member that contributes significantly to acetaldehyde metabolism. ALDH2 binds to acetaldehyde with a Km value of 0.2 μM, 900-fold lower than that of the abundant cytosolic isozyme ALDH1. Genetic polymorphism studies have shown that approximately 8% of the world's population, mostly found in East Asians, carries the ALDH2*2 allele that encodes for an inactive ALDH2. This deficiency in these individuals causes alcohol-induced flushing syndrome including facial flushing, headaches, nausea, dizziness, and cardiac palpitations. Compared to the WT ALDH2*1 allele, the ALDH2*2 individuals have a single G to A nucleotide change, leading to an E487K variant that impacts the dimer and tetramer formation of functional ALDH2. Heterozygotic (ALDH2*1/*2) and homozygotic (ALDH2*2/*2) individuals only possess <10% and <1-4% of the WT enzyme activity, respectively. Due to the unpleasant feelings caused by acetaldehyde accumulation following alcohol consumption, the ALDH2*2 carriers commonly indicate alcohol avoidance, resulting in a reduced prevalence of alcoholism.

Liver-specific inhibition of ALDH2 presents a promising treatment of AUDs. It was recently reported that the hepatocyte-specific Aldh2-deficient mice (Aldh2hep−/−) were resistant to alcohol-seeking behavior with elevated blood acetaldehyde levels. Interestingly, Aldh2hep−/− mice had much less reduced energy expenditure and motility than the global Aldh2 knockout mice. These data suggest that liver-specific inhibition of ALDH2 represents a highly promising therapeutic approach for the treatment of AUDs with fewer unwanted side effects.

ALDH2 is druggable yet druglike inhibitors are limited. The catalytic active site of ALDH2 includes three Cys residues (C301-C302-C303) different from the sequence found in other ALDH isozymes (eg, CCI for ALDH1A1). More interestingly, different from other isozymes (eg, ALDH1), ALDH2 has a smaller substrate entrance pocket. These structural differences provide opportunities for selective inhibition of ALDH2 versus other ALDH isozymes. Several small-molecule inhibitors of ALDH2 have been reported. These inhibitors achieved activities through either reversible binding to the active site or irreversible modification of the active site Cys residues. Although disulfiram has been approved to help AUD patients, it suffers from potentially lethal cardiac side effects. Daidzein and its derivative CVT10216 inhibit ALDH2 with good potency and isozyme selectivity. However, these compounds were based on a common isoflavone core, a scaffold well known for its estrogen-like effects. Besides, the clinical studies of ANS-6637 were discontinued due to major safety concerns. Importantly, all these known ALDH2 inhibitors block the global activity of ALDH2 with no liver-specificity.

Hepatocyte-selective uptake by the organic anion transporting polypeptides (OATPs). The OATPs are a group of 11 human multispecific transporters that are classified into six subfamilies based on their amino acid identity. Of all the OATPs, the OATP1 subfamily is best characterized, in particular, OATP1B1 and OATP1B3 are specifically expressed in the liver. It has been reported that the OATP1 subfamily are able to transport a variety and broad size range of small anionic molecules. For instance, the OATP1 transporters are responsible for the uptake of anionic acidic compounds such as bile acids, steroid hormones, and drugs (eg, enalapril and atorvastatin). The OATP1 subfamily of OATPs represents a major class of uptake transporters for liver-targeted drug development.

Recently, it has been reported that liver-specific gene deletion or shRNA inhibition of Aldh2 effectively reduced alcohol-seeking behavior, suggesting that liver-specific inhibition of ALDH2 represents a promising therapeutic approach for the treatment of AUDs with less unwanted side effects.

To develop liver-specific ALDH2 inhibitors, a novel coumarin-based small molecule YA7068 was synthesized using computer-aided virtual screening in combination with structure-based drug design (SBDD). Preliminary studies have shown YA7068 inhibits the enzymatic activity of ALDH2 with an excellent potency (IC₅₀=62 nM). Simultaneously, YA7068 functions as a substrate for the hepatocyte-specific organic anion transporting polypeptide 1B1 (OATP1B1), leading to liver-selective targeting of ALDH2. In addition, using the available ALDH2 crystal structures, the surface of the enzyme was characterized using a novel computer-aided drug design (CADD) method SILCS (site identification of ligand competitive saturation). The SILCS analyses revealed the presence of a “sweet spot” that maps to the substrate entrance pocket near the ALDH2 active site. These results suggest further optimization, through coupling compound activity at both sites, combined with an in-depth structure-activity relationship (SAR), will lead to the discovery of potent inhibitors for ALDH2, while retaining their OATP1B1 substrate activity. Here computer-aided drug design (CADD) is combined with medicinal chemistry, along with validated in vitro and in vivo assays to identify and characterize compounds that can be developed into potent and liver-specific inhibitors for ALDH2.

In one aspect, the disclosure provides compounds of Formula (I) or a pharmaceutically acceptable salt, solvate, hydrate, cocrystal, or prodrug thereof.

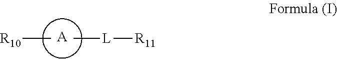

wherein
\textcircled{A} is optionally substituted heteroaryl;
R₁₀ is selected from halo, optionally substituted aryl, and optionally substituted heteroaryl;
R₁₁ is selected from —OH, —C(═O)H, —C(═O)OH, —C(═O)OR₁₃, and —C(═O)NH₂; L is a linker;
R₁₃ is selected from optionally substituted alkyl and optionally substituted alkenyl.

Any linker L is contemplated by the present disclosure. In some embodiments, L is a bond or —O—(CH₂)ₙ—R₁₄—;
wherein R₁₄ is selected from optionally substituted cycloalkyl, optionally substituted heterocyclyl, optionally substituted aryl, and optionally substituted heteroaryl; and n is an integer selected from 1, 2, 3, 4, 5, or 6.

In some embodiments, R₁₀ is F, Br, Cl, I,

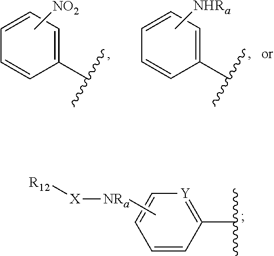

X is selected from —C(═O)— and —S(═O)₂—
Y is selected from CH and N;
R₁₂ is selected from optionally substituted alkyl, optionally substituted cycloalkyl, optionally substituted heterocyclyl, optionally substituted aryl, and optionally substituted heteroaryl; and
Rₐ is selected from H and optionally substituted alkyl.

Any optionally substituted heteroaryl ring is contemplated by the present disclosure for ring A, including but not limited to monocyclic and polycyclic rings. Non-limiting examples of ring A include coumarin, imidazthiazole, and quinoline.

In some embodiments, \textcircled{A} is

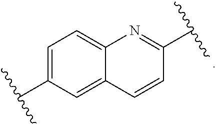 [SMILES: *c1ccc2nc(*)ccc2c1]

In some embodiments, L is —O—(CH₂)ₙ—R₁₄—;
R₁₀ is Cl or

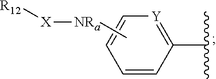

R₁₁ is selected from —C(═O)OH and —C(═O)OR₁₃;
R₁₂ is alkyl; and
R₁₄ is C₆ aryl.

In some embodiments, the compound is selected from:

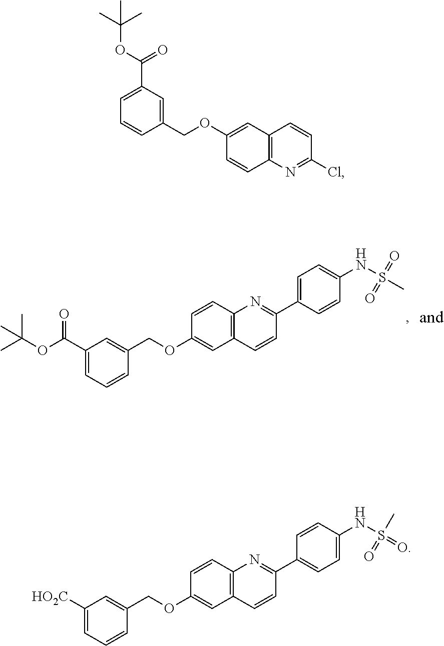 [SMILES: CC(C)(C)OC(=O)c1cccc(COc2ccc3nc(Cl)ccc3c2)c1 ; CC(C)(C)OC(=O)c1cccc(COc2ccc3nc(-c4ccc(NS(C)(=O)=O)cc4)ccc3c2)c1 ; CS(=O)(=O)Nc1ccc(-c2ccc3cc(OCc4cccc(C(=O)O)c4)ccc3n2)cc1]

In some embodiments, the compound of Formula (I) is a compound of formula 1007:

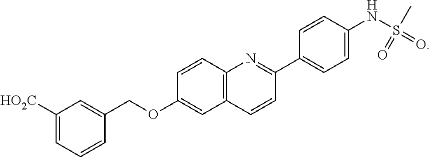 [SMILES: CS(=O)(=O)Nc1ccc(-c2ccc3cc(OCc4cccc(C(=O)O)c4)ccc3n2)cc1]

In some embodiments, \textcircled{A} is

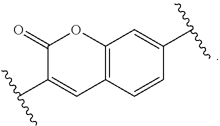 [SMILES: *c1ccc2cc(*)c(=O)oc2c1]

In some embodiments, the compound of Formula (I) is a compound of Formula (IIa) Formula (IIb), Formula (IIc), or a pharmaceutically acceptable salt, solvate, hydrate, cocrystal, or prodrug thereof:

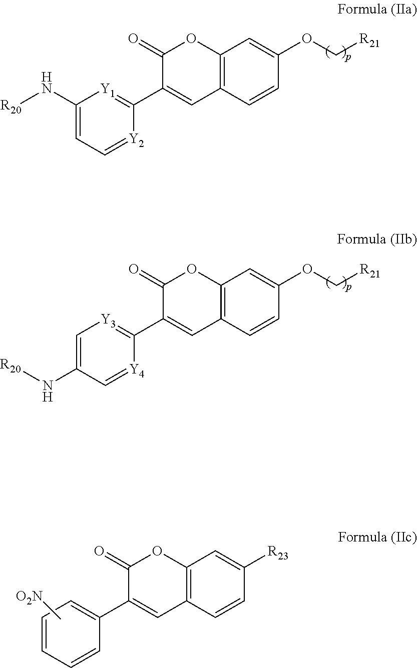

wherein
Y₁ and Y₂ are each CH, Y₁ is CH and Y₂ is N, or Y₁ is N and Y₂ is CH;
Y₃ and Y₄ are each CH, Y₃ is CH and Y₄ is N, or Y₃ is N and Y₄ is CH;
R₂₀ is selected from H, —S(═O)₂R₂₄, and —C(═O)₂R₂₄;
R₂₁ is selected from —C(O)OH, optionally substituted aryl, optionally substituted heteroaryl, and optionally substituted cycloalkyl;
R₂₃ is selected from —OH and

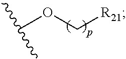

and
R₂₄ is selected from optionally substituted alkyl and optionally substituted cycloalkyl; and
p is an integer selected from 1-14.

In some embodiments, p is an integer from 1 to 6. In some embodiments, p is an integer from 4 to 14.

In some embodiments, the compound of Formula (IIb) is a compound of Formula (20), or a pharmaceutically acceptable salt, solvate, hydrate, cocrystal, or prodrug thereof:

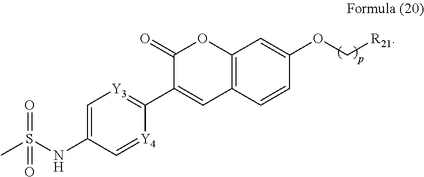

In some embodiments, the compound of Formula (IIb) is a compound of Formula (21), or a pharmaceutically acceptable salt, solvate, hydrate, cocrystal, or prodrug thereof:

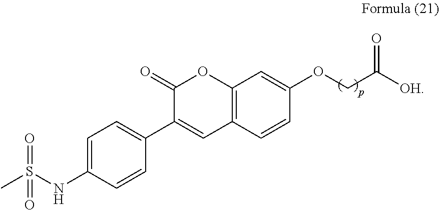

In some embodiments, in Formula (21), p is an integer from 4-14.

In some embodiments, R₂₄ is optionally substituted alkyl. In some embodiments, R₂₄ is optionally substituted C₁-C₆alkyl. In some embodiments, R₂₄ is methyl. In some embodiments, R₂₄ is optionally substituted cycloalkyl. In some embodiments, R₂₄ is optionally substituted C₃-C₆alky. In some embodiments, R₂₄ is cyclopropyl. In some embodiments, R₂₄ is selected from methyl and cyclopropyl.

In some embodiments, R₂₀ is selected from H, —S(═O)₂CH₃, —C(═O)CH₃, and

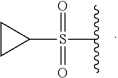 [SMILES: *S(=O)(=O)C1CC1]

Any optionally substituted aryl, optionally substituted heteroaryl, and optionally substituted cycloalkyl are contemplated by the present disclosure for R₂₁. In some embodiments, R₂₁ is selected from optionally substituted phenyl, optionally substituted cyclohexyl, optionally substituted pyridine, and optionally substituted pyrazine.

In some embodiments, R₂₁ is selected from

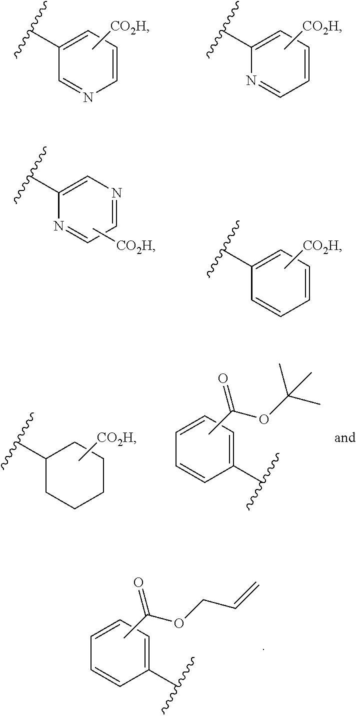

R₂₁ is selected from

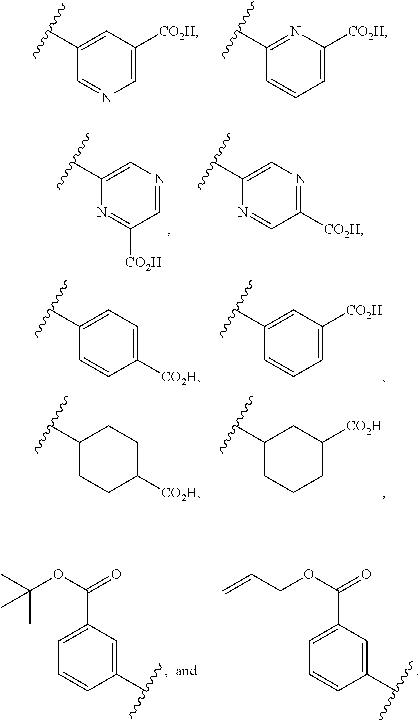

In some embodiments, p is 1.

In some embodiments, Y₁ and Y₂ are each CH.

In some embodiments, Y₃ and Y₄ are each CH.

In some embodiments, Y₃ is N and Y₄ is CH.

In some embodiments, the compound is of any one of formulas 2001 to 2131, or a pharmaceutically acceptable salt, solvate, hydrate, cocrystal, or prodrug thereof:

| | |
|---|---|
| | Formula (IIa) |
| 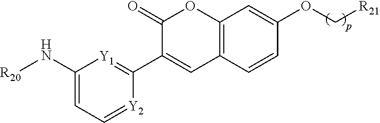 | |

| Compound # | R₂₀ | R₂₁ | Y₁ | Y₂ | p |
|---|---|---|---|---|---|
| 2001 | —S(═O)₂CH₃ | 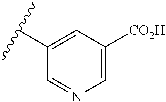 [SMILES: *c1cncc(C(=O)O)c1] | CH | CH | 1 |
| 2002 | —S(═O)₂CH₃ | 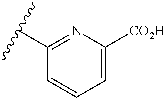 [SMILES: *c1cccc(C(=O)O)n1] | CH | CH | 1 |
| 2003 | —S(═O)₂CH₃ | 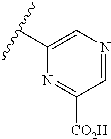 [SMILES: *c1cncc(C(=O)O)n1] | CH | CH | 1 |
| 2004 | —S(═O)₂CH₃ | 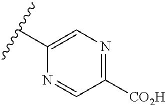 [SMILES: *c1cnc(C(=O)O)cn1] | CH | CH | 1 |
| 2005 | —S(═O)₂CH₃ | 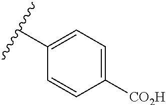 [SMILES: *c1ccc(C(=O)O)cc1] | CH | CH | 1 |
| 2006 | —S(═O)₂CH₃ | 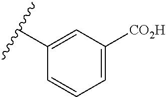 [SMILES: *c1cccc(C(=O)O)c1] | CH | CH | 1 |
| 2007 | —S(═O)₂CH₃ | 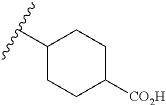 [SMILES: *C1CCC(C(=O)O)CC1] | CH | CH | 1 |
| 2008 | —S(═O)₂CH₃ | 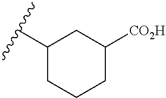 [SMILES: *C1CCCC(C(=O)O)C1] | CH | CH | 1 |
| 2009 | —S(═O)₂CH₃ | 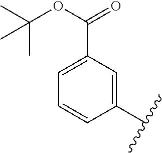 [SMILES: *c1cccc(C(=O)OC(C)(C)C)c1] | CH | CH | 1 |
| 2010 | —S(═O)₂CH₃ | 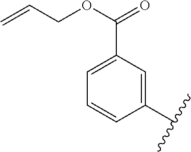 [SMILES: *c1cccc(C(=O)OCC=C)c1] | CH | CH | 1 |
| 2011 | —C(═O)CH₃ | 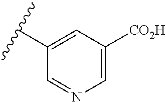 [SMILES: *c1cncc(C(=O)O)c1] | CH | CH | 1 |
| 2012 | —C(═O)CH₃ | 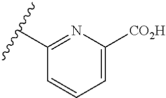 [SMILES: *c1cccc(C(=O)O)n1] | CH | CH | 1 |
| 2013 | —C(═O)CH₃ | 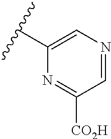 [SMILES: *c1cncc(C(=O)O)n1] | CH | CH | 1 |
| 2014 | —C(═O)CH₃ | 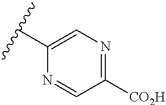 [SMILES: *c1cnc(C(=O)O)cn1] | CH | CH | 1 |
| 2015 | —C(═O)CH₃ | 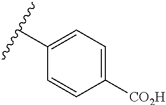 [SMILES: *c1ccc(C(=O)O)cc1] | CH | CH | 1 |
| 2016 | —C(═O)CH₃ | 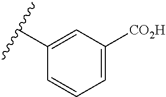 [SMILES: *c1cccc(C(=O)O)c1] | CH | CH | 1 |
| 2017 | —C(═O)CH₃ | 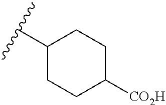 [SMILES: *C1CCC(C(=O)O)CC1] | CH | CH | 1 |
| 2018 | —C(═O)CH₃ | 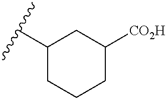 [SMILES: *C1CCCC(C(=O)O)C1] | CH | CH | 1 |
| 2019 | —C(═O)CH₃ | 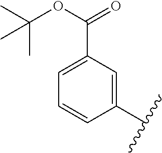 [SMILES: *c1cccc(C(=O)OC(C)(C)C)c1] | CH | CH | 1 |
| 2020 | —C(═O)CH₃ | 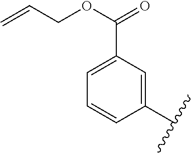 [SMILES: *c1cccc(C(=O)OCC=C)c1] | CH | CH | 1 |
| 2021 | 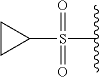 [SMILES: *S(=O)(=O)C1CC1] | 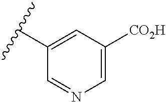 [SMILES: *c1cncc(C(=O)O)c1] | CH | CH | 1 |
| 2022 | 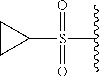 [SMILES: *S(=O)(=O)C1CC1] | 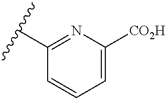 [SMILES: *c1cccc(C(=O)O)n1] | CH | CH | 1 |
| 2023 | 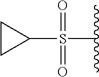 [SMILES: *S(=O)(=O)C1CC1] | 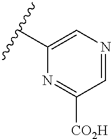 [SMILES: *c1cncc(C(=O)O)n1] | CH | CH | 1 |
| 2024 | 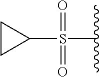 [SMILES: *S(=O)(=O)C1CC1] | 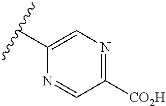 [SMILES: *c1cnc(C(=O)O)cn1] | CH | CH | 1 |
| 2025 | 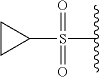 [SMILES: *S(=O)(=O)C1CC1] | 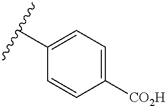 [SMILES: *c1ccc(C(=O)O)cc1] | CH | CH | 1 |
| 2026 | 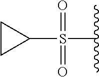 [SMILES: *S(=O)(=O)C1CC1] | 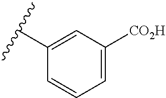 [SMILES: *c1cccc(C(=O)O)c1] | CH | CH | 1 |
| 2027 | 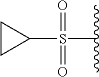 [SMILES: *S(=O)(=O)C1CC1] | 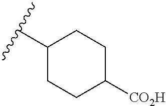 [SMILES: *C1CCC(C(=O)O)CC1] | CH | CH | 1 |
| 2028 | 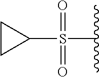 [SMILES: *S(=O)(=O)C1CC1] | 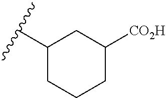 [SMILES: *C1CCCC(C(=O)O)C1] | CH | CH | 1 |
| 2029 | 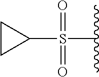 [SMILES: *S(=O)(=O)C1CC1] | 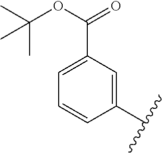 [SMILES: *c1cccc(C(=O)OC(C)(C)C)c1] | CH | CH | 1 |
| 2030 | 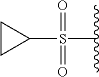 [SMILES: *S(=O)(=O)C1CC1] | 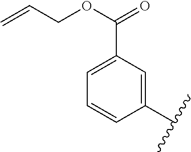 [SMILES: *c1cccc(C(=O)OCC=C)c1] | CH | CH | 1 |
| 2031 | H | 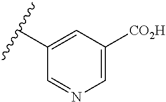 [SMILES: *c1cncc(C(=O)O)c1] | CH | CH | 1 |
| 2032 | H | 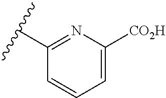 [SMILES: *c1cccc(C(=O)O)n1] | CH | CH | 1 |
| 2033 | H | 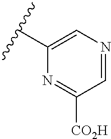 [SMILES: *c1cncc(C(=O)O)n1] | CH | CH | 1 |
| 2034 | H | 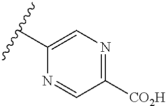 [SMILES: *c1cnc(C(=O)O)cn1] | CH | CH | 1 |
| 2035 | H | 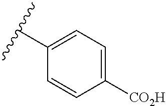 [SMILES: *c1ccc(C(=O)O)cc1] | CH | CH | 1 |
| 2036 | H | 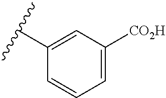 [SMILES: *c1cccc(C(=O)O)c1] | CH | CH | 1 |
| 2037 | H | 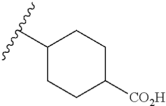 [SMILES: *C1CCC(C(=O)O)CC1] | CH | CH | 1 |
| 2038 | H | 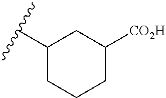 [SMILES: *C1CCCC(C(=O)O)C1] | CH | CH | 1 |
| 2039 | H | 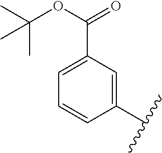 [SMILES: *c1cccc(C(=O)OC(C)(C)C)c1] | CH | CH | 1 |
| 2040 | H | 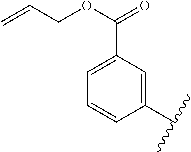 [SMILES: *c1cccc(C(=O)OCC=C)c1] | CH | CH | 1 |

| | |
|---|---|
| | Formula (IIb) |
| 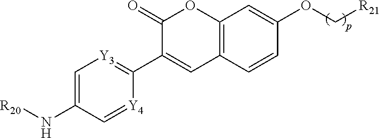 | |

| Compound # | R₂₀ | R₂₁ | Y₃ | Y₄ | p |
|---|---|---|---|---|---|
| 2041 | —S(═O)₂CH₃ | 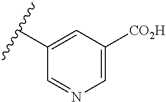 [SMILES: *c1cncc(C(=O)O)c1] | CH | CH | 1 |
| 2042 | —S(═O)₂CH₃ | 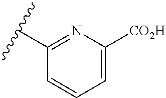 [SMILES: *c1cccc(C(=O)O)n1] | CH | CH | 1 |
| 2043 | —S(═O)₂CH₃ | 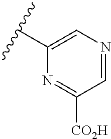 [SMILES: *c1cncc(C(=O)O)n1] | CH | CH | 1 |
| 2044 | —S(═O)₂CH₃ | 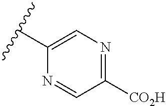 [SMILES: *c1cnc(C(=O)O)cn1] | CH | CH | 1 |
| 2045 | —S(═O)₂CH₃ | 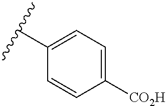 [SMILES: *c1ccc(C(=O)O)cc1] | CH | CH | 1 |
| 2046 | —S(═O)₂CH₃ | 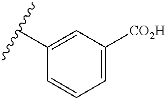 [SMILES: *c1cccc(C(=O)O)c1] | CH | CH | 1 |
| 2047 | —S(═O)₂CH₃ | 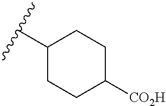 [SMILES: *C1CCC(C(=O)O)CC1] | CH | CH | 1 |
| 2048 | —S(═O)₂CH₃ | 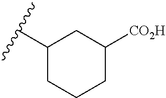 [SMILES: *C1CCCC(C(=O)O)C1] | CH | CH | 1 |
| 2049 | —S(═O)₂CH₃ | 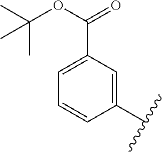 [SMILES: *c1cccc(C(=O)OC(C)(C)C)c1] | CH | CH | 1 |
| 2050 | —S(═O)₂CH₃ | 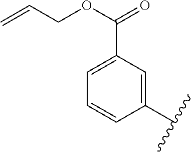 [SMILES: *c1cccc(C(=O)OCC=C)c1] | CH | CH | 1 |
| 2051 | —C(═O)CH₃ | 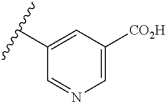 [SMILES: *c1cncc(C(=O)O)c1] | CH | CH | 1 |
| 2052 | —C(═O)CH₃ | 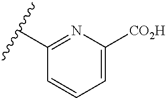 [SMILES: *c1cccc(C(=O)O)n1] | CH | CH | 1 |
| 2053 | —C(═O)CH₃ | 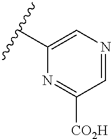 [SMILES: *c1cncc(C(=O)O)n1] | CH | CH | 1 |
| 2054 | —C(═O)CH₃ | 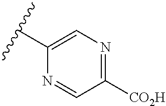 [SMILES: *c1cnc(C(=O)O)cn1] | CH | CH | 1 |
| 2055 | —C(═O)CH₃ | 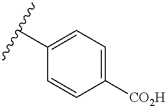 [SMILES: *c1ccc(C(=O)O)cc1] | CH | CH | 1 |
| 2056 | —C(═O)CH₃ | 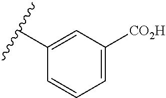 [SMILES: *c1cccc(C(=O)O)c1] | CH | CH | 1 |
| 2057 | —C(═O)CH₃ | 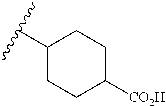 [SMILES: *C1CCC(C(=O)O)CC1] | CH | CH | 1 |
| 2058 | —C(═O)CH₃ | 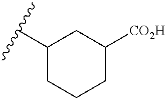 [SMILES: *C1CCCC(C(=O)O)C1] | CH | CH | 1 |
| 2059 | —C(═O)CH₃ | 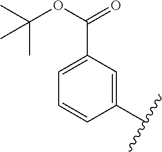 [SMILES: *c1cccc(C(=O)OC(C)(C)C)c1] | CH | CH | 1 |
| 2060 | —C(═O)CH₃ | 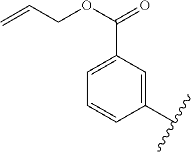 [SMILES: *c1cccc(C(=O)OCC=C)c1] | CH | CH | 1 |
| 2061 | 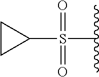 [SMILES: *S(=O)(=O)C1CC1] | 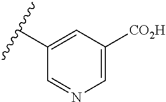 [SMILES: *c1cncc(C(=O)O)c1] | CH | CH | 1 |
| 2062 | 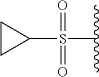 [SMILES: *S(=O)(=O)C1CC1] | 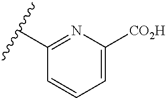 [SMILES: *c1cccc(C(=O)O)n1] | CH | CH | 1 |
| 2063 | 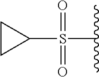 [SMILES: *S(=O)(=O)C1CC1] | 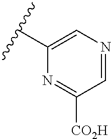 [SMILES: *c1cncc(C(=O)O)n1] | CH | CH | 1 |
| 2064 | 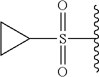 [SMILES: *S(=O)(=O)C1CC1] | 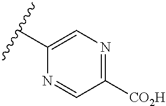 [SMILES: *c1cnc(C(=O)O)cn1] | CH | CH | 1 |
| 2065 | 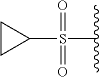 [SMILES: *S(=O)(=O)C1CC1] | 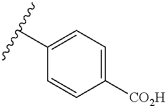 [SMILES: *c1ccc(C(=O)O)cc1] | CH | CH | 1 |
| 2066 | 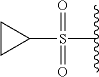 [SMILES: *S(=O)(=O)C1CC1] | 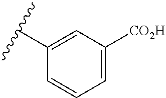 [SMILES: *c1cccc(C(=O)O)c1] | CH | CH | 1 |
| 2067 | 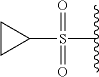 [SMILES: *S(=O)(=O)C1CC1] | 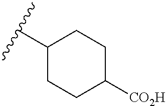 [SMILES: *C1CCC(C(=O)O)CC1] | CH | CH | 1 |
| 2068 | 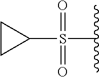 [SMILES: *S(=O)(=O)C1CC1] | 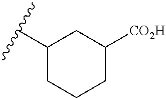 [SMILES: *C1CCCC(C(=O)O)C1] | CH | CH | 1 |
| 2069 | 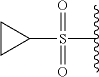 [SMILES: *S(=O)(=O)C1CC1] | 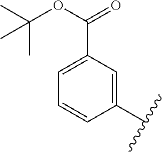 [SMILES: *c1cccc(C(=O)OC(C)(C)C)c1] | CH | CH | 1 |
| 2070 | 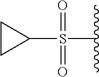 [SMILES: *S(=O)(=O)C1CC1] | 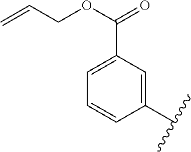 [SMILES: *c1cccc(C(=O)OCC=C)c1] | CH | CH | 1 |
| 2071 | H | 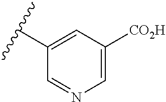 [SMILES: *c1cncc(C(=O)O)c1] | CH | CH | 1 |
| 2072 | H | 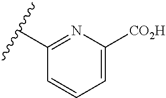 [SMILES: *c1cccc(C(=O)O)n1] | CH | CH | 1 |
| 2073 | H | 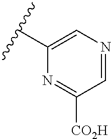 [SMILES: *c1cncc(C(=O)O)n1] | CH | CH | 1 |
| 2074 | H | 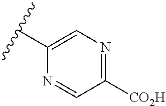 [SMILES: *c1cnc(C(=O)O)cn1] | CH | CH | 1 |
| 2075 | H | 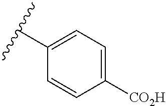 [SMILES: *c1ccc(C(=O)O)cc1] | CH | CH | 1 |
| 2076 | H | 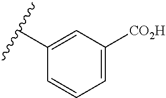 [SMILES: *c1cccc(C(=O)O)c1] | CH | CH | 1 |
| 2077 | H | 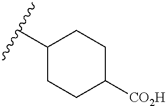 [SMILES: *C1CCC(C(=O)O)CC1] | CH | CH | 1 |
| 2078 | H | 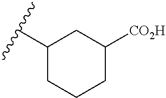 [SMILES: *C1CCCC(C(=O)O)C1] | CH | CH | 1 |
| 2079 | H | 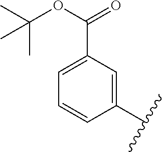 [SMILES: *c1cccc(C(=O)OC(C)(C)C)c1] | CH | CH | 1 |
| 2080 | H | 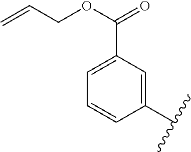 [SMILES: *c1cccc(C(=O)OCC=C)c1] | CH | CH | 1 |
| 2081 | —S(═O)₂CH₃ | 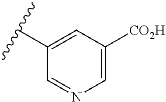 [SMILES: *c1cncc(C(=O)O)c1] | N | CH | 1 |
| 2082 | —S(═O)₂CH₃ | 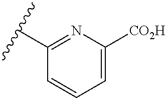 [SMILES: *c1cccc(C(=O)O)n1] | N | CH | 1 |
| 2083 | —S(═O)₂CH₃ | 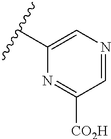 [SMILES: *c1cncc(C(=O)O)n1] | N | CH | 1 |
| 2084 | —S(═O)₂CH₃ | 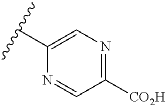 [SMILES: *c1cnc(C(=O)O)cn1] | N | CH | 1 |
| 2085 | —S(═O)₂CH₃ | 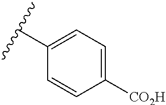 [SMILES: *c1ccc(C(=O)O)cc1] | N | CH | 1 |
| 2086 | —S(═O)₂CH₃ | 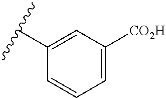 [SMILES: *c1cccc(C(=O)O)c1] | N | CH | 1 |
| 2087 | —S(═O)₂CH₃ | 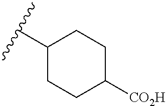 [SMILES: *C1CCC(C(=O)O)CC1] | N | CH | 1 |
| 2088 | —S(═O)₂CH₃ | 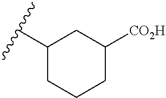 [SMILES: *C1CCCC(C(=O)O)C1] | N | CH | 1 |
| 2089 | —S(═O)₂CH₃ | 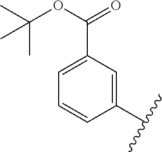 [SMILES: *c1cccc(C(=O)OC(C)(C)C)c1] | N | CH | 1 |
| 2090 | —S(═O)₂CH₃ | 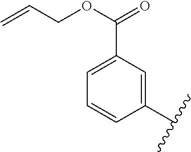 [SMILES: *c1cccc(C(=O)OCC=C)c1] | N | CH | 1 |
| 2091 | —C(═O)CH₃ | 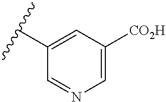 [SMILES: *c1cncc(C(=O)O)c1] | N | CH | 1 |
| 2092 | —C(═O)CH₃ | 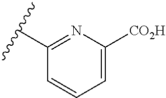 [SMILES: *c1cccc(C(=O)O)n1] | N | CH | 1 |
| 2093 | —C(═O)CH₃ | 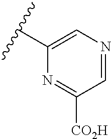 [SMILES: *c1cncc(C(=O)O)n1] | N | CH | 1 |
| 2094 | —C(═O)CH₃ | 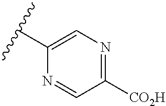 [SMILES: *c1cnc(C(=O)O)cn1] | N | CH | 1 |
| 2095 | —C(═O)CH₃ | 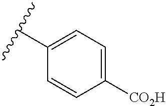 [SMILES: *c1ccc(C(=O)O)cc1] | N | CH | 1 |
| 2096 | —C(═O)CH₃ | 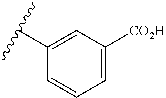 [SMILES: *c1cccc(C(=O)O)c1] | N | CH | 1 |
| 2097 | —C(═O)CH₃ | 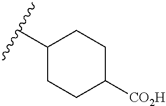 [SMILES: *C1CCC(C(=O)O)CC1] | N | CH | 1 |
| 2098 | —C(═O)CH₃ | 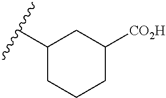 [SMILES: *C1CCCC(C(=O)O)C1] | N | CH | 1 |
| 2099 | —C(═O)CH₃ | 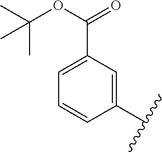 [SMILES: *c1cccc(C(=O)OC(C)(C)C)c1] | N | CH | 1 |
| 2100 | —C(═O)CH₃ | 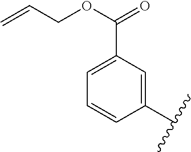 [SMILES: *c1cccc(C(=O)OCC=C)c1] | N | CH | 1 |
| 2101 | 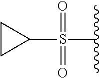 [SMILES: *S(=O)(=O)C1CC1] | 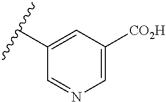 [SMILES: *c1cncc(C(=O)O)c1] | N | CH | 1 |
| 2102 | 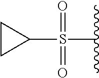 [SMILES: *S(=O)(=O)C1CC1] | 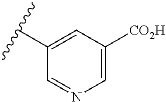 [SMILES: *c1cncc(C(=O)O)c1] | N | CH | 1 |
| 2103 | 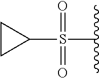 [SMILES: *S(=O)(=O)C1CC1] | 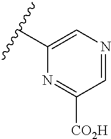 [SMILES: *c1cncc(C(=O)O)n1] | N | CH | 1 |
| 2104 | 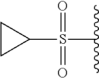 [SMILES: *S(=O)(=O)C1CC1] | 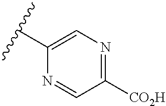 [SMILES: *c1cnc(C(=O)O)cn1] | N | CH | 1 |
| 2105 | 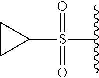 [SMILES: *S(=O)(=O)C1CC1] | 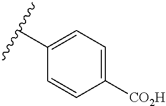 [SMILES: *c1ccc(C(=O)O)cc1] | N | CH | 1 |
| 2106 | 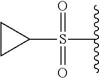 [SMILES: *S(=O)(=O)C1CC1] | 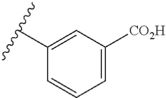 [SMILES: *c1cccc(C(=O)O)c1] | N | CH | 1 |
| 2107 | 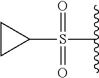 [SMILES: *S(=O)(=O)C1CC1] | 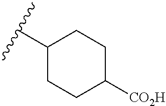 [SMILES: *C1CCC(C(=O)O)CC1] | N | CH | 1 |
| 2108 | 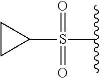 [SMILES: *S(=O)(=O)C1CC1] | 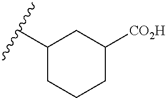 [SMILES: *C1CCCC(C(=O)O)C1] | N | CH | 1 |
| 2109 | 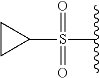 [SMILES: *S(=O)(=O)C1CC1] | 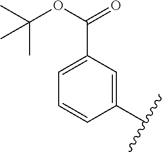 [SMILES: *c1cccc(C(=O)OC(C)(C)C)c1] | N | CH | 1 |
| 2110 | 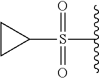 [SMILES: *S(=O)(=O)C1CC1] | 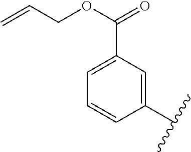 [SMILES: *c1cccc(C(=O)OCC=C)c1] | N | CH | 1 |
| 2111 | H | 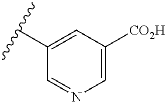 [SMILES: *c1cncc(C(=O)O)c1] | N | CH | 1 |
| 2112 | H | 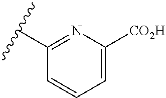 [SMILES: *c1cccc(C(=O)O)n1] | N | CH | 1 |
| 2113 | H | 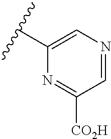 [SMILES: *c1cncc(C(=O)O)n1] | N | CH | 1 |
| 2114 | H | 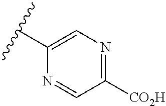 [SMILES: *c1cnc(C(=O)O)cn1] | N | CH | 1 |
| 2115 | H | 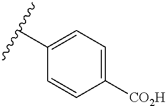 [SMILES: *c1ccc(C(=O)O)cc1] | N | CH | 1 |
| 2116 | H | 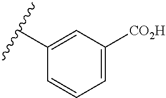 [SMILES: *c1cccc(C(=O)O)c1] | N | CH | 1 |
| 2117 | H | 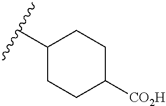 [SMILES: *C1CCC(C(=O)O)CC1] | N | CH | 1 |
| 2118 | H | 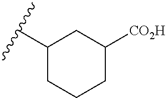 [SMILES: *C1CCCC(C(=O)O)C1] | N | CH | 1 |
| 2119 | H | 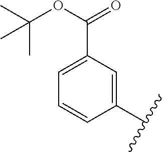 [SMILES: *c1cccc(C(=O)OC(C)(C)C)c1] | N | CH | 1 |
| 2120 | H | 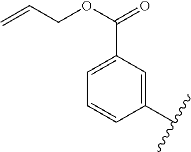 [SMILES: *c1cccc(C(=O)OCC=C)c1] | N | CH | 1 |

| | | | Formula (IIc) |
|---|---|---|---|
| 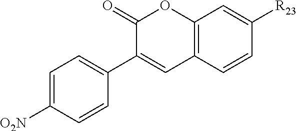 | | | |
| Compound # | R₂₃ | R₂₁ | p |
| 2121 | —OH | N/A | N/A |
| 2122 | 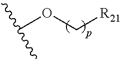 | 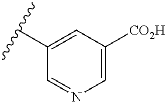 [SMILES: *c1cncc(C(=O)O)c1] | 1 |
| 2123 | 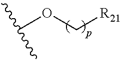 | 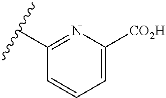 [SMILES: *c1cccc(C(=O)O)n1] | 1 |
| 2124 | 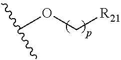 | 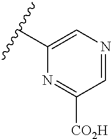 [SMILES: *c1cncc(C(=O)O)n1] | 1 |
| 2125 | 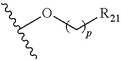 | 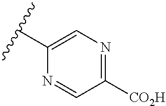 [SMILES: *c1cnc(C(=O)O)cn1] | 1 |
| 2126 | 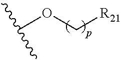 | 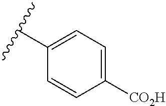 [SMILES: *c1ccc(C(=O)O)cc1] | 1 |
| 2127 | 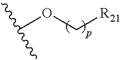 | 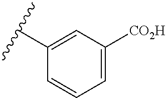 [SMILES: *c1cccc(C(=O)O)c1] | 1 |
| 2128 | 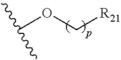 | 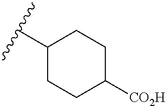 [SMILES: *C1CCC(C(=O)O)CC1] | 1 |
| 2129 | 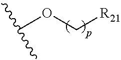 | 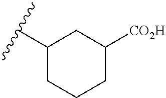 [SMILES: *C1CCCC(C(=O)O)C1] | 1 |
| 2130 | 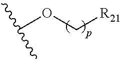 | 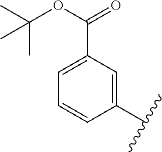 [SMILES: *c1cccc(C(=O)OC(C)(C)C)c1] | 1 |
| 2131 | 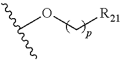 | 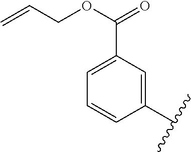 [SMILES: *c1cccc(C(=O)OCC=C)c1] | 1 |

In some embodiments, the compound of Formula (I) is a compound of formula 2046, or a pharmaceutically acceptable salt, solvate, hydrate, cocrystal, or prodrug thereof:

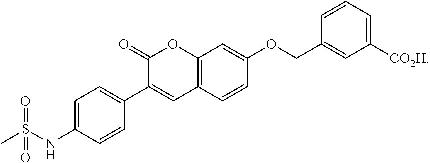 [SMILES: CS(=O)(=O)Nc1ccc(-c2cc3ccc(OCc4cccc(C(=O)O)c4)cc3oc2=O)cc1]

In some embodiments, \textcircled{A} is

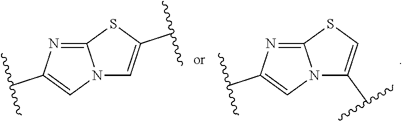 [SMILES: *c1cn2cc(*)sc2n1 ; *c1cn2c(*)csc2n1]

In some embodiments, the compound of Formula (I) is a compound of Formula (IIIa) Formula (IIIb), Formula (IIIc), or a pharmaceutically acceptable salt, solvate, hydrate, cocrystal, or prodrug thereof:

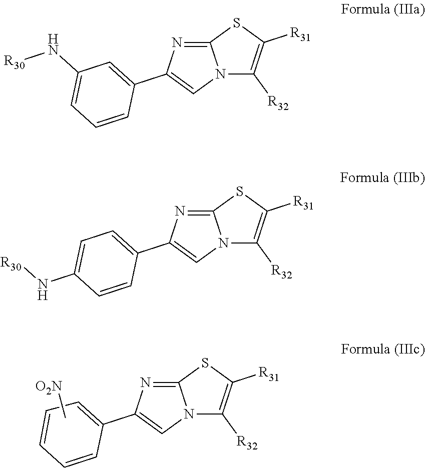

wherein
R₃₀ is selected from H, —S(═O)₂R₃₄, and —C(═O)₂R₃₄;
R₃₁ and R₃₂ are each independently selected from H, alkyl, —OH, —C(═O)H, —C(═O)OH, —C(═O)OR₃₃, and —C(═O)NH₂;
R₃₃ is alkyl or alkenyl; and
R₃₄ is selected from optionally substituted alkyl and optionally substituted cycloalkyl.

In some embodiments, R₃₄ is optionally substituted alkyl. In some embodiments, R₃₄ is optionally substituted C₁-C₆alkyl. In some embodiments, R₃₄ is methyl. In some embodiments, R₃₄ is optionally substituted cycloalkyl. In some embodiments, R₃₄ is optionally substituted C₃-C₆alkyl. In some embodiments, R₃₄ is cyclopropyl. In some embodiments, R₃₄ is selected from methyl and cyclopropyl.

In some embodiments, R₃₀ is selected from H, —S(═O)₂CH₃, —C(═O)CH₃, and

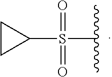 [SMILES: *S(=O)(=O)C1CC1]

In some embodiments, one of R₃ or R₃₂ is —OH, —C(═O)H, —C(═O)OH, —C(═O)OR₃₃, and —C(═O)NH₂.

In some embodiments, the compound is of any one of formulas 3001 to 3073, or a pharmaceutically acceptable salt, solvate, hydrate, cocrystal, or prodrug thereof:

| | |
|---|---|
| | Formula (IIIa) |
| 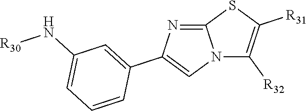 | |

| Com- | | | |
|---|---|---|---|
| pound | | | |
| # | R₃₀ | R₃₁ | R₃₁ |
| 3001 | —S(═O)₂CH₃ | —C(═O)OH | H |
| 3002 | —S(═O)₂CH₃ | —C(═O)OCH₂CH₃ | H |
| 3003 | —S(═O)₂CH₃ | H | —C(═O)OH |
| 3004 | —S(═O)₂CH₃ | H | —C(═O)OCH₂CH₃ |
| 3005 | —S(═O)₂CH₃ | —C(═O)OH | —CH₃ |
| 3006 | —S(═O)₂CH₃ | —C(═O)OCH₂CH₃ | —CH₃ |
| 3007 | —S(═O)₂CH₃ | —CH₃ | —C(═O)OH |
| 3008 | —S(═O)₂CH₃ | —CH₃ | —C(═O)OCH₂CH₃ |
| 3009 | —C(═O)CH₃ | —C(═O)OH | H |
| 3010 | —C(═O)CH₃ | —C(═O)OCH₂CH₃ | H |
| 3011 | —C(═O)CH₃ | H | —C(═O)OH |
| 3012 | —C(═O)CH₃ | H | —C(═O)OCH₂CH₃ |
| 3013 | —C(═O)CH₃ | —C(═O)OH | —CH₃ |
| 3014 | —C(═O)CH₃ | —C(═O)OCH₂CH₃ | —CH₃ |
| 3015 | —C(═O)CH₃ | —CH₃ | —C(═O)OH |
| 3016 | —C(═O)CH₃ | —CH₃ | —C(═O)OCH₂CH₃ |
| 3017 | 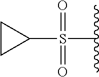 [SMILES: *S(=O)(=O)C1CC1] | —C(═O)OH | H |
| 3018 | 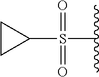 [SMILES: *S(=O)(=O)C1CC1] | —C(═O)OCH₂CH₃ | H |
| 3019 | 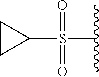 [SMILES: *S(=O)(=O)C1CC1] | H | —C(═O)OH |
| 3020 | 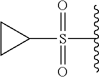 [SMILES: *S(=O)(=O)C1CC1] | H | —C(═O)OCH₂CH₃ |
| 3021 | 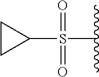 [SMILES: *S(=O)(=O)C1CC1] | —C(═O)OH | —CH₃ |
| 3022 | 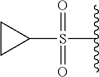 [SMILES: *S(=O)(=O)C1CC1] | —C(═O)OCH₂CH₃ | —CH₃ |
| 3023 | 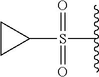 [SMILES: *S(=O)(=O)C1CC1] | —CH₃ | —C(═O)OH |
| 3024 | 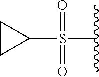 [SMILES: *S(=O)(=O)C1CC1] | —CH₃ | —C(═O)OCH₂CH₃ |
| 3025 | H | —C(═O)OH | H |
| 3026 | H | —C(═O)OCH₂CH₃ | H |
| 3027 | H | H | —C(═O)OH |
| 3028 | H | H | —C(═O)OCH₂CH₃ |
| 3029 | H | —C(═O)OH | —CH₃ |
| 3030 | H | —C(═O)OCH₂CH₃ | —CH₃ |
| 3031 | H | —CH₃ | —C(═O)OH |
| 3032 | H | —CH₃ | —C(═O)OCH₂CH₃ |

| | |
|---|---|
| | Formula (IIIb) |
| 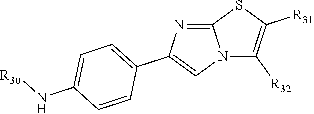 | |

| Com- | | | |
|---|---|---|---|
| pound | | | |
| # | R₃₀ | R₃₁ | R₃₂ |
| 3033 | —S(═O)₂CH₃ | —C(═O)OH | H |
| 3034 | —S(═O)₂CH₃ | —C(═O)OCH₂CH₃ | H |
| 3035 | —S(═O)₂CH₃ | H | —C(═O)OH |
| 3036 | —S(═O)₂CH₃ | H | —C(═O)OCH₂CH₃ |
| 3037 | —S(═O)₂CH₃ | —C(═O)OH | —CH₃ |
| 3038 | —S(═O)₂CH₃ | —C(═O)OCH₂CH₃ | —CH₃ |
| 3039 | —S(═O)₂CH₃ | —CH₃ | —C(═O)OH |
| 3040 | —S(═O)₂CH₃ | —CH₃ | —C(═O)OCH₂CH₃ |
| 3041 | —C(═O)CH₃ | —C(═O)OH | H |
| 3042 | —C(═O)CH₃ | —C(═O)OCH₂CH₃ | H |
| 3043 | —C(═O)CH₃ | H | —C(═O)OH |
| 3044 | —C(═O)CH₃ | H | —C(═O)OCH₂CH₃ |
| 3045 | —C(═O)CH₃ | —C(═O)OH | —CH₃ |
| 3046 | —C(═O)CH₃ | —C(═O)OCH₂CH₃ | —CH₃ |
| 3047 | —C(═O)CH₃ | —CH₃ | —C(═O)OH |
| 3048 | —C(═O)CH₃ | —CH₃ | —C(═O)OCH₂CH₃ |
| 3049 | 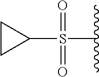 [SMILES: *S(=O)(=O)C1CC1] | —C(═O)OH | H |
| 3050 | 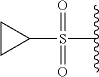 [SMILES: *S(=O)(=O)C1CC1] | —C(═O)OCH₂CH₃ | H |
| 3051 | 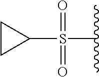 [SMILES: *S(=O)(=O)C1CC1] | H | —C(═O)OH |
| 3052 | 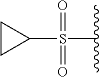 [SMILES: *S(=O)(=O)C1CC1] | H | —C(═O)OCH₂CH₃ |
| 3053 | 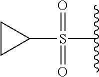 [SMILES: *S(=O)(=O)C1CC1] | —C(═O)OH | —CH₃ |
| 3054 | 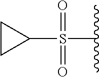 [SMILES: *S(=O)(=O)C1CC1] | —C(═O)OCH₂CH₃ | —CH₃ |
| 3055 | 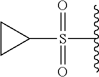 [SMILES: *S(=O)(=O)C1CC1] | —CH₃ | —C(═O)OH |
| 3056 | 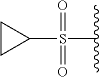 [SMILES: *S(=O)(=O)C1CC1] | —CH₃ | —C(═O)OCH₂CH₃ |
| 3057 | H | —C(═O)OH | H |
| 3058 | H | —C(═O)OCH₂CH₃ | H |
| 3059 | H | H | —C(═O)OH |
| 3060 | H | H | —C(═O)OCH₂CH₃ |
| 3061 | H | —C(═O)OH | —CH₃ |
| 3062 | H | —C(═O)OCH₂CH₃ | —CH₃ |
| 3063 | H | —CH₃ | —C(═O)OH |
| 3064 | H | —CH₃ | —C(═O)OCH₂CH₃ |

| | |
|---|---|
| | Formula (IIIc) |
| 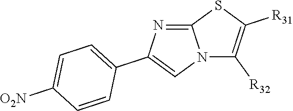 | |

| | R₃₁ | R₃₂ |
|---|---|---|
| 3066 | —C(═O)OH | H |
| 3067 | —C(═O)OCH₂CH₃ | H |
| 3068 | H | —C(═O)OH |
| 3069 | H | —C(═O)OCH₂CH₃ |
| 3070 | —C(═O)OH | —CH₃ |
| 3071 | —C(═O)OCH₂CH₃ | —CH₃ |
| 3072 | —CH₃ | —C(═O)OH |
| 3073 | —CH₃ | —C(═O)OCH₂CH₃ |

In embodiments, the compound of Formula (I) is a compound of formula 3035, or a pharmaceutically acceptable salt, solvate, hydrate, cocrystal, or prodrug thereof:

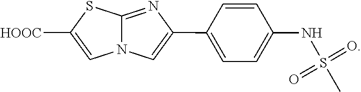

In embodiments, the compound of Formula (I) is a compound of formula 3037, or a pharmaceutically acceptable salt, solvate, hydrate, cocrystal, or prodrug thereof:

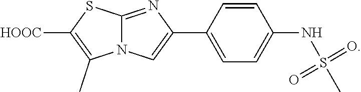

### Methods of Treatment

The compounds and compositions described herein can be used in methods for treating diseases and conditions. In some embodiments, the compounds and compositions described herein, for example, and without limitation, compounds of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073 or compositions thereof, can be used in the methods for treating a disease or a condition associated with the inhibition of alcohol dehydrogenase-2 (ALDH2) protein. In some embodiments, the compounds and compositions described herein for example, and without limitation, compounds of Formula (I), Formula (IIa), Formula (IIb), Formula (IIc), Formula (20), Formula (21), Formula (IIIa), Formula (IIIb), Formula (IIc), Formulas 1001-1008, Formulas 2001-2131, or Formulas 3001-3073 or compositions thereof, can be used to inhibit liver-specific alcohol dehydrogenase-2 (ALDH2) protein. In some embodiments, inhibition is in vitro, while in other embodiments, inhibition is in vivo. In some embodiments, the compounds and compositions described herein for example, and without limitation, compounds of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073 or compositions thereof, can be actively taken up as a substrate for a liver-specific organic anion transporting polypeptide (OATP) transporter.

### Pharmaceutical Compositions

In an embodiment, the disclosure provides a pharmaceutical composition for use in the treatment of the diseases and conditions described herein.

The pharmaceutical compositions are typically formulated to provide a therapeutically effective amount of a compound of Formula (I), Formula (IIa), Formula (IIb), Formula (IIc), Formula (20), Formula (21), Formula (IIIa), Formula (IIb), Formula (IIIc), Formulas 1001-1008, Formulas 2001-2131, or Formulas 3001-3073, and their features and limitations as described herein, or a pharmaceutically acceptable salt, solvate, or hydrate thereof, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of sepsis, meningitis, endocarditis, osteomyelitis, otitis media, sinusitis, pneumonia, chronic respiratory tract infection, catheter infection, postoperative peritonitis, postoperative biliary tract, tricuspid valve endocarditis, ecthyma gangrenosum, eyelid abscess, lacrimal cystitis, conjunctivitis, corneal ulcer, corneal abscess, panophthalmitis, orbital infection, urinary tract infection, complicated urinary tract infection, catheter infection, perianal abscess, severe burns, airway burns, pressure ulcer infections, cystic fibrosis, a bacterial infection, and cancer.

In some embodiments, the concentration of a compound of Formula (I), Formula (IIa), Formula (IIb), Formula (IIc), Formula (20), Formula (21), Formula (IIIa), Formula (IIIb), Formula (IIIc), Formula 1001-1008, Formulas 2001-2131, or Formula 3001-3073, and their features and limitations as described herein, or a pharmaceutically acceptable salt thereof, provided in the pharmaceutical compositions of the disclosure is less than, for example, 100%, 90%, 80%7, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3% 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, provided in the pharmaceutical compositions of the disclosure is independently greater than 90%, 80%, 70%, 60%0, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75% 4.50%, 4.25%, 4%, 3.75% 3.50%, 3.25%, 3%, 2.75%, 2.50% 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound of Formula (I), Formula (IIa), Formula (IIb), Formula (IIc), Formula (20), Formula (21), Formula (IIIa), Formula (IIb), Formula (IIc), Formulas 1001-1008, Formulas 2001-2131, or Formulas 3001-3073, and their features and limitations as described herein, or pharmaceutically acceptable salt thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound of Formula (I), Formula (IIa), Formula (IIb), Formula (IIc), Formula (20), Formula (21), Formula (IIIa), Formula (IIb), Formula (IIIc), Formulas 1001-1008, Formulas 2001-2131, or Formulas 3001-3073, and their features and limitations as described herein, or pharmaceutically acceptable salt thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of Formula (I), Formula (IIa), Formula (IIb), Formula (IIc), Formula (20), Formula (21), Formula (IIIa), Formula (IIb), Formula (IIc), Formulas 1001-1008, Formulas 2001-2131, or Formulas 3001-3073, and their features and limitations as described herein, or pharmaceutically acceptable salt thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing: a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, and a pharmaceutical excipient suitable for administration.

In preferred embodiments, the disclosure provides a solid pharmaceutical composition for oral administration containing: (i) an effective amount of: a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, and (ii) a pharmaceutical excipient suitable for administration. In some embodiments, the composition further contains (iii) an effective amount of an additional active pharmaceutical ingredient. For example, additional active pharmaceutical ingredients, as used herein, may include one or more compounds that induce cell cycle arrest and/or apoptosis in cells containing functional Mcl-1 and/or Bcl-2 proteins. Such additional active pharmaceutical ingredients may also include those compounds used for sensitizing cells to additional agent(s), such as inducers of apoptosis and/or cell cycle arrest, and chemoprotection of normal cells through the induction of cell cycle arrest prior to treatment with chemotherapeutic agents.

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 5-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating: a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing: a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of: a compound of Formula (I), Formula (IIa), Formula (IIb), Formula (IIc), Formula (20), Formula (21), Formula (IIIa), Formula (IIb), Formula (IIIc), Formulas 1001-1008, Formulas 2001-2131, or Formulas 3001-3073, and their features and limitations as described herein, or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of: a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, can also be administered intraadiposally or intrathecally.

The compositions of the disclosure may also be delivered via an impregnated or coated device such as a stent, for example, or an artery-inserted cylindrical polymer. In some embodiments, the compounds and compositions of the disclosure can be used in conjunction with stents to treat or prevent infections and/or biofilms in the blood vessels or heart. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. A compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, may additionally be administered intravascularly from a balloon used during angioplasty. Extravascular administration of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, via the pericard or via advential application of formulations of the disclosure may also be performed.

Exemplary parenteral administration forms include solutions or suspensions of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, in sterile aqueous solutions, for example, aqueous propylene glycol or dextrose solutions. Such dosage forms can be suitably buffered, if desired.

The disclosure also provides kits. The kits include a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the compound of Formula (I), Formula (IIa), Formula (IIb), Formula (IIc), Formula (20), Formula (21), Formula (IIIa), Formula (IIb), Formula (IIIc), Formulas 1001-1008, Formulas 2001-2131, or Formulas 3001-3073, and their features and limitations as described herein, or pharmaceutically acceptable salt thereof, described herein, and another active pharmaceutical ingredient are provided as separate compositions in separate containers within the kit. In some embodiments, the compound of Formula (I), Formula (IIa), Formula (IIb), Formula (IIc), Formula (20), Formula (21), Formula (IIIa), Formula (IIIb), Formula (IIc), Formulas 1001-1008, Formulas 2001-2131, or Formulas 3001-3073, and their features and limitations as described herein, or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of conditions associated with inhibition of alcohol dehydrogenase-2 (ALDH2) protein.

In some embodiments, the kits described herein are for use in the treatment of an alcohol use disorder.

### Dosages and Dosing Regimens

The amounts of: a compound of Formula (I), Formula (IIa), Formula (IIb), Formula (IIc), Formula (20), Formula (21), Formula (IIIa), Formula (IIb), Formula (IIIc), Formulas 1001-1008, Formulas 2001-2131, or Formulas 3001-3073, and their features and limitations as described herein, or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, may be provided in units of mg/kg of body mass or in mg/m² of body surface area.

In some embodiments, a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein is administered in multiple doses. In a preferred embodiment, a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, is administered about once per day to about 6 times per day. In some embodiments a compound of Formula (I), Formula (IIa), Formula (IIb), Formula (IIc), Formula (20), Formula (21), Formula (IIa), Formula (IIb), Formula (IIc), Formulas 1001-1008, Formulas 2001-2131, or Formulas 3001-3073, and their features and limitations as described herein, or pharmaceutically acceptable salt thereof, described herein, is administered once daily, while in other embodiments, a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein is administered twice daily, and in other embodiments a compound Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, is administered three times daily.

Administration of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, may continue as long as necessary. In some embodiments, a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, is administered for more than 1, 2, 3, 4, 5, 6, 7, 14, or 28 days. In some embodiments a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein is administered for less than 28, 14, 7, 6, 5, 4, 3, 2, or 1 day. In some embodiments, a compound Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein is administered chronically on an ongoing basis—e.g., for the treatment of chronic effects. In another embodiment, the administration of a compound of Formula (I), Formula (IIa), Formula (IIb), Formula (IIc), Formula (20), Formula (21), Formula (IIIa), Formula (IIb), Formula (IIIc), Formulas 1001-1008, Formulas 2001-2131, or Formulas 3001-3073, and their features and limitations as described herein, or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a compound of Formula (I), Formula (IIa), Formula (IIb), Formula (IIc), Formula (20), Formula (21), Formula (IIIa), Formula (IIIb), Formula (IIIc), Formulas 1001-1008, Formulas 2001-2131, or Formulas 3001-3073, and their features and limitations as described herein, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### Clauses of the Disclosure

The following clauses describe certain embodiments.

Clause 1. A compound of Formula (I), or a pharmaceutically acceptable salt, solvate, hydrate, cocrystal, or prodrug thereof.

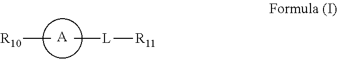

wherein
\textcircled{A} is optionally substituted heteroaryl;
R₁₀ is selected from halo, optionally substituted aryl, optionally substituted heteroaryl;
R₁₁ is selected from —OH, —C(═O)H, —C(═O)OH, —C(═O)OR₁₃, and —C(═O)NH₂;
L is a linker;
R₁₃ is selected from optionally substituted alkyl and optionally substituted alkenyl.

Clause 2. The compound of clause 1, wherein L is a bond or —O—(CH₂)ₙ—R₁₄—;
wherein R₁₄ is selected from optionally substituted cycloalkyl, optionally substituted heterocyclyl, optionally substituted aryl, and optionally substituted heteroaryl; and
n is an integer selected from 1, 2, 3, 4, 5, or 6.

Clause 3. The compound of clause 1 or 2, wherein
R₁₀ is F, Br, Cl, I,

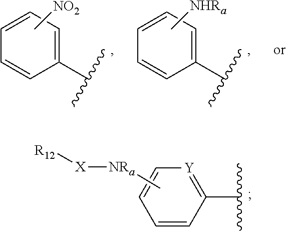

X is selected from —C(═O)— and —S(═O)₂—
Y is selected from CH and N;
R₁₂ is selected from optionally substituted alkyl, optionally substituted cycloalkyl, optionally substituted heterocyclyl, optionally substituted aryl, and optionally substituted heteroaryl; and
R^{a} is selected from H and optionally substituted alkyl.

Clause 4. The compound of any one of Clauses 1-3, wherein:
\textcircled{A} is

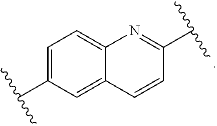 [SMILES: *c1ccc2nc(*)ccc2c1]

Clause 5. The compound of Clause 4, wherein
L is —O—(CH₂)ₙ—R₁₄—;
R₁₀ is Cl or

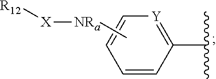

R₁₁ is selected from —C(═O)OH and —C(═O)OR₁₃;
R₁₂ is alkyl; and
R₁₄ is C₆ aryl.

Clause 6. The compound of Clause 4 or Clause 5, wherein the compound is selected from Formula 1001 to Formula 1008, or a pharmaceutically acceptable salt, solvate, hydrate, cocrystal, or prodrug thereof.

Clause 7. The compound of Clause 4, wherein the compound of Formula (I) is a compound of formula 1007:

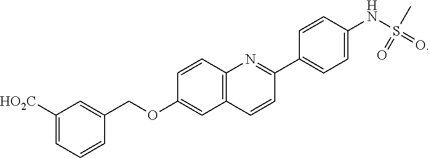 [SMILES: CS(=O)(=O)Nc1ccc(-c2ccc3cc(OCc4cccc(C(=O)O)c4)ccc3n2)cc1]

Clause 8. The compound of Clause 1, wherein:
\textcircled{A} is

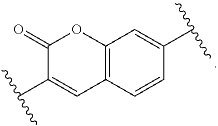 [SMILES: *c1ccc2cc(*)c(=O)oc2c1]

Clause 9. The compound of Clause 1 or Clause 8, wherein the compound of Formula (I) is a compound of Formula (IIa) Formula (IIb), Formula (IIc), or a pharmaceutically acceptable salt, solvate, hydrate, cocrystal, or prodrug thereof:

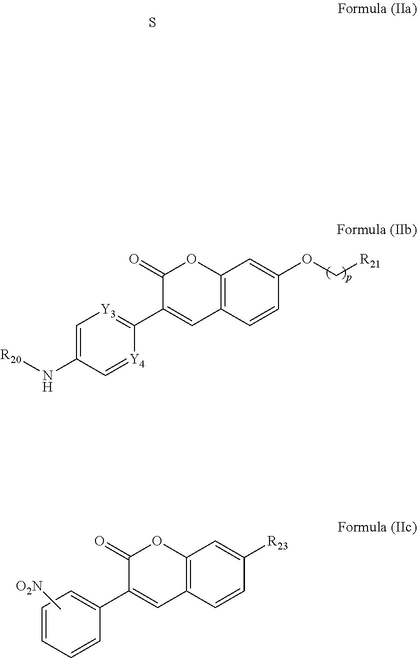

wherein
Y₁ and Y₂ are each CH, Y₁ is CH and Y₂ is N, or Y₁ is N and Y₂ is CH;
Y₃ and Y₄ are each CH, Y₃ is CH and Y₄ is N, or Y₃ is N and Y₄ is CH;
R₂₀ is selected from H, —S(═O)₂R₂₄, and —C(═O)₂R₂₄;
R₂₁ is selected from —C(O)OH, optionally substituted aryl, optionally substituted heteroaryl, and optionally substituted cycloalkyl;
R₂₃ is selected from —OH and

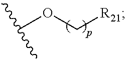

and
R₂₄ is selected from optionally substituted alkyl and optionally substituted cycloalkyl; and
p is an integer selected from 1-14.

Clause 9. The compound of Clause 8, wherein R₂₄ is selected from methyl and cyclopropyl.

Clause 10. The compound of Clause 8 or Clause 9, wherein R₂₀ is selected from H, —S(═O)₂CH₃, —C(═O)CH₃, and

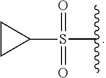 [SMILES: *S(=O)(=O)C1CC1]

Clause 12. The compound of any one of Clauses 9-11, wherein the compound of Formula (IIb) is a compound of Formula (20), or a pharmaceutically acceptable salt, solvate, hydrate, cocrystal, or prodrug thereof:

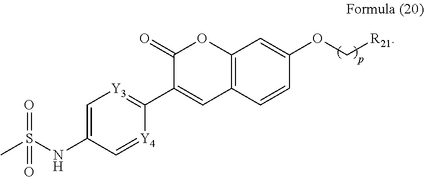

Clause 13. The compound of any one of Clauses 9-12, wherein R₂₁ is selected from optionally substituted phenyl, optionally substituted cyclohexyl, optionally substituted pyridine, and optionally substituted pyrazine.

Clause 14. The compound of any one of Clauses 9-13, wherein R₂₁ is selected from

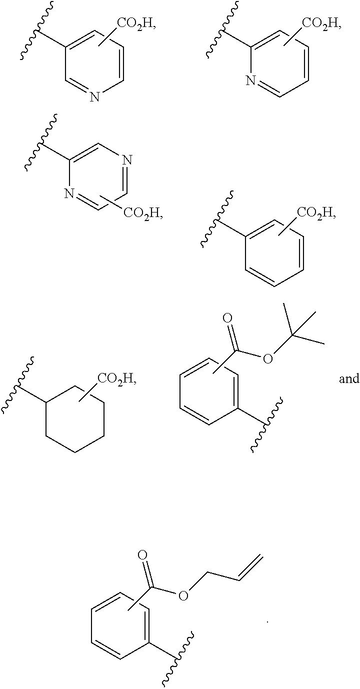

Clause 15. The compound of any one of Clauses 9-14, wherein p is 1.

Clause 16. The compound of any one of Clauses 9-15, wherein Y₁ and Y₂ are each CH.

Clause 17. The compound of any one of Clauses 9-15, wherein Y₃ and Y₄ are each CH.

Clause 18. The compound of any one of Clauses 9-15, wherein Y₃ is N and Y₄ is CH.

Clause 19. The compound of any one of Clauses 9-12, wherein the compound of Formula (IIb) is a compound of Formula (21), or a pharmaceutically acceptable salt, solvate, hydrate, cocrystal, or prodrug thereof:

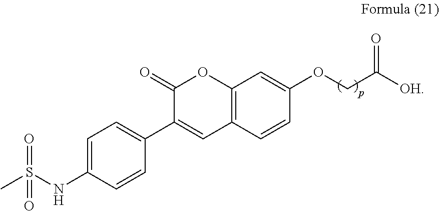

Clause 20. The compound of Clause 19, wherein p is an integer from 4-14.

Clause 21. The compound of Clause 9, wherein the compound is of any one of formulas 2001 to 2131, or a pharmaceutically acceptable salt, solvate, hydrate, cocrystal, or prodrug thereof.

Clause 22. The compound of Clause 21, wherein the compound of Formula (I) is a compound of formula 2046, or a pharmaceutically acceptable salt, solvate, hydrate, cocrystal, or prodrug thereof:

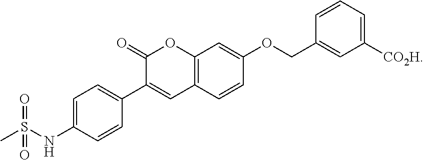 [SMILES: CS(=O)(=O)Nc1ccc(-c2cc3ccc(OCc4cccc(C(=O)O)c4)cc3oc2=O)cc1]

Clause 23. The compound of Clause 1, wherein:
\textcircled{A} is

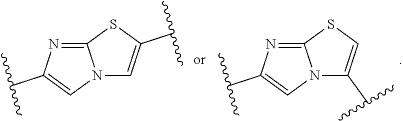 [SMILES: *c1cn2cc(*)sc2n1 ; *c1cn2c(*)csc2n1]

Clause 24. The compound of Clause 23, wherein the compound of Formula (I) is a compound of Formula (IIIa) Formula (IIIb), Formula (IIIc), or a pharmaceutically acceptable salt, solvate, hydrate, cocrystal, or prodrug thereof:

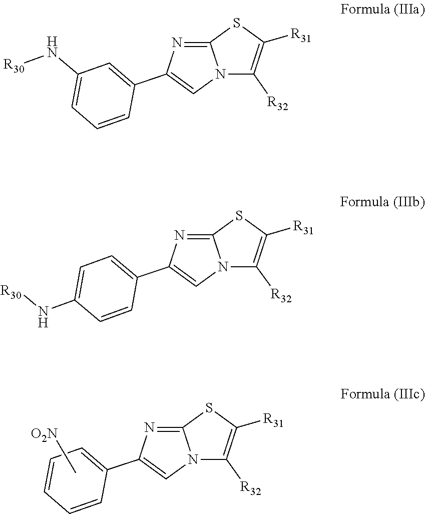

wherein
R₃₀ is selected from H, —S(═O)₂R₃₄, and —C(═O)₂R₃₄;
R₃₁ and R₃₂ are each independently selected from H, alkyl, —OH, —C(═O)H, —C(═O)OH, —C(═O)OR₃₃, and —C(═O)NH₂;
R₃₃ is alkyl or alkenyl; and
R₃₄ is selected from optionally substituted alkyl and optionally substituted cycloalkyl

Clause 25. The compound of Clause 23 or Clause 24, wherein R₃₀ is selected from H, —S(═O)₂CH₃, —C(═O)CH₃, and

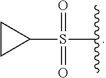 [SMILES: *S(=O)(=O)C1CC1]

Clause 26. The compound of Clause 24 or Clause 25, wherein one of R₃₁ or R₃₂ is —OH, —C(═O)H, —C(═O)OH, —C(═O)OR₃₃, and —C(═O)NH₂.

Clause 27. The compound of Clause 24, wherein the compound is of any one of formulas 3001 to 3073, or a pharmaceutically acceptable salt, solvate, hydrate, cocrystal, or prodrug thereof.

Clause 28. The compound of Clause 27, wherein the compound of Formula (I) is a compound of formula 3035, or a pharmaceutically acceptable salt, solvate, hydrate, cocrystal, or prodrug thereof:

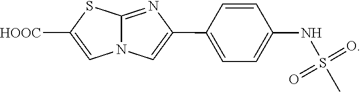

Clause 29. The compound of Clause 27, wherein the compound of Formula (I) is a compound of formula 3037, or a pharmaceutically acceptable salt, solvate, hydrate, cocrystal, or prodrug thereof:

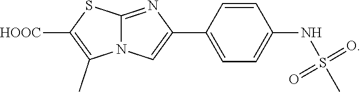

Clause 30. The compound of any one of Clauses 1-29, wherein the compound inhibits alcohol dehydrogenase-2 (ALDH2) protein.

Clause 31. The compound of any one of Clauses 1-30, wherein the compound inhibits liver-specific alcohol dehydrogenase-2 (ALDH2) protein.

Clause 32. The compound of any one of Clauses 1-31, wherein the compound can be actively taken up as a substrate for a liver-specific organic anion transporting polypeptide (OATP) transporter.

Clause 33. A pharmaceutical composition comprising a compound of any one of Clauses 1-32 or a pharmaceutically acceptable salt, solvate, hydrate, cocrystal, or prodrug thereof, and a pharmaceutically acceptable carrier or excipient.

Clause 34. A method of treating a condition by inhibiting alcohol dehydrogenase-2 (ALDH2) protein activity in a patient in need of said treatment, the method comprising administering to the patient a therapeutically effective amount of a compound of any of Clauses 1 to 32, or a pharmaceutically acceptable salt, solvate, hydrate, cocrystal, or prodrug thereof.

Clause 35. A method of treating a condition by inhibiting alcohol dehydrogenase-2 (ALDH2) protein activity in a patient in need of said treatment, the method comprising administering to the patient a therapeutically effective amount of a therapeutically effective amount of the pharmaceutical composition of Clause 33.

Clause 36. The method of Clause 34 or Clause 35, wherein the compound inhibits liver-specific ALDH2 protein.

Clause 37. The method of Clause 34 or Clause 35, wherein the condition is an alcohol use disorder.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Liver Specific Targeting of ALDH2 for the Treatment of Alcohol Use Disorders

Example 1 describes the synthesis of a series of YA7068 analogs and testing thereof using established assays in vitro and in vivo, to validate and characterize potent and liver-specific ALDH2 inhibitors in treating AUDs. To develop liver-specific ALDH2 inhibitors, a novel coumarin-based small molecule YA7068 was synthesized using computer-aided virtual screening in combination with structure-based drug design (SBDD). Studies have shown YA7068 inhibits the enzymatic activity of ALDH2 with an excellent potency (IC₅₀=62 nM). Simultaneously, YA7068 functions as a substrate for the hepatocyte-specific organic anion transporting polypeptide 1B1 (OATP1B1), leading to liver-selective targeting of ALDH2. In addition, using the available ALDH2 crystal structures, the surface of the enzyme was characterized using a novel computer-aided drug design (CADD) method SILCS (site identification of ligand competitive saturation). The SILCS analyses revealed the presence of a “sweet spot” that maps to the substrate entrance pocket near the ALDH2 active site. While not wishing to be bound by any particular theory, these results suggest further optimization, through coupling compound activity at both sites, combined with an in-depth structure-activity relationship (SAR), leading to the discovery of potent inhibitors for ALDH2, while retaining their OATP1B1 substrate activity. As described herein, computer-aided drug design (CADD) is combined with medicinal chemistry, along with validated in vitro and in vivo assays to identify and characterize compounds that can be developed into potent and liver-specific inhibitors for ALDH2.

Liver-specific inhibition of acetaldehyde metabolism by targeting the enzymatic activity of ALDH2 is a novel therapeutic strategy for the treatment of AUDs. A series of YA7068 analogs are synthesized and tested using established assays in vitro and in vivo in order to validate and characterize potent and liver-specific ALDH2 inhibitors in treating AUDs. In a non-limiting embodiment, small molecule ALDH2 inhibitors are synthesized and tested as follows: 1) a new class of ALDH2 inhibitors designed by the CADD method SILCS based on YA7068 are synthesized; 2) inhibitory potency of new compounds is assayed with a high throughput ALDH2 enzymatic assay; 3) the selectivity of new compounds is tested against other isozymes of the ALDH family; 4) new compounds are studied for their liver-specific uptake by the OATP1 transporters using OATP1B1- and OATP1B3-expressing HEK293 cells an established LC-MS assays; 5) evaluating cellular toxicity of selected compounds in primary human hepatocytes. In a non-limiting example, the pharmacological consequences of liver-specific inhibition of ALDH2 are tested by: 1) determining pharmacokinetic characters (clearance rate, volume of distribution, Cₘₐₓ, and in vivo half-life) of selected compounds; 2) establishing the maximum tolerated doses of selected compounds in vivo; 3) assessing the in vivo efficacy of selected compounds in mouse AUD models. This strategy yields the necessary evidence to assess the therapeutic potential of liver specific inhibition of ALDH2 as a treatment for AUDs.

An exemplary innovation is the development of a first-in-class small molecule that not only inhibits the enzymatic activity of ALDH2 with high potency and isozyme selectivity, but also can be actively taken up as a substrate for the liver-specific OATP1 transporters (e.g., OATP1B1 and OATPIB3). This liver-targeted strategy reduces the potential side effects associated with systematic inhibition of ALDH2.

From the biochemical perspective, the active site of ALDH2 has been exploited to facilitate the rational design of new inhibitors. Site characterization and inhibitor design are driven by a novel target-based CADD method SILCS, which is used together with medicinal chemistry, structural studies, and biological assays in an iterative approach to improve the pharmacodynamic (PD) and pharmacokinetic (PK) properties of inhibitors.

### Experimental Results

Computer-based virtual screening was combined with SBDD to identify chemotypes that were further improved into novel ALDH2 inhibitors. The inhibitory potency of new compounds was determined. The liver uptake of new compounds has been tested using a cellular assay employing the HEK293-OATP1B1 cells.

### Computational Characterization of ALDH2.

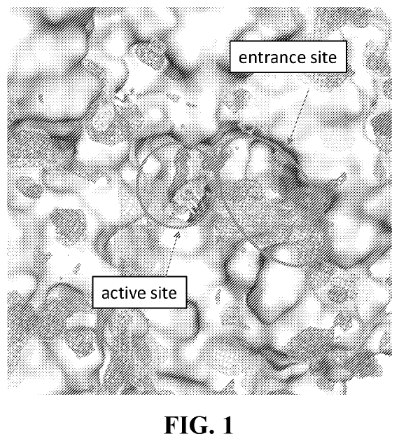

CADD method SILCS, effectively employed to design novel ligands in efforts targeting other protein targets such as transcriptional factor BCL6,46 GPCR mGluR5, and heme oxygenase HemO, was applied to map the functional group requirement to direct new ligand design (FIG. 1). Using SILCS, the 3D functionality probability distribution maps (termed FragMaps) were obtained that identify surface regions where different functional groups have favorable interactions. Specifically, SILCS allows for 1) qualitative analysis of the binding pocket to drive the design of modifications and 2) quantitative predictions of changes in binding affinity for designed modifications. FIG. 1 shows the targeted active site and substrate entrance site. CADD tools were previously used to design novel ligands in efforts targeting other protein targets including transcriptional factor BCL6, GPCR mGluR5, and heme oxygenase HemO.

### Identification of ALDH2 Inhibitors by Virtual Screening.

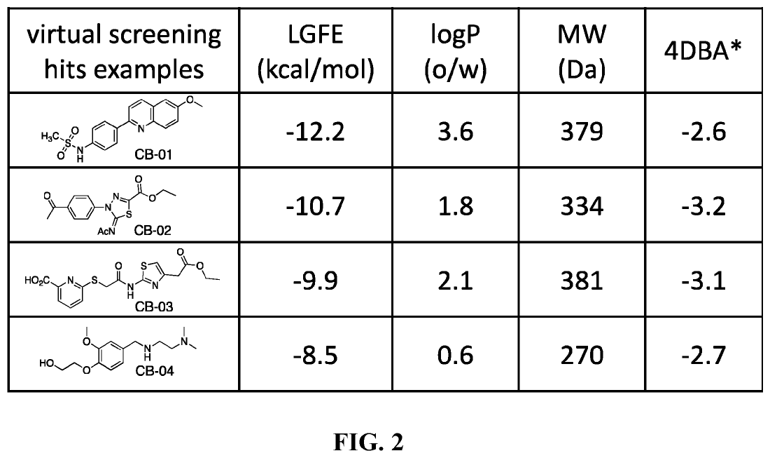

All known ALDH2 inhibitors bind to the active site of the enzyme. Therefore, this region was selected for CADD studies to identify low-molecular-weight competitive inhibitors of ALDH2. The CADD SILCS FragMaps were used to direct a virtual screen of a database of >800,000 commercially available compounds (ChemBridge collection). Screening involved two rounds of docking of the compounds into the putative binding site with final compound selection based on maximizing both chemical diversity and physical properties associated with druglike characteristics. In the first round of the screen, 50,000 compounds having the most favorable N-normalized van der Waals (vdW) attractive energy were selected. This normalization procedure centers the median molecular weight distribution of selected compounds to ˜300 Da, consistent with the known molecular weight (MW) distribution of drug-like molecules. In addition, the use of the vdW attractive energy for ranking eliminates compounds that do not sterically complement the putative binding pocket. In the second round of docking, additional conformations of ALDH2 were included to partially account for the conformational flexibility of the binding pocket. These additional conformations were obtained from a molecular dynamics (MD) simulation of the apo (unliganded) form of ALDH2. This more rigorous second-step screening yielded a final ranking of 1000 compounds as evaluated with the N2/5-normalized total interaction energy (selected from the initial 50,000 compounds). The 1,000 compounds were then subjected to chemical diversity clustering that yielded ˜100 groups, consisting of compounds with related chemical structures. One or two compounds were then selected from each group based on maximizing adherence to Lipinski's Rule of Five, yielding a total of 199 compounds. The CADD-identified inhibitors were predicted to bind in the vicinity of the ALDH2 active site, assuming a variety of orientations within the enzyme active site. A total of 100 of the final 199 hits were available from commercial vendors for experimental testing. As examples, in FIG. 2, the chemical structures of four virtual screening hits, CB-01 to CB-04, were listed along with the ligand grid free energy (LGFE) values, partition coefficient log P, MW, and the druglike parameter 4DBA. The predicted binding mode of hit CB-01 to the active site of ALDH2 has been shown in FIG. 1. The sulfonamide substituted phenyl group fit snugly into the bottom of the substrate-binding channel and form H-Bonds with catalytic residues. The quinoline fragment occupies the substrate channel by forming stacking interactions with the aromatic sidechains of residues F170, F296, F459, and W177. The OMe group points to the outside of the protein surface and is ready for expansion of the structure.

### YA7068 Developed Based on CB-01 as a Potent ALDH2 Inhibitor.

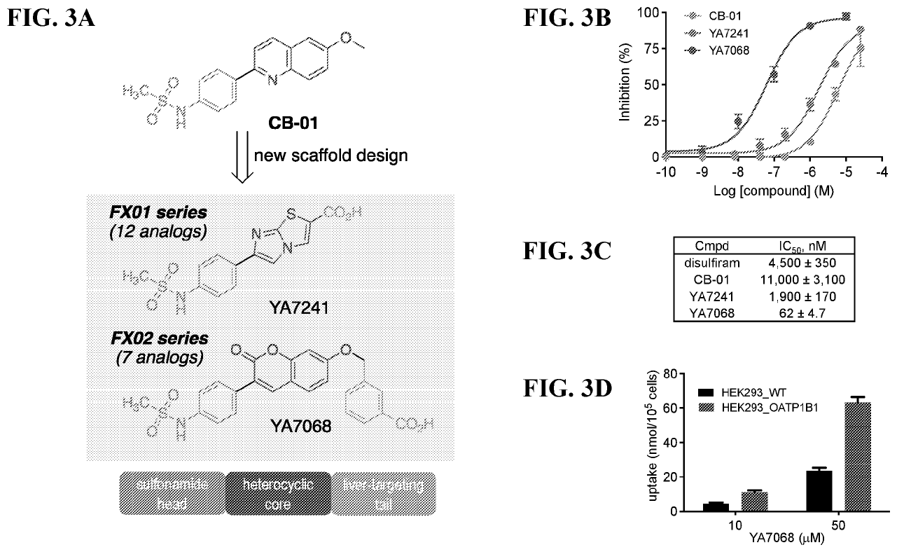

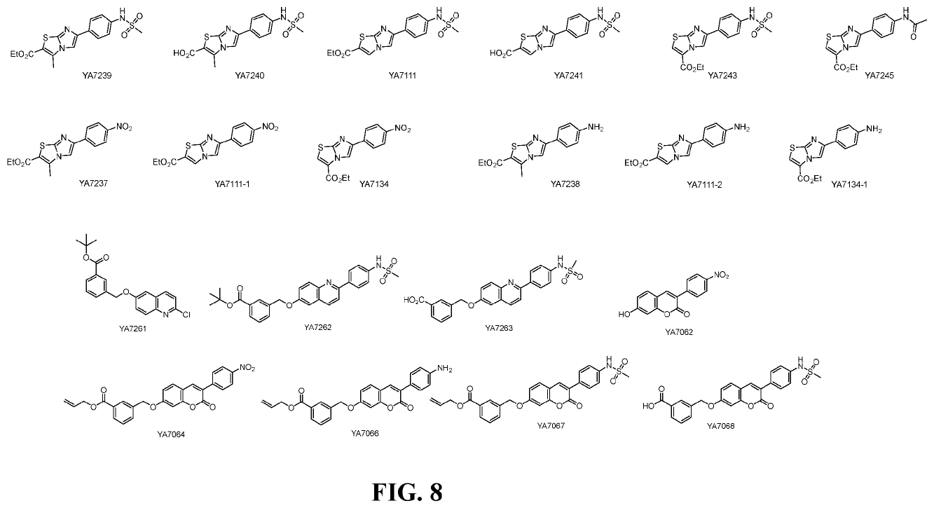

Nineteen new compounds were synthesized that belong to two novel chemotypes FX01-FX02, designed based on the chemical structure of the screening hit CB-01 (FIGS. 3A and 8). The structures of chemotypes FX01-FX02 include three fragments 1) a phenyl-sulfonamide group inherited from the hit CB-01, 2) an aromatic ring system (e.g., a coumarin ring in YA7068 and an imidazothiazole in YA7241) designed to occupy the substrate-binding channel of the enzyme, and 3) a carboxylic acid moiety that extrudes out of the protein surface and serves as recognition functionality for liver-specific uptake by OATP1B1/1B3 transporters (FIG. 3A). Fluorescence-based ALDH2 assay was used to test the potencies of new compounds. While YA7241 indicated an IC₅₀ value of 1.9 μM, similar to that of disulfiram and 5.8-fold more potent than hit CB-01, the coumarin compound YA7068 indicated an IC₅₀ value of 62 nM (FIG. 3B, 3C), which is >170-fold more potent than its parent CB-01 (FIG. 3C).

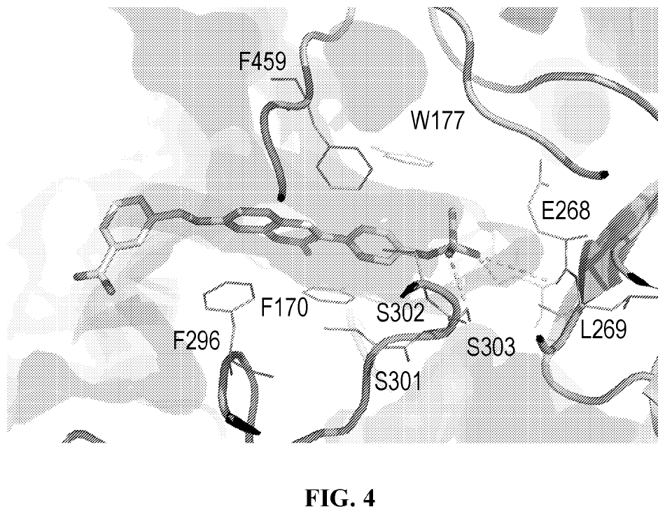

To further characterize the binding interaction between YA7068 and the enzyme, a CADD docking study of the YA7068-ALDH2 complex (FIG. 4) was performed. In the substrate-binding site of ALDH2, the ligand YA7068 adopts an extended conformation to occupy the deep substrate binding channel of ALDH2, with the sulfonamide group of YA7068 pointed to the bottom polar pocket of the active site (FIG. 4), forming H-bonds with the side chains of residues S303 and T244, as well as the backbone NH group of residue L269. The phenyl substituted coumarin system fits snuggly into the substrate channel formed by aromatic side chains of residues F170, W177, F459, and F296. Meanwhile, the OATP1 transporter-targeting benzoate fragment of YA7068 extruded out of the active side and occupied the entrance site of ALDH2.

### YA7068 Identified as a Substrate of OATP1B1.

The uptake of inhibitor YA7068 by the liver-specific transporter OATP1B1 has been evaluated using a protocol similar to the one in a recent study on OCT transporters by Obianom et al. After incubating OATP1B1-transfected HEK293 cells with the compound, the intracellular concentrations of YA7068 was >2.5-fold higher than that after incubating the wild-type HEK293 cells with the compound (FIG. 3D). In sum, compound YA7068 is developed as a novel liver-targeting potent inhibitor of ALDH2.

### Analog Development

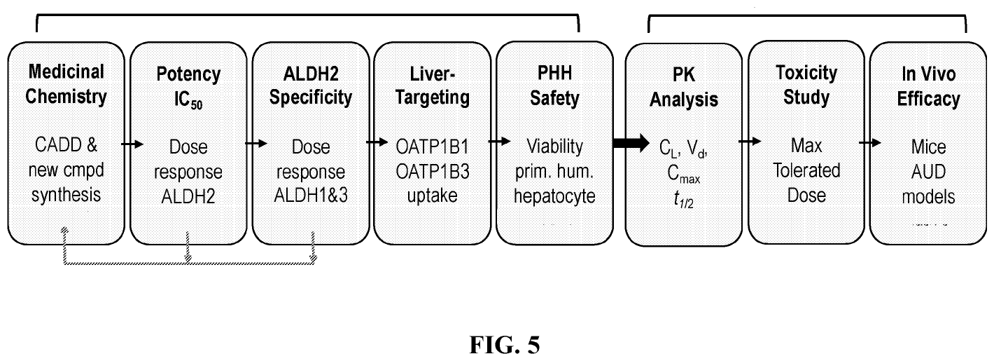

The development of YA7068 analogs targeting the enzymatic activity of ALDH2 in combination with liver-specific OATP1-dependent uptake provides a multi-target strategy, increasing efficacy while decreasing the propensity for the potential toxicity of global inhibition of ALDH2. Expertise in CADD, chemistry, and validated assays were utilized to develop YA7068 analogs with dual activities (FIG. 5). 36 new YA7068 analogs designed by CADD are synthesized. After determining the inhibitory potency, those with IC₅₀<1 μM are tested further forg the inhibition of closely related isozymes such as ALDH1 and ALDH3 using similar enzymatic assays to that of ALDH2. The liver-specific uptake of YA7068 and synthesized new analogs by the OATP1 transporters (eg, OATP1B1 and OATP1B3) are quantified by comparing intracellular inhibitor levels with or without the expression of the OATP1 transporters on the cell surface. The intracellular concentration of testing compounds is followed by LC-MS. The cellular toxicity of compounds is evaluated using primary human hepatocytes. These efforts yield one top compound prioritized for further evaluations. The PK and toxicity of the top compound is determined. For this compound, in vivo efficacy is tested in AUD mouse models. These studies provide a tractable lead that simultaneously inhibits ALDH2 while actively being taken up as a substrate of the liver-specific OATP1 transporters such as OATP1B1 and OATP1B3.

### Small Molecule ALDH2 Inhibitors Synthesis and Testing

New analogs are synthesized and the workflow for testing the potency, selectivity, and cellular toxicity of the new compounds employing SAR, ALDH isozyme assays, liver-uptake studies, and primary human hepatocyte viability assays is detailed herein.

### Synthesizing New ALDH2 Inhibitors Based on YA7068.

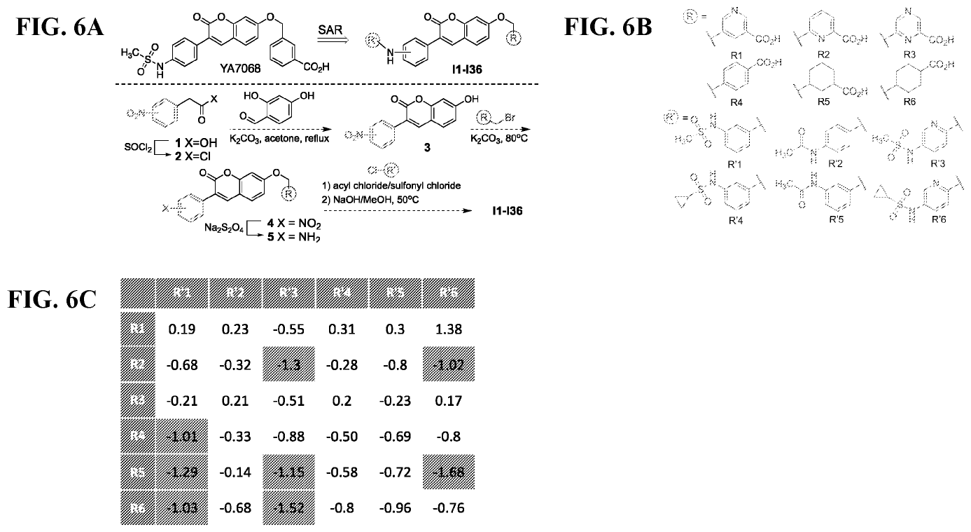

YA7068 was shown to bind to ALDH2 and inhibit its enzymatic activity while simultaneously acting as a substrate for OATP1B1 (FIGS. 3A-3D). The CADD methodology SILCS was used to design compounds that bind to the active site and the substrate entrance site of ALDH2 (FIG. 1). The benzoic acid fragment of compound YA7068 can occupy the substrate entrance pocket (FIG. 4). To further enhance the potency and selectivity of inhibitors, specific modifications are evaluated for their biological activities. The synthesis of I1-I36 begins with 1 (FIG. 6A). Activation of acid 1 with SOCl₂ gives acyl chloride 2, which is submitted to a ring-closing cyclization reaction in the presence of K₂CO₃ to generate coumarin 3. Alkylation of the phenol group in compound 3 with various bromides (FIG. 6B) generates nitro 4, which is then reduced using sodium hydrosulfite to give amino compound 5. Finally, the amino group of compound 5 is allowed to react with various acyl chlorides or sulfonyl chlorides (FIG. 6B) to yield the final inhibitors I1-I36. Presented in FIG. 6C are the SILCS LGFE differences (kcal/mol) for the new compounds vs YA7068 (LGFE −8.20 kcal/mol). Red are modifications that lead to the most favorable LGFE values while yellow indicates those within 0.5-1.0 kcal/mol to YA7068. Priority is given to the ones shown in red followed by those in yellow.

### Determine Inhibitory Potency Using Enzymatic Activity Assay.

IC₅₀ values of synthesized compounds is tested by an ALDH2 enzymatic assay obtained by modifying a published protocol, for example by Yang et al. This assay measures an inhibitor-caused fluorescence decrease of the NADH generated from the ALDH2 catalytic reaction. Inhibitor binding causes a decrease in the production of NADH that affects the ability of the enzyme to turnover its substrate aldehyde and decreased the production of carboxylic acid. The assay components include: ALDH2 (50 nM) (Abcam #87415), substrate formaldehyde (300 μM), NAD⁺ (1.2 mM), and testing compound in phosphate buffer (50 mM, pH 7.4) with 0.01% Tween 20 at 25° C. The assay is performed in black 384-well plates (40 L total volume; Costar). The positive control is inhibitor disulfiram (IC₅₀=4.5 μM). The negative control contains the enzyme, substrate solution in phosphate buffer without the addition of the substrate formaldehyde. Measurements are performed 10 min after adding the testing compound by excitation of NAD⁺ at 340 nm, which leads to fluorescence emission at 450 nm. The IC₅₀ values of testing compounds is determined from experiments performed in a concentration-dependent manner of testing compounds by fitting the data to a sigmoidal dose-response equation in GraphPad Prism. All experiments are performed in triplicate.

### ALDH2 Selectivity Against Human ALDH1 and ALDH3.

To obtain inhibitors with ALDH2 selectivity over other closely related isozymes of the ALDH family, testing compounds are counter-screened using optimized ALDH enzymatic assays for both ALDH1 and ALDH3. Briefly, the enzymatic assay developed for ALDH2 is adapted to ALDH1 and ALDH3 isozymes with excitation at 395 shows an emission spectrum with a peak at 420 nm. Compounds that exhibit an TC₅₀ value below 1.0 μM against the ALDH2 and >50 μM in the ALDH1 and ALDH3 selectivity-tests are considered active and selective.

### OATP1-Mediated Uptake.

The OATP1-mediated uptake of synthesized compounds is assessed by an LC-MS-based method employing the OATP1B1- or OATP1B3-expressing HEK293 cells. Briefly, cells are washed twice with uptake buffer to remove floating cells. Compounds diluted in the uptake buffer are replaced in the wells and incubated for 10 min. For screening of compounds at a single concentration and one-time point, the cells are incubated with 100 μM of testing compounds. In each experiment, pitavastatin is used as a positive control. The incubation is stopped by removing the solution and adding 500 L of ice-cold uptake buffer. The cells are washed twice with the buffer. After washing, 200 μL of lysing solution (1:1 MeOH/H2O) containing internal standard is added. The cell plates are shaken with the lysing solution for at least 10 min at room temperature. The cell lysates are then centrifuged at 12,000 rpm for 15 min. Aliquots of the supernatant are transferred to new tubes and drug concentration is quantified by LC-MS/MS. Task 4 yields new YA7068-based ALDH2 inhibitors with specific liver-targeting character.

### Primary Human Hepatocytes Viability Assays.

Initial cell viability assays are performed on the YA7068 and new analogs on primary human hepatocytes (see letter from Prof. Hongbing Wang) using the commercially available CellTiter-Glo® 2.0 (Promega) as described by the manufacturer. The assay based on the amount of ATP and hence live cells allows us to assess the dose-dependent cytotoxicity of the testing compounds. These studies allow us to assess if the compounds are non-toxic for human liver cells.

### Results

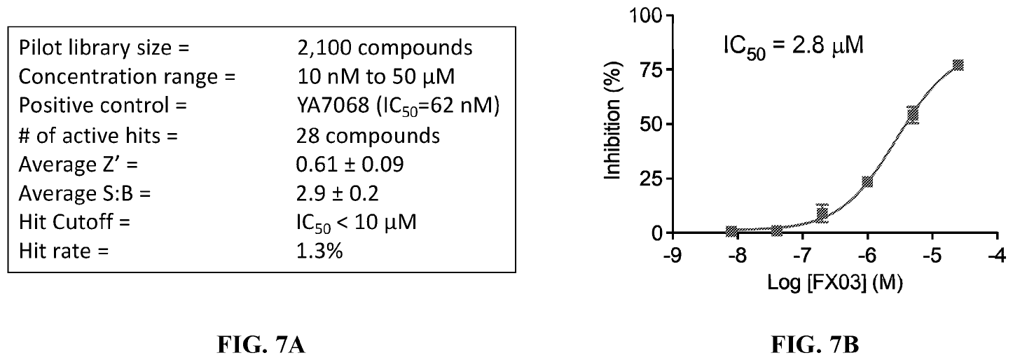

The characterization of YA7068 as a liver-targeted inhibitor of ALDH2 provides a platform for the development of more potent compounds. The medicinal chemistry efforts result in several (4-6) inhibitors with varying degrees of potency. The selection of a lead compound is based on those that showed promising isozyme selectivity and efficient uptake by the liver-specific OATP1 transporters, while not significantly toxic to primary human hepatocytes. A lead compound is subjected to in vivo PK, toxicity, and efficacy studies in mouse AUD models. In a non-limiting example, if there are issues in enhancing inhibitory potency by modification of the YA7068 scaffold, an HTS hit strategy is examined (eg, FX03, FIG. 7). An HTS ALDH2 enzymatic assay is established and can be used to measure the inhibitory potency of compounds. Compounds that bind to ALDH2 (either to the active site or allosteric site), thereby decreasing the fluorescence signal of the NADH generated from the enzymatic reaction, are rapidly identified, and subsequently used as hits for the development of anti-AUD agents. The assay was used for temperature, pH value, and concentration of ALDH2 in the 384-well format; several independent test runs were performed to assess assay parameters. For example, the concentration of the enzyme was determined after performing dose-response experiments by comparing the window of activity looking for a maximal response using disulfiram as a control. The assay has been used to test compounds. Consistent with excellent assay performance in the 384-well format, the Selleck Bioactive collection pilot runs displayed acceptable signal-to-background ratio (S:B; 2.9±0.2), Z′ values (0.61±0.09), with an acceptable hit rate (1.3%) (FIG. 7A).

To determine the performance of the established ALDH2 activity assay under HTS conditions, a pilot screen of 980 compounds was performed. Compounds were tested in triplicate at concentrations ranging from 10 nM to 50 μM (0.1% DMSO). Raw assay data were analyzed using DataWarrior software. The IC₅₀ value of each compound was calculated. “High Control” represents wells from the same plate containing ALDH2 (50 nM), formaldehyde (300 μM), and NAD⁺ (1.2 mM) in phosphate buffer (50 mM, pH 7.4) with 0.01% Tween 20 at 25° C. and DMSO (0.1%) while “Low controls” correspond to the wells from the same plate containing ALDH2 (50 nM) treated with and NAD⁺ (1.2 mM) in phosphate buffer (50 mM, pH 7.4) with 0.01% Tween 20 at 25° C. and DMSO (0.1%). Samples with activity greater than three standard deviations above High Control or less than three standard deviations below Low Control were excluded from further calculations. Two values were subsequently calculated: 1) the average IC₅₀ values of all the compounds tested in the screen, and 2) three times the standard deviation. The sum of these two values was used as the cutoff parameter. Average Z′ across all plates was 0.61±0.09 with hit rates of 1.3%. As an example, hit FX03 indicated an IC₅₀ value of 2.8 μM (FIG. 6B). These data indicate the assay meets HTS criteria, is able to identify an appropriate number of “hits” as potential inhibitors of ALDH2.

### Test the Pharmacological Consequences of Liver-Specific Inhibition of ALDH2.

The efficacy of a lead compound is tested for its PK/Toxicity profile and the in vivo efficacy in the AUD mouse models.

### PK Study.

The drug clearance rate, volume of distribution, Cₘₐₓ, and in vivo half-life of the top compound identified are determined. These studies allow the estimate of the time needed to reach steady state plasma concentration. Based on experience with disulfiram, initial studies involve a single i.v. dose at 50 mg/kg body weight [C57BL/6 outbred mice (n=30), 9 time points & 1 control, 3 mice each]. Mice are euthanized at 5, 15, and 30 min, and at 1, 2, 4, 6, 12 and 24 h. An LC/MS method is utilized to detect YA7068 at the nanomolar level. Heart, liver, kidney, lung, and brain are collected for determination of compound bioavailability in each organ. Dose and time points are altered for the subsequent two studies. For clearance kinetics, a t_{1/2} is 3-4 h or longer. In some embodiments, a multiple dosing protocol is used to achieve the desired steady state plasma concentration within 14 h. Once the range of clinically-relevant plasma concentrations are established, a first estimate of bioavailability for oral dosing is ade. Task 6 results in a top compound with plasma clearance (CL) of less than 30% of blood flow, a half-life (t1/2) more than 3.5 h, and a distribution volume more than 0.75 L/kg.

### Establish Maximum Tolerated Doses (MTDs).

This study of a lead compound provides the estimate of the MTDs and therapeutic window. C57BL/6 mice are treated by a single i.v. injection of the top compound. 20 mice form four groups (5 mice/group; 3 dose levels of testing compound and one vehicle control). Compound dosages are set by PK study results and employ half-log intervals. Mice are weighed and observed for two weeks, then euthanized (earlier if they show 20% weight loss or signs of distress). Metabolic monitoring using a comprehensive lab animal monitoring system (CLAMS) is performed to assess basal metabolism, cage movement, and food intake. Upon sacrifice, liver, heart, kidney, lung, and brain tissue are formalin fixed. Tissues are embedded, sectioned, and hematoxylin & eosin stained then evaluated by a trained observer for tissue damage. Completion yields a compound with the MTDs >500 mg/kg.

### In Vivo Efficacy of the Top Compound in Acetaldehyde Metabolism and in Mouse Model of AUD.

The efficacy of a lead compound in acetaldehyde metabolism is determined in vivo. Briefly, mice are treated with the top compound or vehicle for several time points, followed by orally administrating ethanol (1 g to 5 g ethanol/kg body weight). The blood samples are collected for acetaldehyde measurement. In a non-limiting example, if the compound effectively reduces acetaldehyde metabolism, hepatocyte-specific Aldh2 knockout mice are used to test whether these effects are liver specific. In a non-limiting example, if the inhibitory effect of the compound is liver specific, its effect should be abolished in hepatocyte-specific Aldh2 knockout mice.

After a compound that specifically inhibits acetaldehyde metabolism in the liver is identified, its anti-AUD effects are tested in mouse models by using two-bottle choice and drinking-in-dark experiments. Briefly, mice are treated with top effective compound or vehicle for several time points, and then subjected to two-bottle choice or drinking-in-dark experiments. For the drinking-in-dark experiments, blood is collected in the morning for acetaldehyde measurements. These studies determine whether a lead compound effectively reduces drinking preference and amounts.

In a non-limiting example, if the tested candidate fails to show desirable PK/Tox profile or in vivo efficacy, an alternate compound will be examined. Completion of these studies is expected to identify a lead candidate for the treatments of AUDs.

Exemplary ALDH2 inhibitors of the disclosure are shown in FIG. 8.

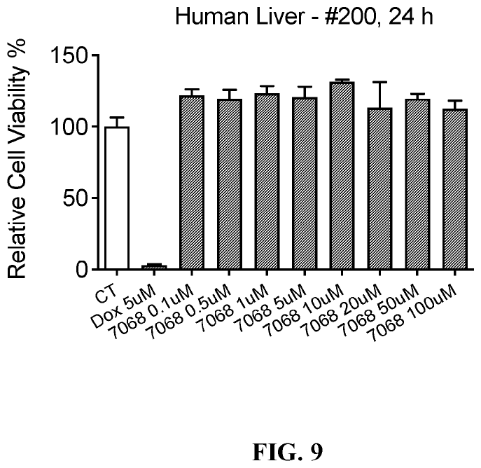

Cytotoxicity studies of YA7068 to primary human hepatocytes were performed. YA7068 did not exhibit any cytotoxicity for primary human hepatocytes with the concentration up to 200 uM (FIG. 9).

### Scientific Rigor.

All synthesized compounds are characterized by ¹H- and ¹³C-NMR along with HRMS, with the HPLC purity >95%. Enzymatic assays and viability assays are performed on three technical replicates. MS instrumentation sensitivity is determined by running internal standards to assess day to day variation. Verification of instrumentation sensitivity is determined by running internal standards as a control to assess day to day variation of instrumentation. All SMPs and inhibitors are verified by NMR and high accuracy mass spectrometry prior to use.

### Methods:

### Cell Viability Assay.

Human primary hepatocytes were maintained in DMEM medium with 10% fetal bovine serum (FBS) and 1% PS (penicillin/streptomycin). The cells were incubated in 37° C. incubator with 5% CO₂. The number of viable cells was determined using the CCK-8 assay. Briefly, cells were seeded at a density of 5000 cells/well in 96-well plates, a nd were treated in triplicate with different concentrations of individual compounds (0, 0.1, 0.5, 1, 5, 10, 25, 50, 100, and 200 PM) for 24 h. Then, 10 μL of CCK-8 working solution was added to each well and incubated for appropriate times (0.5-4 h) before subjecting to a microplate reader for OD detection at 450 nm.

### Synthesis and Characterization of Compounds of the Disclosure

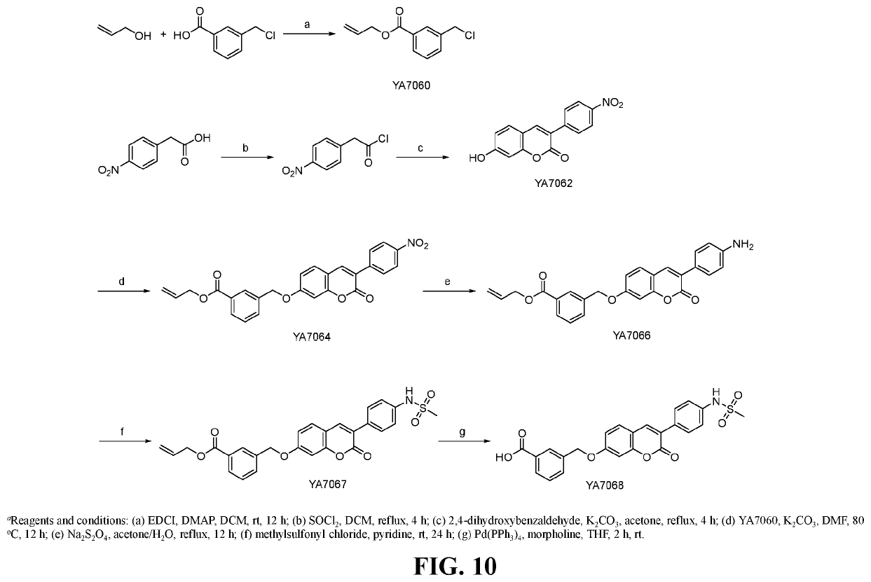

The synthesis of ALDH2 inhibitor YA7068 is detailed in FIG. 10. Allylation of 3-(chloromethyl)benzoic acid using allyl alcohol in the presence of 1-(3-(dimethylamino) propyl)-3-ethylcarbodiimide hydrochloride (EDCI) and 4-dimethylamino-pyridine (DMAP) produced allyl 3-(chloromethyl)benzoate (YA7060). In parallel, the carboxylic acid group of 2-(4-nitrophenyl)acetic acid reacted with thionyl chloride (SOCl₂), and the resulting acyl chloride intermediate was allowed to react with 2,4-dihydroxy benzaldehyde to generate the cyclic product YA7062. Nucleophilic substitution of the chlorine by the phenol group using K2CO3 as a base in dimethylformamide (DMF) afforded compound YA7064. The nitro group in compound YA7064 was subsequently reduced by Na2ₛ2O₄ in acetone/H₂O to provide amino compound YA7066 in moderate yields. Compound YA7066 was treated with methylsulfonyl chloride (MsCl) in pyridine to provide the compound YA7067 in good yields. Finally, the allyl protecting-group of YA7067 was removed using Pd(PPh₃)₄ to yield carboxylic acid-containing compound YA7068 in good yields.

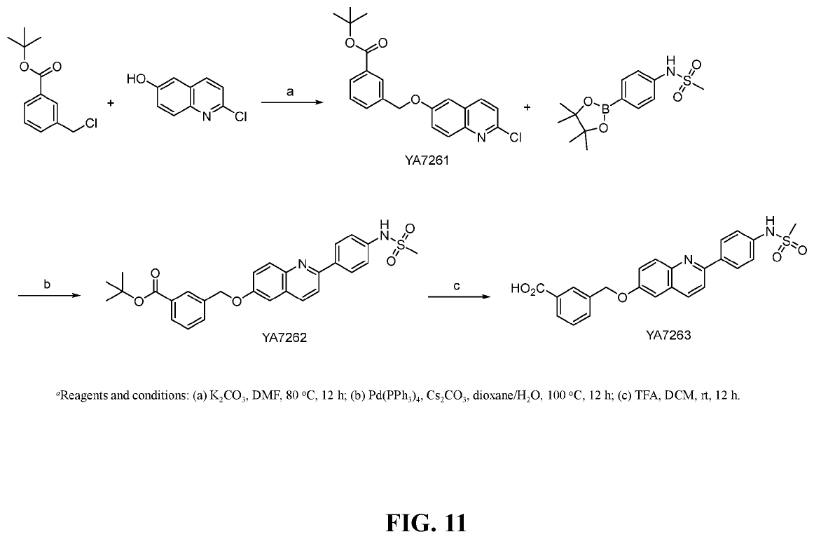

The synthesis of compound YA7263 is shown in FIG. 11. First, tert-butyl 3-(chloromethyl)benzoate was treated with 2-chloroquinolin-6-ol using K₂CO₃ as a base in DMF to provide compound YA7261 in good yields. Next, the Pd-catalyzed cross-coupling of chloro-phenyl compound YA7261 with N-(4-(4,4,5,5-tetramethyl-1,3,2-dioxaborolan-2-yl)phenyl)methanesulfonamide using Cs₂CO₃ as a base under heating yielded product compound YA7262 in moderate yields. Finally, the tert-butyl protecting-group of compound YA7262 was removed using trifluoroacetic acid (TFA) to yield carboxylic acid-containing compound YA7263 in quantitative yields.

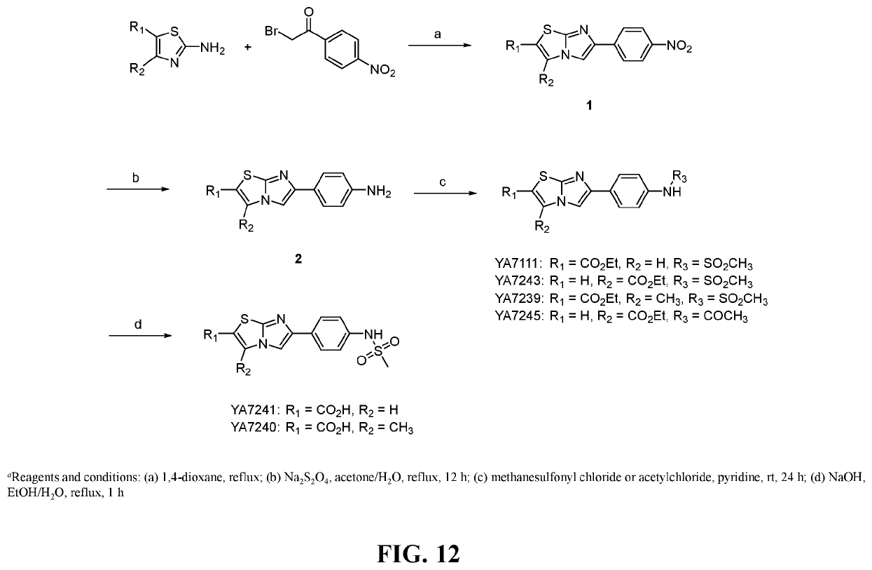

The synthesis of imidazo[2,1-b]thiazoles is detailed in FIG. 12. Treatment of substituted 2-aminothiazole with 2-bromo-1-phenylethan1-one under heating provided cyclic products 1. Reduction of the nitro group in compounds 1 using Na2S₂O₄ in acetone/H₂O generated anilines 2. Next, the amino group of compound 2 was allowed to react with either methanesulfonyl chloride or acetyl chloride in pyridine to provide compounds YA7111, YA7243, YA7239 and YA7245 in good yields. Finally, hydrolysis of the ester groups of YA7111 and YA7239 using NaOH in EtOH/H₂O yielded carboxylic acid-containing compounds YA7241 and YA7240, respectively, in quantitative yields.

### Characterization of Compounds

All chemicals were obtained from commercial suppliers and used without further purification. Analytical thin layer chromatography was visualized by ultraviolet light at 254 nM. ¹H NMR spectra were recorded on a Varian (400 MHz or 500 MHz) spectrometer. Data are presented as follows: chemical shift (in ppm on the δ scale relative to 6=0.00 ppm for the protons in tetramethylsilane (TMS)), integration, multiplicity (s=singlet, d=doublet, t=triplet, q=quartet, m=multiplet, br=broad), coupling constant (J/Hz). ¹³C NMR spectra were recorded at 100 MHz or 125 MHz, and all chemical shifts values are reported in ppm on the 6 scale with an internal reference of δ 77.0 or 39.0 for CDCl₃ or DMSO-d₆, respectively. The purities of title compounds were determined by analytic HPLC, performed on an Agilent 1100 instrument and a reverse-phase column (Waters XTerrra RP18, 5 μM, 4.6×250 mm). All compounds were eluted with 60% acetonitrile/40 water (containing 0.1% TFA) over 20 mins with a detection at 260 nM and a flow rate at 1.0 mL/min. All tested compounds were >95% pure.

### General Procedure A: Synthesis of Compound YA7068

To a solution of 3-(chloromethyl)benzoic acid (1 mmol) in dry CH₂Cl₂ (5 mL), allyl alcohol (1 mmol), 1-(3-(dimethylamino)propyl)-3-ethylcarbodimide hydrochloride (EDCI) (2 mmol), and 4-dimethylaminopyridine (DMAP) (0.2 mmol) were added, and the mixture was stirred at room temperature for 12 h. The reaction solution was diluted with water and extracted with CH₂Cl₂. The organic layer was washed with brine and then dried with sodium sulfate, filtered, and evaporated in vacuo to give corresponding crude compound YA7060, which was used in the next without further purification.

To a solution of 2-(4-nitrophenyl)acetic acid (1 mmol) in dry CH₂C₁₂ (5 mL), thionyl chloride (10 mmol) was added, and the mixture was reflux for 4 h. The resulting solution was evaporated, and the residue was dissolved in dry acetone (50 mL) and 2,4-dihydroxybenzaldehyde (1 mmol) was added. The mixture was refluxed with anhydrous K2CO₃ (4 mmol) for 4 h. Acetone was removed under reduced pressure and cold water (100 mL) was added; 3 N HCl was then added until the solution became acidic. The resulting precipitate was filtered, washed with water and recrystallized from ethanol to give the desired product YA7062.

A mixture of 7-hydroxy-3-(4-nitrophenyl)-2H-chromen-2-one (YA7262, 1 mmol), allyl 3-(chloromethyl)benzoate (YA7060, 1.1 mmol) and potassium carbonate (1.2 mmol) in anhydrous N,N-dimethylformamide (2 ml) was heated with stirring under nitrogen at 80° C. for 12 hours. After cooling to room temperature, the mixture was quenched with about 20 ml of water, and stirred for 30 minutes. The precipitate formed was filtered off, washed three times with water, and dried under vacuum to provide crude product YA7064.

A suspension of allyl 3-(((3-(4-nitrophenyl)-2-oxo-2H-chromen-7-yl)oxy)methyl)benzoate (YA7064, 1 mmol), and sodium dithionite (10 mmol) in acetone (8 ml) and water (4 ml) was reflux for 12 hour. The reaction solution was evaporated in vacuo and the resulting crude product was purified by flash chromatography on silica gel column (methylene chloride/methanol 99.5/0.5 v/v) to provide compound YA7066.

To a solution of allyl 3-(((3-(4-aminophenyl)-2-oxo-2H-chromen-7-yl)oxy)methyl)benzoate YA7066 (1 mmol) in pyridine (5 mL), methylsulfonyl chloride (1.2 mmol) was added, and the mixture was reflux for 24 h. The reaction solution was evaporated in vacuo and the resulting crude product was purified by flash chromatography on silica gel column (methylene chloride/methanol 95/5v/v) to provide compound YA7067.

To a solution of allyl 3-(((3-(4-(methylsulfonamido)phenyl)-2-oxo-2H-chromen-7-yl)oxy)methyl)benzoate (YA7067, 1 mmol), tetrakis(triphenyl-phosphine)palladium(0) (Pd(PPh₃)₄, 0.05 mmol) in dry tetrahydrofuran (2 ml) was added morpholine (5 mmol), and the mixture was stirred at room temperature under nitrogen for 2 hours. The reaction solution was then evaporated in vacuo and the resulting crude product was purified by flash chromatography on silica gel column (methylene chloride/methanol 95/5 v/v containing 1% acetic acid) to provide the title compound YA7068.

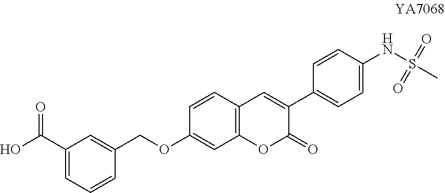 [SMILES: CS(=O)(=O)Nc1ccc(-c2cc3ccc(OCc4cccc(C(=O)O)c4)cc3oc2=O)cc1]

3-(((3-(4-(Methylsulfonamido)phenyl)-2-oxo-2H-chromen-7-yl)oxy)methyl)benzoic acid (YA7068). The title compound was synthesized according to General Procedure A (11%, white solid): ¹H-NMR (400 MHz, DMSO-d₆) δ 8.12 (s, 1H), 8.01 (s, 1H), 7.88 (d, J=6.8 Hz, 1H), 7.66 (t, J=8.0 Hz, 3H), 7.51 (d, J=6.8 Hz, 1H), 7.38 (t, J=7.2, 7.6 Hz, 1H), 7.27 (d, J=8.4 Hz, 2H), 7.09 (s, 1H), 7.04 (d, J=8.8 Hz, 1H), 5.24 (s, 2H), 3.02 (s, 3H); ¹³C-NMR (100 MHz, DMSO-d₆) δ 170.1, 161.7, 160.4, 155.0, 140.4, 139.0, 138.0, 136.1, 130.6, 130.1, 130.0, 129.7, 129.3, 129.0, 128.3, 123.1, 119.5, 113.7, 101.6, 66.0, 44.7; HRMS [M-H]⁻ (ESI-TOF) calcd for C₂₄H₁₈NO₇S 464.0804, found 464.0807.

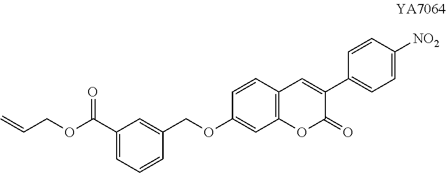 [SMILES: C=CCOC(=O)c1cccc(COc2ccc3cc(-c4ccc([N+](=O)[O-])cc4)c(=O)oc3c2)c1]

Allyl 3-(((3-(4-nitrophenyl)-2-oxo-M-chromen-7-yl)oxy)methyl)benzoate (YA7064). The title compound was synthesized according to General Procedure A:

¹H-NMR (400 MHz, DMSO-d₆) δ 8.39 (s, 1H), 8.28 (d, J=8.0 Hz, 2H), 8.09 (s, 1H), 8.01-7.96 (m, 3H), 7.78-7.71 (m, 2H), 7.58 (t, J=8.0 Hz, 1H), 7.15 (s, 1H), 7.10 (d, J=8.0 Hz, 1H), 3.36 (s, 3H).

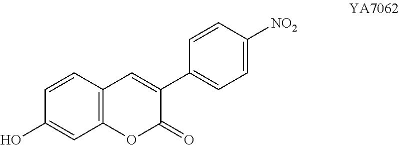 [SMILES: O=c1oc2cc(O)ccc2cc1-c1ccc([N+](=O)[O-])cc1]

7-Hydroxy-3-(4-nitrophenyl)-2H-chromen-2-one (YA7062). The title compound was synthesized according to General Procedure A: ¹H-NMR (400 MHz, DMSO-d₆) δ 10.80 (s, 1H), 8.36 (s, 1H), 8.27 (d, J=8.0 Hz, 2H), 8.00 (d, J=8.0 Hz, 2H), 7.63 (d, J=8.0 Hz, 1H), 6.85 (d, J=8.0 Hz, 1H), 6.75 (s, 1H); ¹³C-NMR (100 MHz, DMSO-d₆) δ 162.6, 160.0, 155.8, 147.0, 143.6, 142.3, 131.1, 129.7, 123.8, 120.2, 114.2, 112.2, 102.2.

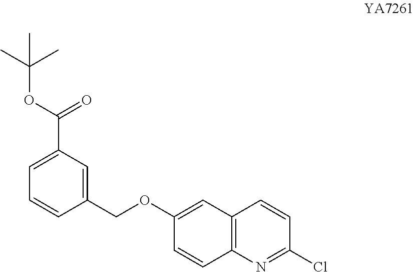 [SMILES: CC(C)(C)OC(=O)c1cccc(COc2ccc3nc(Cl)ccc3c2)c1]

tert-Butyl 3-(((2-chloroquinolin-6-yl)oxy)methyl)benzoate (YA7261). The title compound was synthesized according to General Procedure A (white solid): ¹H-NMR (500 MHz, CDCl₃) δ 8.08 (s, 1H), 7.97 (d, J=7.5 Hz, 1H), 7.90 (t, J=8.5, 9.0 Hz, 2H), 7.61 (d, J=8.0 Hz, 1H), 7.45-7.41 (m, 2H), 7.27 (d, J=8.5 Hz, 1H), 7.09 (d, J=2.5 Hz, 1H), 5.15 (s, 2H), 1.59 (s, 9H); ¹³C-NMR (125 MHz, CDCl₃) δ 165.5, 157.1, 148.3, 144.0, 137.8, 136.6, 132.6, 131.6, 130.2, 129.4, 128.8, 128.6, 127.9, 123.4, 122.7, 106.8, 81.4, 70.0, 28.3;

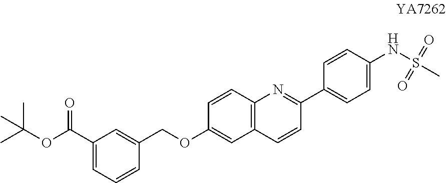 [SMILES: CC(C)(C)OC(=O)c1cccc(COc2ccc3nc(-c4ccc(NS(C)(=O)=O)cc4)ccc3c2)c1]

tert-Butyl 3-(((2-(4-(methylsulfonamido)phenyl)quinolin-6-yl)oxy)methyl)benzoate (YA7262). The title compound was synthesized according to General Procedure A (white solid): ¹H-NMR (500 MHz, CDCl₃) δ 8.12-7.98 (m, 6H), 7.74-7.71 (m, 1H), 7.66 (d, J=6.5 Hz, 1H), 7.46 (t, J=7.5, 9.0 Hz, 2H), 7.38 (d, J=6.5 Hz, 2H), 7.13 (s, 1H), 5.18 (s, 2H), 3.04 (d, J=2.0 Hz, 3H), 1.62 (s, 9H); ¹³C-NMR (125 MHz, CDCl₃) δ 165.8, 156.8, 154.3, 144.5, 138.0, 136.9, 136.7, 135.9, 132.6, 131.7, 131.3, 129.4, 128.8, 128.6, 128.2, 122.9, 120.6, 119.1, 106.6, 81.5, 69.9, 39.5, 28.4;

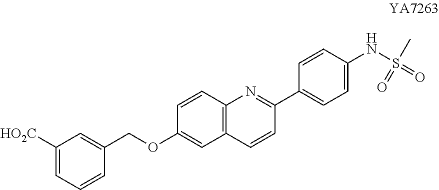 [SMILES: CS(=O)(=O)Nc1ccc(-c2ccc3cc(OCc4cccc(C(=O)O)c4)ccc3n2)cc1]

3-(((2-(4-(Methylsulfonamido)phenyl)quinolin-6-yl)oxy)methyl)benzoic acid (YA7263). The title compound was synthesized according to General Procedure A (white solid): ¹H-NMR (500 MHz, CDCl₃) δ 13.05 (s, 1H), 10.01 (s, 1H), 8.30 (d, J=8.5 Hz, 1H), 8.21 (d, J=8.0 Hz, 2H), 8.11 (s, 1H), 8.04 (d, J=8.5 Hz, 1H), 7.98-7.93 (m, 2H), 7.77 (d, J=8.0 Hz, 1H), 7.56-7.48 (m, 3H), 7.37 (d, J=9.0 Hz, 2H), 5.32 (s, 2H), 3.07 (s, 3H); ¹³C-NMR (125 MHz, CDCl₃) δ 170.3, 159.2, 156.4, 146.7, 142.6, 140.4, 139.1, 137.1, 135.2, 134.2, 133.7, 132.0, 131.6, 131.1, 130.9, 125.6, 122.4, 121.8, 110.2, 72.2, 42.3;

### General Procedure for the Synthesis of Compound 1 (FIG. 12)

To a solution of ethyl 2-aminothiazole-4-carboxylate (10 mmol) in 1,4-dioxane (20 mL) was added 2-bromoacetophenone (10 mmol) at room temperature. The reaction mixture was heated under reflux for 12 h. The mixture was cooled to room temperature and filtered through Celite. The separated hydrobromide was washed with cold ethanol (50 mL), and dried to afford compound 1.

### General Procedure for the Synthesis of Compound 2 (FIG. 12)

A suspension of compound 1 (1 mmol) and sodium dithionite (10 mmol) in acetone (8 mL) and water (4 mL) was heated under reflux for 12 h, and then cooled back to room temperature. The reaction mixture was evaporated in vacuo and the resulting crude product was purified by flash chromatography on silica gel column (DCM/methanol 99.5/0.5 v/v) to provide compound 2.

### General Procedure for the Synthesis of YA7111, YA7243, YA7239 and YA7245.

To a solution of compound 2 (1 mmol) in pyridine (10 mL) was added methanesulfonyl chloride or acetyl chloride (1.1 mmol) dropwise at 0° C. Afterwards the reaction mixture was allowed to warm to room temperature and stirred for an additional 24 h. The solvent was removed by rotary evaporation and the crude product was purified by silica gel column chromatography to obtain YA7111, YA7243, YA7239 and YA7245.

### General Procedure for the Synthesis of YA7241 and YA7240

To a solution of YA7111 or YA7239 (1 mmol) in EtOH (10 mL) was added aqueous NaOH (2M, 10 mL). The reaction mixture was heated under reflux for 1 h and then cooled back to room temperature. The solvents were removed by rotary evaporation and the resulting solution was acidified using HCl (2M) to pH below 2. The white precipitation was collected by filtration and dried to yield the carboxylic acid-containing compounds YA7111 or YA7239.

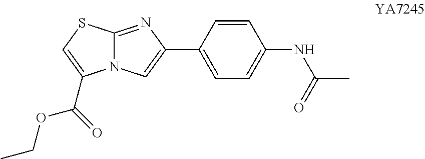 [SMILES: CCOC(=O)c1csc2nc(-c3ccc(NC(C)=O)cc3)cn12]

Ethyl 6-(4-acetamidophenyl)imidazo[2,1-b]thiazole-3-carboxylate (YA7245). The title compound was synthesized according to General Procedure A (white solid):

¹H-NMR (500 MHz, DMSO-d₆) δ 10.13 (s, 1H), 8.46 (s, 1H), 8.29 (s, 1H), 7.84 (d, J=7.0 Hz, 2H), 7.67 (d, J=7.5 Hz, 2H), 4.44-4.38 (m, 2H), 2.06 (s, 3H), 1.37 (t, J=7.0, 4.5 Hz, 3H); ¹³C-NMR (125 MHz, DMSO-d₆) δ 173.8, 162.4, 152.8, 147.4, 145.1, 131.7, 131.1, 129.9, 128.9, 124.4, 115.6, 67.5, 29.4, 19.4;

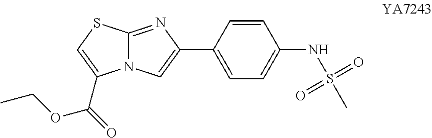 [SMILES: CCOC(=O)c1csc2nc(-c3ccc(NS(C)(=O)=O)cc3)cn12]

Ethyl 6-(4-(methylsulfonamido)phenyl)imidazo[2,1-b]thiazole-3-carboxylate (7243). The title compound was synthesized according to General Procedure A (white solid): ¹H-NMR (500 MHz, CDCl₃) δ 9.79 (s, 1H), 8.38 (s, 1H), 8.20 (s, 1H), 7.86 (d, J=8.0 Hz, 2H), 7.25 (d, J=7.5 Hz, 2H), 4.41 (q, J=7.0, 6.5 Hz, 2H), 3.01 (s, 3H), 1.36 (t, J=7.0, 7.5 Hz, 3H); ¹³C-NMR (125 MHz, CDCl₃) δ 160.6, 151.3, 149.4, 140.6, 132.7, 129.0, 127.4, 126.1, 123.1, 113.0, 64.9, 42.4, 17.2;

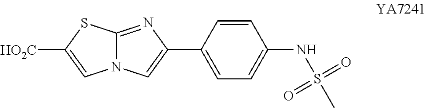 [SMILES: CS(=O)(=O)Nc1ccc(-c2cn3cc(C(=O)O)sc3n2)cc1]

6-(4-(Methylsulfonamido)phenyl)imidazo[2,1-b]thiazole-2-carboxylic acid (YA7241). The title compound was synthesized according to General Procedure A; white solid): ¹H-NMR (500 MHz, DMSO-d₆) δ 9.82 (s, 1H), 8.66 (s, 1H), 8.18 (s, 1H), 7.83 (d, J=8.5 Hz, 2H), 7.25 (d, J=8.5 Hz, 2H), 3.01 (s, 3H); ¹³C-NMR (125 MHz, DMSO-d₆) δ 167.8, 154.8, 152.4, 143.1, 134.5, 131.6, 131.4, 126.7, 125.2, 114.8, 45.2;

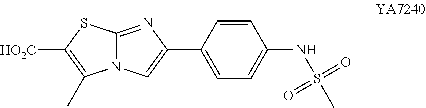 [SMILES: Cc1c(C(=O)O)sc2nc(-c3ccc(NS(C)(=O)=O)cc3)cn12]

3-Methyl-6-(4-(methylsulfonamido)phenyl)imidazo[2,1-b]thiazole-2-carboxylic acid (YA7240). The title compound was synthesized according to General Procedure A (white solid): ¹H-NMR (500 MHz, DMSO-d₆) δ 9.81 (s, 1H), 8.30 (d, J=9.0 Hz, 1H), 7.82 (d, J=5.0 Hz, 2H), 7.25 (d, J=7.0 Hz, 2H), 3.02 (s, 3H), 2.74 (s, 3H); ¹³C-NMR (125 MHz, DMSO-d₆) δ 168.6, 152.2, 143.0, 141.7, 134.7, 131.3, 125.2, 119.9, 113.4, 45.2, 18.0;

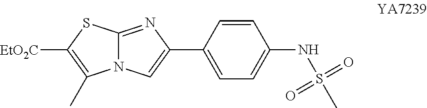 [SMILES: CCOC(=O)c1sc2nc(-c3ccc(NS(C)(=O)=O)cc3)cn2c1C]

Ethyl 3-methyl-6-(4-(methylsulfonamido)phenyl)imidazo[2,1-b]thiazole-2-carboxylate (YA7239). The title compound was synthesized according to General Procedure A (white solid): ¹H-NMR (500 MHz, DMSO-d₆) δ 9.82 (s, 1H), 8.36 (s, 1H), 7.83 (d, J=8.0 Hz, 2H), 7.25 (d, J=7.5 Hz, 2H), 4.32 (q, J=6.5, 7.0 Hz, 2H), 3.02 (s, 3H), 2.77 (s, 3H), 1.31 (t, J=6.5, 7.0 Hz, 3H); ¹³C-NMR (125 MHz, DMSO-d₆) δ 166.9, 152.4, 152.1, 143.1, 142.5, 134.5, 131.2, 125.1, 118.2, 113.4, 66.8, 45.2, 19.4, 18.2;

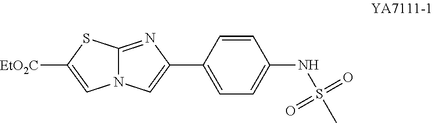 [SMILES: CCOC(=O)c1cn2cc(-c3ccc(NS(C)(=O)=O)cc3)nc2s1]

Ethyl 6-(4-(methylsulfonamido)phenyl)imidazo[2,1-b]thiazole-2-carboxylate (YA7111-1). The title compound was synthesized according to General Procedure A (white solid): ¹H-NMR (400 MHz, DMSO-d₆) δ 8.76 (s, 1H), 8.45 (s, 1H), 8.22 (d, J=8.4 Hz, 2H), 8.06 (d, J=8.4 Hz, 2H), 4.34 (q, J=6.4, 7.2 Hz, 2H), 1.32 (t, J=6.4 Hz, 3H); ¹³C-NMR (100 MHz, DMSO-d₆) δ 161.1, 146.7, 145.7, 140.2, 127.5, 126.1, 124.5, 121.1, 113.3, 62.4, 14.5;

### REFERENCES

1. Carvalho, A. F.; Heilig, M.; Perez, A.; Probst, C.; Rehm, J., Alcohol use disorders. Lancet 2019, 394 (10200), 781-792.
2. Fuster, D.; Samet, J. H., Alcohol Use in Patients with Chronic Liver Disease. N Engl J Med 2018, 379 (13), 1251-1261.
3. Singhvi, A.; Yadav, D., Myths and realities about alcohol and smoking in chronic pancreatitis. Curr Opin Gastroenterol 2018, 34 (5), 355-361.
4. Djousse, L.; Gaziano, J. M., Alcohol consumption and heart failure: a systematic review. Curr Atheroscler Rep 2008, 10 (2), 117-20.
5. Ward, R. J., Alcoholic brain damage. RSC Metallobiol. 2013, 1 (Mechanisms and Metal Involvement in Neurodegenerative Diseases), 186-213.
6. Sanford, N. N.; Sher, D. J.; Xu, X.; Xu, X.; Ahn, C.; Ahn, C.; D'Amico, A. V.; Aizer, A. A.; Mahal, B. A., Alcohol Use Among Patients With Cancer and Survivors in the United States, 2000-2017. J Natl Compr Canc Netw 2020, 18 (1), 69-79.
7. Hyun, J.; Hyun, J.; Hyun, J.; Han, J.; Lee, C.; Jung, Y.; Yoon, M.; Jung, Y., Pathophysiological Aspects of Alcohol Metabolism in the Liver. Int J Mol Sci 2021, 22 (11).
8. Kubiak-Tomaszewska, G.; Tomaszewski, P.; Pachecka, J.; Struga, M.; Olejarz, W.; Mielczarek-Puta, M.; Nowicka, G., Molecular mechanisms of ethanol biotransformation: enzymes of oxidative and nonoxidative metabolic pathways in human. Xenobiotica 2020, 50 (10), 1180-1201.
9. Wang, Q.; Zou, Z.; Wang, Q.; Chang, B.; Zou, Z.; Li, X., Role of ALDH2 in Hepatic Disorders: Gene Polymorphism and Disease Pathogenesis. J Clin Transl Hepatol 2021, 9 (1), 90-98.
10. Edenberg, H. J.; McClintick, J. N., Alcohol Dehydrogenases, Aldehyde Dehydrogenases, and Alcohol Use Disorders: A Critical Review. Alcohol.: Clin. Exp. Res. 2018, 42 (12), 2281-2297.
11. Chen, C.-H.; Ferreira, J. C. B.; Gross, E. R.; Mochly-Rosen, D., Targeting aldehyde dehydrogenase 2: new therapeutic opportunities. Physiol. Rev. 2014, 94 (1), 1-34.
12. MacDonagh, L.; Gray, S. G.; Cuffe, S.; Finn, S. P.; O'Byrne, K. J.; Barr, M. P.; Gallagher, M. F.; Ffrench, B.; Gasch, C.; O'Leary, J. J.; Breen, E.; Nicholson, S.; Leonard, N.; Finn, S. P.; Ryan, R.; Young, V.; O'Byrne, K. J., Targeting the cancer stem cell marker, aldehyde dehydrogenase 1, to circumvent cisplatin resistance in NSCLC. Oncotarget 2017, 8 (42), 72544-72563.
13. Brooks, P. J.; Enoch, M.-A.; Goldman, D.; Li, T.-K.; Yokoyama, A., The alcohol flushing response: an unrecognized risk factor for esophageal cancer from alcohol consumption. PLoS Med. 2009, 6 (3), No pp. given.
14. Eng, M. Y.; Luczak, S. E.; Wall, T. L., ALDH2, ADH1B, and ADH1C genotypes in Asians: a literature review. Alcohol Res Health 2007, 30 (1), 22-7.
15. Chen, Y.-C.; Peng, G.-S.; Tsao, T.-P.; Wang, M.-F.; Lu, R.-B.; Yin, S.-J., Pharmacokinetic and pharmacodynamic basis for overcoming acetaldehyde-induced adverse reaction in Asian alcoholics, heterozygous for the variant ALDH2*2 gene allele. Pharmacogenet. Genomics 2009, 19 (8), 588-599.
16. Isse, T.; Matsuno, K.; Oyama, T.; Kitagawa, K.; Kawamoto, T., Aldehyde dehydrogenase 2 gene targeting mouse lacking enzyme activity shows high acetaldehyde level in blood, brain, and liver after ethanol gavages. Alcohol.: Clin. Exp. Res. 2005, 29 (11), 1959-1964.
17. Guillot, A.; Ren, T.; Jourdan, T.; Pawlosky, R. J.; Han, E.; Kim, S.-J.; Zhang, L.; Koob, G. F.; Gao, B., Targeting liver aldehyde dehydrogenase-2 prevents heavy but not moderate alcohol drinking. Proceedings of the National Academy of Sciences 2019, 116 (51), 25974-25981.
18. Dinavahi, S. S.; Bazewicz, C. G.; Gowda, R.; Robertson, G. P., Aldehyde Dehydrogenase Inhibitors for Cancer Therapeutics. Trends Pharmacol. Sci. 2019, 40 (10), 774-789.
19. Skinner, M. D.; Lahmek, P.; Pham, H.; Aubin, H. J., Disulfiram efficacy in the treatment of alcohol dependence: a meta-analysis. PLoS One 2014, 9 (2), e87366.
20. Jorgensen, C. H.; Pedersen, B.; Tonnesen, H., The efficacy of disulfiram for the treatment of alcohol use disorder. Alcohol Clin Exp Res 2011, 35 (10), 1749-58.
21. Malcolm, R.; Olive, M. F.; Lechner, W., The safety of disulfiram for the treatment of alcohol and cocaine dependence in randomized clinical trials: guidance for clinical practice. Expert Opin Drug Saf 2008, 7 (4), 459-72.
22. Lowe, E. D.; Gao, G. Y.; Johnson, L. N.; Keung, W. M., Structure of daidzin, a naturally occurring anti-alcohol-addiction agent, in complex with human mitochondrial aldehyde dehydrogenase. J Med Chem 2008, 51 (15), 4482-7.
23. Arolfo, M. P.; Overstreet, D. H.; Yao, L.; Fan, P.; Lawrence, A. J.; Tao, G.; Keung, W. M.; Vallee, B. L.; Olive, M. F.; Gass, J. T.; Rubin, E.; Anni, H.; Hodge, C. W.; Besheer, J.; Zablocki, J.; Leung, K.; Blackburn, B. K.; Lange, L. G.; Diamond, I., Suppression of heavy drinking and alcohol seeking by a selective ALDH-2 inhibitor. Alcohol Clin Exp Res 2009, 33 (11), 1935-44.
24. Chen, L. R.; Ko, N. Y.; Chen, K. H., Isoflavone Supplements for Menopausal Women: A Systematic Review. Nutrients 2019, 11 (11).
25. O'Malley, S. S.; Shram, M. J.; Levy-Cooperman, N.; Vince, B.; Strumph, P. M.; Diamond, I.; Blackburn, B. K., Interaction of Ethanol and Oral ANS-6637, a Selective ALDH2 Inhibitor in Males: A Randomized, Double-Blind, Placebo-Controlled, Single-Ascending Dose Cohort Study. Alcohol Clin Exp Res 2020, 44 (9), 1885-1895.
26. Roth, M.; Obaidat, A.; Hagenbuch, B., OATPs, OATs and OCTs: the organic anion and cation transporters of the SLCO and SLC22A gene superfamilies. Br J Pharmacol 2012, 165 (5), 1260-87.
27. Abe, T.; Kakyo, M.; Tokui, T.; Nakagomi, R.; Nishio, T.; Nakai, D.; Nomura, H.; Unno, M.; Suzuki, M.; Naitoh, T.; Matsuno, S.; Yawo, H., Identification of a novel gene family encoding human liver-specific organic anion transporter LST-1. J Biol Chem 1999, 274 (24), 17159-63.
28. Konig, J.; Cui, Y.; Nies, A. T.; Keppler, D., Localization and genomic organization of a new hepatocellular organic anion transporting polypeptide. J Biol Chem 2000, 275 (30), 23161-8.
29. Niemi, M., Role of OATP transporters in the disposition of drugs. Pharmacogenomics 2007, 8 (7), 787-802.
30. Niemi, M.; Pasanen, M. K.; Neuvonen, P. J., Organic anion transporting polypeptide 1B1: a genetically polymorphic transporter of major importance for hepatic drug uptake. Pharmacol Rev 2011, 63 (1), 157-81.
31. Huard, K.; Smith, A. C.; Cappon, G.; Dow, R. L.; Edmonds, D. J.; El-Kattan, A.; Esler, W. P.; Fernando, D. P.; Griffith, D. A.; Kalgutkar, A. S.; Ross, T. T.; Bagley, S. W.; Beebe, D.; Bi, Y. A.; Cabral, S.; Crowley, C.; Doran, S. D.; Dowling, M. S.; Liras, S.; Mascitti, V.; Niosi, M.; Pfefferkorn, J. A.; Polivkova, J.; Preville, C.; Price, D. A.; Shavnya, A.; Shirai, N.; Smith, A. H.; Southers, J. R.; Tess, D. A.; Thuma, B. A.; Varma, M. V.; Yang, X., Optimizing the Benefit/Risk of Acetyl-CoA Carboxylase Inhibitors through Liver Targeting. J Med Chem 2020, 63 (19), 10879-10896.
32. Pfefferkorn, J. A.; Guzman-Perez, A.; Litchfield, J.; Aiello, R.; Treadway, J. L.; Pettersen, J.; Minich, M. L.; Filipski, K. J.; Jones, C. S.; Tu, M.; Aspnes, G.; Risley, H.; Bian, J.; Stevens, B. D.; Bourassa, P.; D'Aquila, T.; Baker, L.; Barucci, N.; Robertson, A. S.; Bourbonais, F.; Derksen, D. R.; MacDougall, M.; Cabrera, O.; Chen, J.; Lapworth, A. L.; Landro, J. A.; Zavadoski, W. J.; Atkinson, K.; Haddish-Berhane, N.; Tan, B.; Yao, L.; Kosa, R. E.; Varma, M. V.; Feng, B.; Duignan, D. B.; El-Kattan, A.; Murdande, S.; Liu, S.; Ammirati, M.; Knafels, J.; DaSilva-Jardine, P.; Sweet, L.; Liras, S.; Rolph, T. P., Discovery of (S)-6-(3-Cyclopentyl-2-(4-(trifluoromethyl)-1H-imidazol-1-yl)propanamido)nicotinic Acid as a Hepatoselective Glucokinase Activator Clinical Candidate for Treating Type 2 Diabetes Mellitus. Journal of medicinal chemistry 2012, 55 (3), 1318-1333.
33. Lachance, N.; Guiral, S.; Huang, Z.; Leclerc, J. P.; Li, C. S.; Oballa, R. M.; Ramtohul, Y. K.; Wang, H.; Wu, J.; Zhang, L., Discovery of potent and liver-selective stearoyl-CoA desaturase (SCD) inhibitors in an acyclic linker series. Bioorg Med Chem Lett 2012, 22 (1), 623-7.
34. Oballa, R. M.; Belair, L.; Black, W. C.; Bleasby, K.; Chan, C. C.; Desroches, C.; Du, X.; Gordon, R.; Guay, J.; Guiral, S.; Hafey, M. J.; Hamelin, E.; Huang, Z.; Kennedy, B.; Lachance, N.; Landry, F.; Li, C. S.; Mancini, J.; Normandin, D.; Pocai, A.; Powell, D. A.; Ramtohul, Y. K.; Skorey, K.; Sorensen, D.; Sturkenboom, W.; Styhler, A.; Waddleton, D. M.; Wang, H.; Wong, S.; Xu, L.; Zhang, L., Development of a liver-targeted stearoyl-CoA desaturase (SCD) inhibitor (MK-8245) to establish a therapeutic window for the treatment of diabetes and dyslipidemia. J Med Chem 2011, 54 (14), 5082-96.
35. Powell, D. A.; Black, W. C.; Bleasby, K.; Chan, C. C.; Deschenes, D.; Gagnon, M.; Gordon, R.; Guay, J.; Guiral, S.; Hafey, M. J.; Huang, Z.; Isabel, E.; Leblanc, Y.; Styhler, A.; Xu, L. J.; Zhang, L.; Oballa, R. M., Nicotinic acids: liver-targeted SCD inhibitors with preclinical anti-diabetic efficacy. Bioorg Med Chem Lett 2011, 21 (24), 7281-6.
36. Ramtohul, Y. K.; Powell, D.; Leclerc, J. P.; Leger, S.; Oballa, R.; Black, C.; Isabel, E.; Li, C. S.; Crane, S.; Robichaud, J.; Guay, J.; Guiral, S.; Zhang, L.; Huang, Z., Bicyclic heteroaryl inhibitors of stearoyl-CoA desaturase: from systemic to liver-targeting inhibitors. Bioorg Med Chem Lett 2011, 21 (19), 5692-6.
37. Bebernitz, G. R.; Beaulieu, V.; Dale, B. A.; Deacon, R.; Duttaroy, A.; Gao, J.; Grondine, M. S.; Gupta, R. C.; Kakmak, M.; Kavana, M.; Kirman, L. C.; Liang, J.; Maniara, W. M.; Munshi, S.; Nadkarni, S. S.; Schuster, H. F.; Stains, T.; St Denny, I.; Taslimi, P. M.; Vash, B.; Caplan, S. L., Investigation of functionally liver selective glucokinase activators for the treatment of type 2 diabetes. J Med Chem 2009, 52 (19), 6142-52.
38. Guvench, O.; MacKerell Jr, A. D., Computational Fragment-Based Binding Site Identification by Ligand Competitive Saturation. PLoS Comp. Biol. 2009, 5, e1000435.
39. Raman, E. P.; Yu, W.; Guvench, O.; Mackerell, A. D., Reproducing crystal binding modes of ligand functional groups using Site-Identification by Ligand Competitive Saturation (SILCS) simulations. J Chem Inf Model 2011, 51 (4), 877-96.
40. Foster, T. J.; MacKerell, A. D., Jr.; Guvench, O., Balancing target flexibility and target denaturation in computational fragment-based inhibitor discovery. J Comput Chem 2012, 33 (23), 1880-91.
41. Raman, E. P.; Yu, W.; Guvench, O.; Mackerell, A. D., Reproducing crystal binding modes of ligand functional groups using Site-Identification by Ligand Competitive Saturation (SILCS) simulations. J Chem Inf Model 2011, 51 (4), 877-96.
42. Foster, T. J.; MacKerell, A. D., Jr.; Guvench, O., Balancing target flexibility and target denaturation in computational fragment-based inhibitor discovery. J Comput Chem 2012, 33 (23), 1880-91.
43. Guvench, O.; MacKerell, A. D., Jr., Computational fragment-based binding Site Identification by Ligand Competitive Saturation. PLoS Comput. Biol. 2009, 5 (7), No pp. given.
44. Lakkaraju, S. K.; Yu, W.; Raman, E. P.; Hershfeld, A. V.; Fang, L.; Deshpande, D. A.; MacKerell, A. D., Mapping Functional Group Free Energy Patterns at Protein Occluded Sites: Nuclear Receptors and G-Protein Coupled Receptors. J. Chem. Inf. Model. 2015, 55 (3), 700-708.
45. Lakkaraju, S. K.; Raman, E. P.; Yu, W.; MacKerell, A. D., Jr., Sampling of Organic Solutes in Aqueous and Heterogeneous Environments Using Oscillating Excess Chemical Potentials in Grand Canonical-like Monte Carlo-Molecular Dynamics Simulations. Journal of chemical theory and computation 2014, 10 (6), 2281-2290.
46. Cheng, H.; Linhares, B. M.; Yu, W.; Cardenas, M. G.; Ai, Y.; Jiang, W.; Winkler, A.; Cohen, S.; Melnick, A.; MacKerell, A., Jr.; Cierpicki, T.; Xue, F., Identification of Thiourea-Based Inhibitors of the B-Cell Lymphoma 6 BTB Domain via NMR-Based Fragment Screening and Computer-Aided Drug Design. J Med Chem 2018, 61 (17), 7573-7588.
47. He, X.; Lakkaraju, S. K.; Hanscom, M.; Zhao, Z.; Wu, J.; Stoica, B.; MacKerell, A. D., Jr.; Faden, A. I.; Xue, F., Acyl-2-aminobenzimidazoles: A novel class of neuroprotective agents targeting mGluR5. Bioorg. Med. Chem. 2015, 23 (9), 2211-2220.
48. Lakkaraju, S. K.; Mbatia, H.; Hanscom, M.; Zhao, Z.; Wu, J.; Stoica, B.; MacKerell, A. D.; Faden, A. I.; Xue, F., Cyclopropyl-containing positive allosteric modulators of metabotropic glutamate receptor subtype 5. Bioorg. Med. Chem. Lett. 2015, 25 (11), 2275-2279.
49. Heinzl, G. A.; Huang, W.; Yu, W.; Giardina, B. J.; Zhou, Y.; MacKerell, A. D., Jr.; Wilks, A.; Xue, F., Iminoguanidines as Allosteric Inhibitors of the Iron-Regulated Heme Oxygenase (HemO) of Pseudomonas aeruginosa. J Med Chem 2016, 59 (14), 6929-42.
50. Butina, D., Unsupervised data base clustering based on Daylight's fingerprint and Tanimoto similarity: a fast and automated way to cluster small and large data sets. J. Chem. Inf. Comput. Sci. 1999, 39 (4), 747-750.
51. Lipinski, C. A., Drug-like properties and the causes of poor solubility and poor permeability. J. Pharmacol. Toxicol. Methods 2001, 44 (1), 235-249.
52. Pan, Y.; Huang, N.; Cho, S.; MacKerell, A. D., Jr., Consideration of Molecular Weight during Compound Selection in Virtual Target-Based Database Screening. J. Chem. Inf. Comput. Sci. 2003, 43 (1), 267-272.
53. Huang, N.; Nagarsekar, A.; Xia, G.; Hayashi, J.; MacKerell, A. D., Jr., Identification of non-phosphate-containing small molecular weight inhibitors of the tyrosine kinase p56 Lck SH2 domain via in silico screening against the pY+3 binding site. J. Med. Chem. 2004, 47 (14), 3502-3511.
54. Obianom, O. N.; Coutinho, A. L.; Yang, W.; Yang, H.; Xue, F.; Shu, Y., Incorporation of a biguanide scaffold enhances drug uptake by organic cation transporters 1 and 2. Mol. Pharmaceutics 2017, 14 (8), 2726-2739.
55. Yang, S. M.; Yasgar, A.; Miller, B.; Lal-Nag, M.; Brimacombe, K.; Hu, X.; Sun, H.; Wang, A.; Xu, X.; Nguyen, K.; Oppermann, U.; Ferrer, M.; Vasiliou, V.; Simeonov, A.; Jadhav, A.; Maloney, D. J., Discovery of NCT-501, a Potent and Selective Theophylline-Based Inhibitor of Aldehyde Dehydrogenase 1A1 (ALDH1A1). J Med Chem 2015, 58 (15), 5967-78.

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims

1. A compound of Formula (I), or a pharmaceutically acceptable salt, solvate, hydrate, cocrystal, or prodrug thereof: 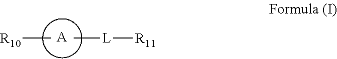
wherein
\textcircled{A} is optionally substituted heteroaryl;
R₁₀ is selected from halo, optionally substituted aryl, optionally substituted heteroaryl;
R₁₁ is selected from —OH, —C(═O)H, —C(═O)OH, —C(═O)OR₁₃, and —C(═O)NH₂;
L is a linker;
R₁₃ is selected from optionally substituted alkyl and optionally substituted alkenyl.

2. The compound of claim 1, wherein L is a bond or —O—(CH₂)ₙ—R₁₄—;
wherein R₁₄ is selected from optionally substituted cycloalkyl, optionally substituted heterocyclyl, optionally substituted aryl, and optionally substituted heteroaryl; and
n is an integer selected from 1, 2, 3, 4, 5, or 6.

3. The compound of claim 1 or 2, wherein
R₁₀ is F, Br, Cl, I, 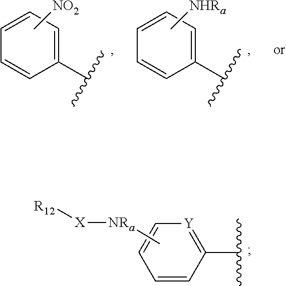
X is selected from —C(═O)— and —S(═O)₂—
Y is selected from CH and N;
R₁₂ is selected from optionally substituted alkyl, optionally substituted cycloalkyl, optionally substituted heterocyclyl, optionally substituted aryl, and optionally substituted heteroaryl; and
Rₐ is selected from H and optionally substituted alkyl.

4. The compound of any one of claims 1-3, wherein:
\textcircled{A} is

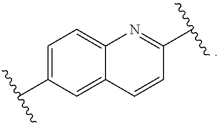 [SMILES: *c1ccc2nc(*)ccc2c1]

5. The compound of claim 4, wherein
L is —O—(CH₂)ₙ—R₁₄—;
R₁₀ is Cl or 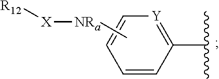
R₁₁ is selected from —C(═O)OH and —C(═O)OR₁₃;
R₁₂ is alkyl; and
R₁₄ is C₆ aryl.

6. The compound of claim 4 or 5, wherein the compound is selected from Formula 1001 to Formula 1008, or a pharmaceutically acceptable salt, solvate, hydrate, cocrystal, or prodrug thereof:

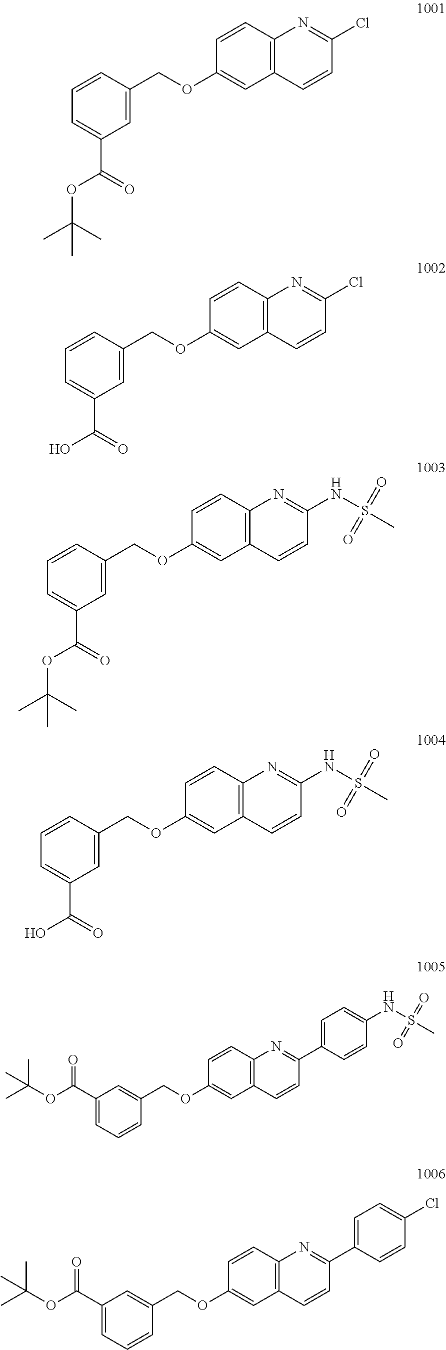

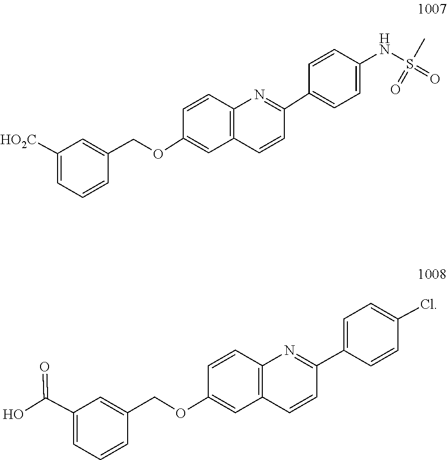 [SMILES: CS(=O)(=O)Nc1ccc(-c2ccc3cc(OCc4cccc(C(=O)O)c4)ccc3n2)cc1 ; O=C(O)c1cccc(COc2ccc3nc(-c4ccc(Cl)cc4)ccc3c2)c1]

7. The compound of claim 4, wherein the compound of Formula (I) is a compound of formula 1007:

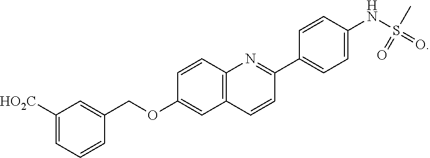 [SMILES: CS(=O)(=O)Nc1ccc(-c2ccc3cc(OCc4cccc(C(=O)O)c4)ccc3n2)cc1]

8. The compound of claim 1, wherein:
\textcircled{A} is

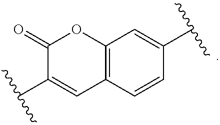 [SMILES: *c1ccc2cc(*)c(=O)oc2c1]

9. The compound of claim 1 or 8, wherein the compound of Formula (I) is a compound of Formula (IIa) Formula (IIb), Formula (IIc), or a pharmaceutically acceptable salt, solvate, hydrate, cocrystal, or prodrug thereof: 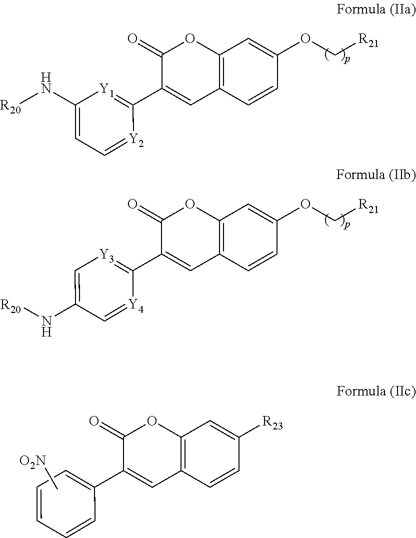
wherein
Y₁ and Y₂ are each CH, Y₁ is CH and Y₂ is N, or Y₁ is N and Y₂ is CH;
Y₃ and Y₄ are each CH, Y₃ is CH and Y₄ is N, or Y₃ is N and Y₄ is CH;
R₂₀ is selected from H, —S(═O)₂R₂₄, and —C(═O)₂R₂₄;
R₂₁ is selected from —C(O)OH, optionally substituted aryl, optionally substituted heteroaryl, and optionally substituted cycloalkyl;
R₂₃ is selected from —OH and 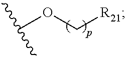
and
R₂₄ is selected from optionally substituted alkyl and optionally substituted cycloalkyl; and
p is an integer selected from 1-14, optionally wherein p is an integer selected from 1-6 or 4-14.

10. The compound of claim 9, wherein R₂₄ is selected from methyl and cyclopropyl.

11. The compound of claim 9 or 10, wherein R₂₀ is selected from H, —S(═O)₂CH₃, —C(═O)CH₃, and

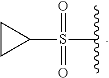 [SMILES: *S(=O)(=O)C1CC1]

12. The compound of any one of claims 9-11, wherein the compound of Formula (IIb) is a compound of Formula (20), or a pharmaceutically acceptable salt, solvate, hydrate, cocrystal, or prodrug thereof:

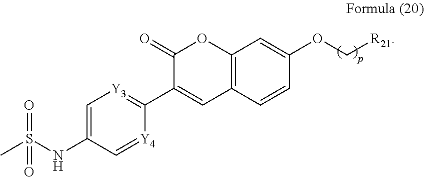

13. The compound of any one of claims 9-12, wherein R₂₁ is selected from optionally substituted phenyl, optionally substituted cyclohexyl, optionally substituted pyridine, and optionally substituted pyrazine.

14. The compound of any one of claims 9-13, wherein R₂₁ is selected from

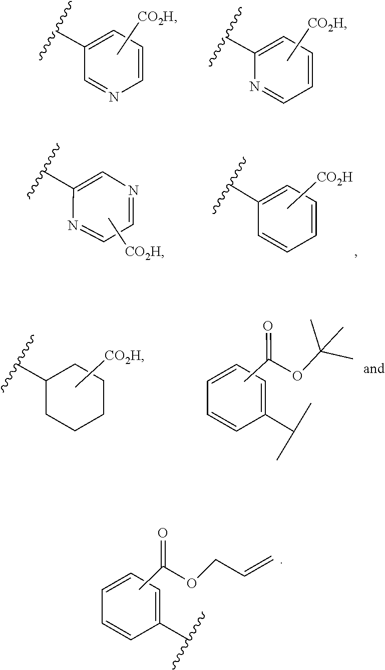

15. The compound of any one of claims 9-14, wherein p is 1.

16. The compound of any one of claims 9-15, wherein Y₁ and Y₂ are each CH.

17. The compound of any one of claims 9-15, wherein Y₃ and Y₄ are each CH.

18. The compound of any one of claims 9-15, wherein Y₃ is N and Y₄ is CH.

19. The compound of any one of claims 9-12, wherein the compound of Formula (IIb) is a compound of Formula (21), or a pharmaceutically acceptable salt, solvate, hydrate, cocrystal, or prodrug thereof:

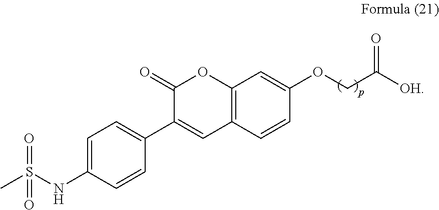

20. The compound of claim 19, wherein p is an integer from 4-14.

21. The compound of claim 9, wherein the compound is of any one of formulas 2001 to 2131, or a pharmaceutically acceptable salt, solvate, hydrate, cocrystal, or prodrug thereof:

| | |
|---|---|
| | Formula (IIa) |
| 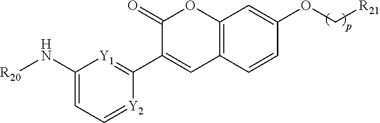 | |
| Compound # | R₂₀ | R₂₁ | Y₁ | Y₂ | p |
|---|---|---|---|---|---|
| 2001 | —S(═O)₂CH₃ | 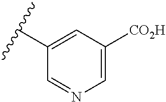 [SMILES: *c1cncc(C(=O)O)c1] | CH | CH | 1 |
| 2002 | —S(═O)₂CH₃ | 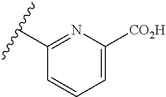 [SMILES: *c1cccc(C(=O)O)n1] | CH | CH | 1 |
| 2003 | —S(═O)₂CH₃ | 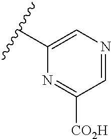 [SMILES: *c1cncc(C(=O)O)n1] | CH | CH | 1 |
| 2004 | —S(═O)₂CH₃ | 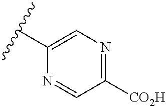 [SMILES: *c1cnc(C(=O)O)cn1] | CH | CH | 1 |
| 2005 | —S(═O)₂CH₃ | 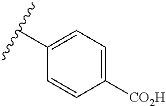 [SMILES: *c1ccc(C(=O)O)cc1] | CH | CH | 1 |
| 2006 | —S(═O)₂CH₃ | 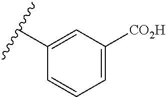 [SMILES: *c1cccc(C(=O)O)c1] | CH | CH | 1 |
| 2007 | —S(═O)₂CH₃ | 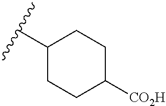 [SMILES: *C1CCC(C(=O)O)CC1] | CH | CH | 1 |
| 2008 | —S(═O)₂CH₃ | 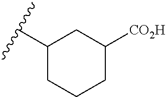 [SMILES: *C1CCCC(C(=O)O)C1] | CH | CH | 1 |
| 2009 | —S(═O)₂CH₃ | 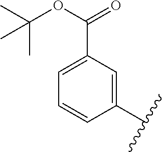 [SMILES: *c1cccc(C(=O)OC(C)(C)C)c1] | CH | CH | 1 |
| 2010 | —S(═O)₂CH₃ | 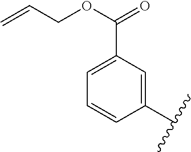 [SMILES: *c1cccc(C(=O)OCC=C)c1] | CH | CH | 1 |
| 2011 | —C(═O)CH₃ | 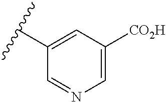 [SMILES: *c1cncc(C(=O)O)c1] | CH | CH | 1 |
| 2012 | —C(═O)CH₃ | 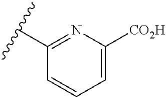 [SMILES: *c1cccc(C(=O)O)n1] | CH | CH | 1 |
| 2013 | —C(═O)CH₃ | 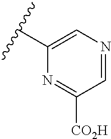 [SMILES: *c1cncc(C(=O)O)n1] | CH | CH | 1 |
| 2014 | —C(═O)CH₃ | 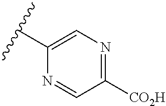 [SMILES: *c1cnc(C(=O)O)cn1] | CH | CH | 1 |
| 2015 | —C(═O)CH₃ | 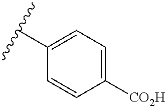 [SMILES: *c1ccc(C(=O)O)cc1] | CH | CH | 1 |
| 2016 | —C(═O)CH₃ | 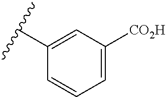 [SMILES: *c1cccc(C(=O)O)c1] | CH | CH | 1 |
| 2017 | —C(═O)CH₃ | 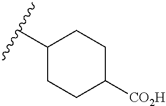 [SMILES: *C1CCC(C(=O)O)CC1] | CH | CH | 1 |
| 2018 | —C(═O)CH₃ | 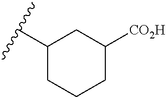 [SMILES: *C1CCCC(C(=O)O)C1] | CH | CH | 1 |
| 2019 | —C(═O)CH₃ | 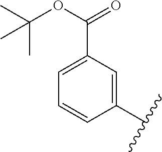 [SMILES: *c1cccc(C(=O)OC(C)(C)C)c1] | CH | CH | 1 |
| 2020 | —C(═O)CH₃ | 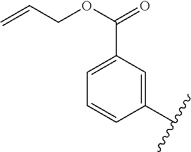 [SMILES: *c1cccc(C(=O)OCC=C)c1] | CH | CH | 1 |
| 2021 | 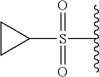 [SMILES: *S(=O)(=O)C1CC1] | 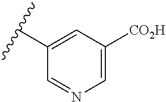 [SMILES: *c1cncc(C(=O)O)c1] | CH | CH | 1 |
| 2022 | 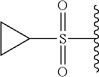 [SMILES: *S(=O)(=O)C1CC1] | 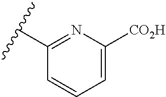 [SMILES: *c1cccc(C(=O)O)n1] | CH | CH | 1 |
| 2023 | 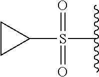 [SMILES: *S(=O)(=O)C1CC1] | 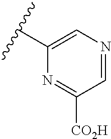 [SMILES: *c1cncc(C(=O)O)n1] | CH | CH | 1 |
| 2024 | 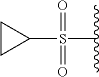 [SMILES: *S(=O)(=O)C1CC1] | 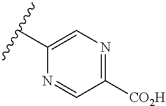 [SMILES: *c1cnc(C(=O)O)cn1] | CH | CH | 1 |
| 2025 | 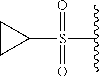 [SMILES: *S(=O)(=O)C1CC1] | 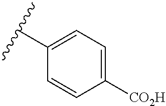 [SMILES: *c1ccc(C(=O)O)cc1] | CH | CH | 1 |
| 2026 | 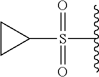 [SMILES: *S(=O)(=O)C1CC1] | 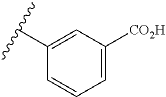 [SMILES: *c1cccc(C(=O)O)c1] | CH | CH | 1 |
| 2027 | 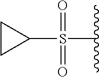 [SMILES: *S(=O)(=O)C1CC1] | 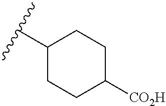 [SMILES: *C1CCC(C(=O)O)CC1] | CH | CH | 1 |
| 2028 | 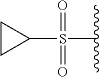 [SMILES: *S(=O)(=O)C1CC1] | 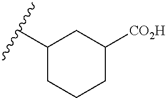 [SMILES: *C1CCCC(C(=O)O)C1] | CH | CH | 1 |
| 2029 | 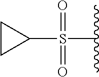 [SMILES: *S(=O)(=O)C1CC1] | 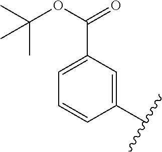 [SMILES: *c1cccc(C(=O)OC(C)(C)C)c1] | CH | CH | 1 |
| 2030 | 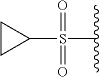 [SMILES: *S(=O)(=O)C1CC1] | 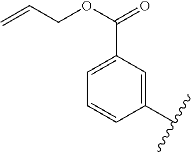 [SMILES: *c1cccc(C(=O)OCC=C)c1] | CH | CH | 1 |
| 2031 | H | 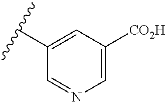 [SMILES: *c1cncc(C(=O)O)c1] | CH | CH | 1 |
| 2032 | H | 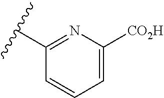 [SMILES: *c1cccc(C(=O)O)n1] | CH | CH | 1 |
| 2033 | H | 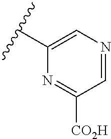 [SMILES: *c1cncc(C(=O)O)n1] | CH | CH | 1 |
| 2034 | H | 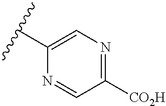 [SMILES: *c1cnc(C(=O)O)cn1] | CH | CH | 1 |
| 2035 | H | 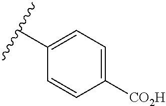 [SMILES: *c1ccc(C(=O)O)cc1] | CH | CH | 1 |
| 2036 | H | 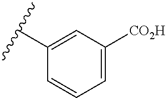 [SMILES: *c1cccc(C(=O)O)c1] | CH | CH | 1 |
| 2037 | H | 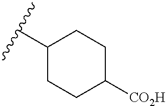 [SMILES: *C1CCC(C(=O)O)CC1] | CH | CH | 1 |
| 2038 | H | 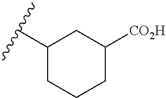 [SMILES: *C1CCCC(C(=O)O)C1] | CH | CH | 1 |
| 2039 | H | 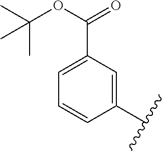 [SMILES: *c1cccc(C(=O)OC(C)(C)C)c1] | CH | CH | 1 |
| 2040 | H | 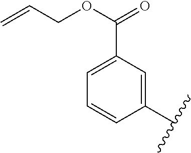 [SMILES: *c1cccc(C(=O)OCC=C)c1] | CH | CH | 1 |
| | |
|---|---|
| | Formula (IIb) |
| 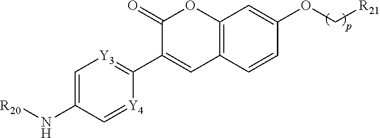 | |
| Compound # | R₂₀ | R₂₁ | Y₃ | Y₄ | p |
|---|---|---|---|---|---|
| 2041 | —S(═O)₂CH₃ | 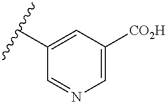 [SMILES: *c1cncc(C(=O)O)c1] | CH | CH | 1 |
| 2042 | —S(═O)₂CH₃ | 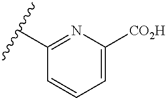 [SMILES: *c1cccc(C(=O)O)n1] | CH | CH | 1 |
| 2043 | —S(═O)₂CH₃ | 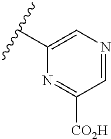 [SMILES: *c1cncc(C(=O)O)n1] | CH | CH | 1 |
| 2044 | —S(═O)₂CH₃ | 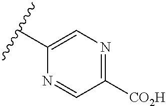 [SMILES: *c1cnc(C(=O)O)cn1] | CH | CH | 1 |
| 2045 | —S(═O)₂CH₃ | 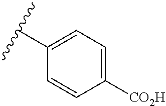 [SMILES: *c1ccc(C(=O)O)cc1] | CH | CH | 1 |
| 2046 | —S(═O)₂CH₃ | 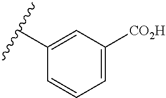 [SMILES: *c1cccc(C(=O)O)c1] | CH | CH | 1 |
| 2047 | —S(═O)₂CH₃ | 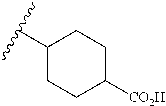 [SMILES: *C1CCC(C(=O)O)CC1] | CH | CH | 1 |
| 2048 | —S(═O)₂CH₃ | 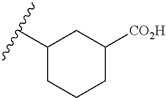 [SMILES: *C1CCCC(C(=O)O)C1] | CH | CH | 1 |
| 2049 | —S(═O)₂CH₃ | 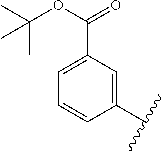 [SMILES: *c1cccc(C(=O)OC(C)(C)C)c1] | CH | CH | 1 |
| 2050 | —S(═O)₂CH₃ | 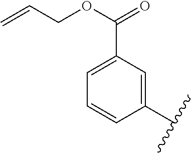 [SMILES: *c1cccc(C(=O)OCC=C)c1] | CH | CH | 1 |
| 2051 | —C(═O)CH₃ | 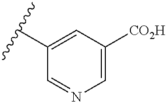 [SMILES: *c1cncc(C(=O)O)c1] | CH | CH | 1 |
| 2052 | —C(═O)CH₃ | 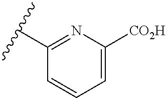 [SMILES: *c1cccc(C(=O)O)n1] | CH | CH | 1 |
| 2053 | —C(═O)CH₃ | 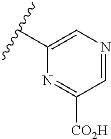 [SMILES: *c1cncc(C(=O)O)n1] | CH | CH | 1 |
| 2054 | —C(═O)CH₃ | 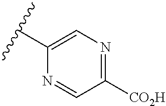 [SMILES: *c1cnc(C(=O)O)cn1] | CH | CH | 1 |
| 2055 | —C(═O)CH₃ | 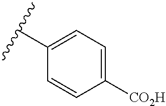 [SMILES: *c1ccc(C(=O)O)cc1] | CH | CH | 1 |
| 2056 | —C(═O)CH₃ | 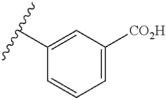 [SMILES: *c1cccc(C(=O)O)c1] | CH | CH | 1 |
| 2057 | —C(═O)CH₃ | 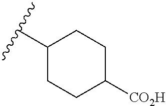 [SMILES: *C1CCC(C(=O)O)CC1] | CH | CH | 1 |
| 2058 | —C(═O)CH₃ | 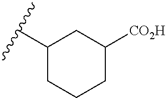 [SMILES: *C1CCCC(C(=O)O)C1] | CH | CH | 1 |
| 2059 | —C(═O)CH₃ | 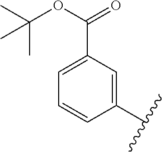 [SMILES: *c1cccc(C(=O)OC(C)(C)C)c1] | CH | CH | 1 |
| 2060 | —C(═O)CH₃ | 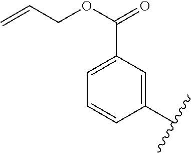 [SMILES: *c1cccc(C(=O)OCC=C)c1] | CH | CH | 1 |
| 2061 | 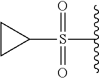 [SMILES: *S(=O)(=O)C1CC1] | 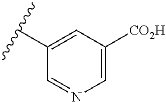 [SMILES: *c1cncc(C(=O)O)c1] | CH | CH | 1 |
| 2062 | 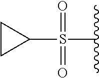 [SMILES: *S(=O)(=O)C1CC1] | 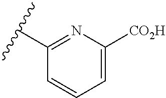 [SMILES: *c1cccc(C(=O)O)n1] | CH | CH | 1 |
| 2063 | 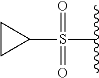 [SMILES: *S(=O)(=O)C1CC1] | 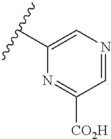 [SMILES: *c1cncc(C(=O)O)n1] | CH | CH | 1 |
| 2064 | 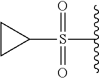 [SMILES: *S(=O)(=O)C1CC1] | 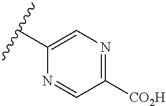 [SMILES: *c1cnc(C(=O)O)cn1] | CH | CH | 1 |
| 2065 | 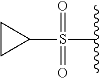 [SMILES: *S(=O)(=O)C1CC1] | 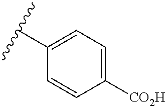 [SMILES: *c1ccc(C(=O)O)cc1] | CH | CH | 1 |
| 2066 | 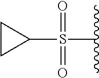 [SMILES: *S(=O)(=O)C1CC1] | 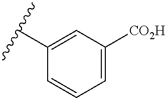 [SMILES: *c1cccc(C(=O)O)c1] | CH | CH | 1 |
| 2067 | 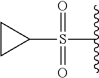 [SMILES: *S(=O)(=O)C1CC1] | 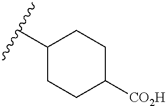 [SMILES: *C1CCC(C(=O)O)CC1] | CH | CH | 1 |
| 2068 | 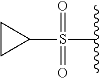 [SMILES: *S(=O)(=O)C1CC1] | 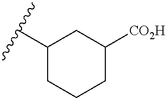 [SMILES: *C1CCCC(C(=O)O)C1] | CH | CH | 1 |
| 2069 | 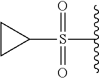 [SMILES: *S(=O)(=O)C1CC1] | 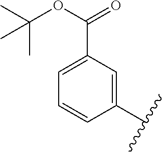 [SMILES: *c1cccc(C(=O)OC(C)(C)C)c1] | CH | CH | 1 |
| 2070 | 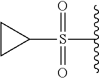 [SMILES: *S(=O)(=O)C1CC1] | 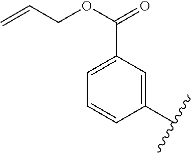 [SMILES: *c1cccc(C(=O)OCC=C)c1] | CH | CH | 1 |
| 2071 | H | 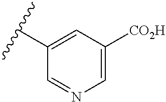 [SMILES: *c1cncc(C(=O)O)c1] | CH | CH | 1 |
| 2072 | H | 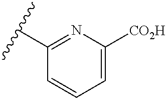 [SMILES: *c1cccc(C(=O)O)n1] | CH | CH | 1 |
| 2073 | H | 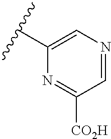 [SMILES: *c1cncc(C(=O)O)n1] | CH | CH | 1 |
| 2074 | H | 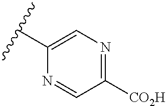 [SMILES: *c1cnc(C(=O)O)cn1] | CH | CH | 1 |
| 2075 | H | 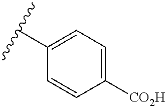 [SMILES: *c1ccc(C(=O)O)cc1] | CH | CH | 1 |
| 2076 | H | 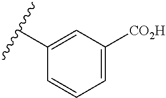 [SMILES: *c1cccc(C(=O)O)c1] | CH | CH | 1 |
| 2077 | H | 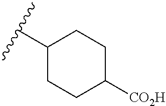 [SMILES: *C1CCC(C(=O)O)CC1] | CH | CH | 1 |
| 2078 | H | 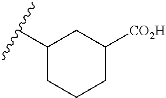 [SMILES: *C1CCCC(C(=O)O)C1] | CH | CH | 1 |
| 2079 | H | 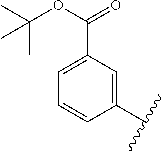 [SMILES: *c1cccc(C(=O)OC(C)(C)C)c1] | CH | CH | 1 |
| 2080 | H | 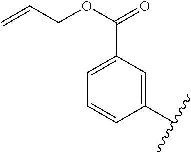 [SMILES: *c1cccc(C(=O)OCC=C)c1] | CH | CH | 1 |
| 2081 | —S(═O)₂CH₃ | 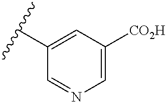 [SMILES: *c1cncc(C(=O)O)c1] | N | CH | 1 |
| 2082 | —S(═O)₂CH₃ | 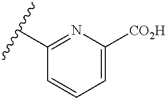 [SMILES: *c1cccc(C(=O)O)n1] | N | CH | 1 |
| 2083 | —S(═O)₂CH₃ | 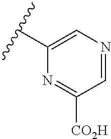 [SMILES: *c1cncc(C(=O)O)n1] | N | CH | 1 |
| 2084 | —S(═O)₂CH₃ | 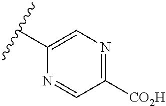 [SMILES: *c1cnc(C(=O)O)cn1] | N | CH | 1 |
| 2085 | —S(═O)₂CH₃ | 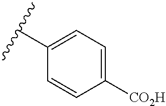 [SMILES: *c1ccc(C(=O)O)cc1] | N | CH | 1 |
| 2086 | —S(═O)₂CH₃ | 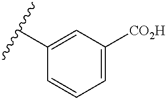 [SMILES: *c1cccc(C(=O)O)c1] | N | CH | 1 |
| 2087 | —S(═O)₂CH₃ | 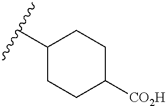 [SMILES: *C1CCC(C(=O)O)CC1] | N | CH | 1 |
| 2088 | —S(═O)₂CH₃ | 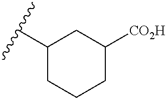 [SMILES: *C1CCCC(C(=O)O)C1] | N | CH | 1 |
| 2089 | —S(═O)₂CH₃ | 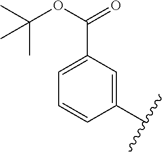 [SMILES: *c1cccc(C(=O)OC(C)(C)C)c1] | N | CH | 1 |
| 2090 | —S(═O)₂CH₃ | 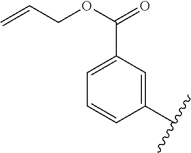 [SMILES: *c1cccc(C(=O)OCC=C)c1] | N | CH | 1 |
| 2091 | —C(═O)CH₃ | 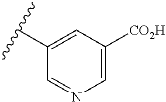 [SMILES: *c1cncc(C(=O)O)c1] | N | CH | 1 |
| 2092 | —C(═O)CH₃ | 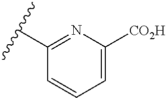 [SMILES: *c1cccc(C(=O)O)n1] | N | CH | 1 |
| 2093 | —C(═O)CH₃ | 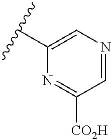 [SMILES: *c1cncc(C(=O)O)n1] | N | CH | 1 |
| 2094 | —C(═O)CH₃ | 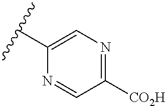 [SMILES: *c1cnc(C(=O)O)cn1] | N | CH | 1 |
| 2095 | —C(═O)CH₃ | 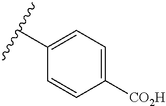 [SMILES: *c1ccc(C(=O)O)cc1] | N | CH | 1 |
| 2096 | —C(═O)CH₃ | 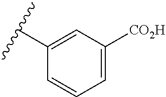 [SMILES: *c1cccc(C(=O)O)c1] | N | CH | 1 |
| 2097 | —C(═O)CH₃ | 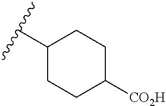 [SMILES: *C1CCC(C(=O)O)CC1] | N | CH | 1 |
| 2098 | —C(═O)CH₃ | 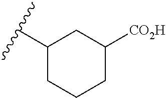 [SMILES: *C1CCCC(C(=O)O)C1] | N | CH | 1 |
| 2099 | —C(═O)CH₃ | 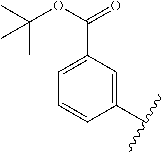 [SMILES: *c1cccc(C(=O)OC(C)(C)C)c1] | N | CH | 1 |
| 2100 | —C(═O)CH₃ | 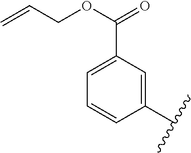 [SMILES: *c1cccc(C(=O)OCC=C)c1] | N | CH | 1 |
| 2101 | 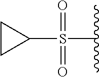 [SMILES: *S(=O)(=O)C1CC1] | 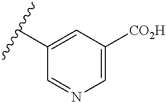 [SMILES: *c1cncc(C(=O)O)c1] | N | CH | 1 |
| 2102 | 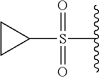 [SMILES: *S(=O)(=O)C1CC1] | 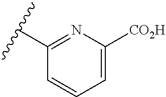 [SMILES: *c1cccc(C(=O)O)n1] | N | CH | 1 |
| 2103 | 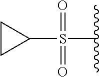 [SMILES: *S(=O)(=O)C1CC1] | 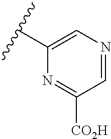 [SMILES: *c1cncc(C(=O)O)n1] | N | CH | 1 |
| 2104 | 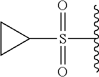 [SMILES: *S(=O)(=O)C1CC1] | 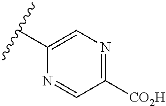 [SMILES: *c1cnc(C(=O)O)cn1] | N | CH | 1 |
| 2105 | 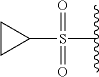 [SMILES: *S(=O)(=O)C1CC1] | 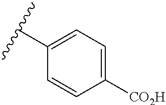 [SMILES: *c1ccc(C(=O)O)cc1] | N | CH | 1 |
| 2106 | 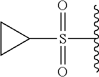 [SMILES: *S(=O)(=O)C1CC1] | 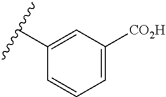 [SMILES: *c1cccc(C(=O)O)c1] | N | CH | 1 |
| 2107 | 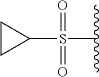 [SMILES: *S(=O)(=O)C1CC1] | 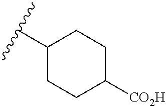 [SMILES: *C1CCC(C(=O)O)CC1] | N | CH | 1 |
| 2108 | 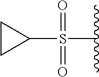 [SMILES: *S(=O)(=O)C1CC1] | 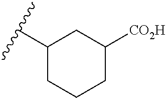 [SMILES: *C1CCCC(C(=O)O)C1] | N | CH | 1 |
| 2109 | 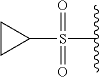 [SMILES: *S(=O)(=O)C1CC1] | 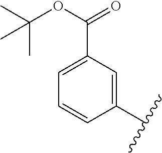 [SMILES: *c1cccc(C(=O)OC(C)(C)C)c1] | N | CH | 1 |
| 2110 | 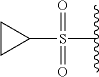 [SMILES: *S(=O)(=O)C1CC1] | 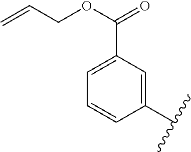 [SMILES: *c1cccc(C(=O)OCC=C)c1] | N | CH | 1 |
| 2111 | H | 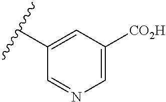 [SMILES: *c1cncc(C(=O)O)c1] | N | CH | 1 |
| 2112 | H | 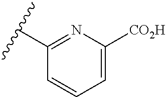 [SMILES: *c1cccc(C(=O)O)n1] | N | CH | 1 |
| 2113 | H | 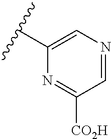 [SMILES: *c1cncc(C(=O)O)n1] | N | CH | 1 |
| 2114 | H | 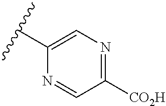 [SMILES: *c1cnc(C(=O)O)cn1] | N | CH | 1 |
| 2115 | H | 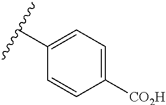 [SMILES: *c1ccc(C(=O)O)cc1] | N | CH | 1 |
| 2116 | H | 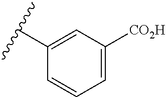 [SMILES: *c1cccc(C(=O)O)c1] | N | CH | 1 |
| 2117 | H | 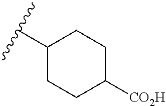 [SMILES: *C1CCC(C(=O)O)CC1] | N | CH | 1 |
| 2118 | H | 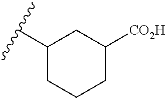 [SMILES: *C1CCCC(C(=O)O)C1] | N | CH | 1 |
| 2119 | H | 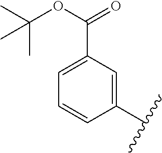 [SMILES: *c1cccc(C(=O)OC(C)(C)C)c1] | N | CH | 1 |
| 2120 | H | 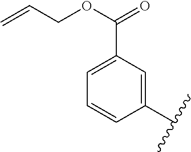 [SMILES: *c1cccc(C(=O)OCC=C)c1] | N | CH | 1 |
| | |
|---|---|
| | Formula (IIc) |
| 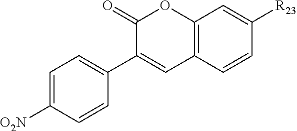 | |
| Compound # | R₂₃ | R₂₁ | p |
|---|---|---|---|
| 2121 | —OH | N/A | N/A |
| 2122 | 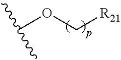 | 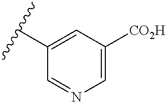 [SMILES: *c1cncc(C(=O)O)c1] | 1 |
| 2123 | 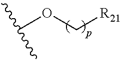 | 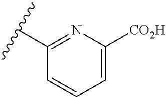 [SMILES: *c1cccc(C(=O)O)n1] | 1 |
| 2124 | 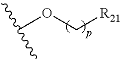 | 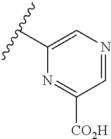 [SMILES: *c1cncc(C(=O)O)n1] | 1 |
| 2125 | 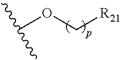 | 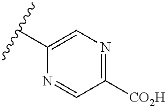 [SMILES: *c1cnc(C(=O)O)cn1] | 1 |
| 2126 | 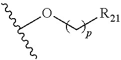 | 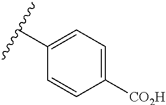 [SMILES: *c1ccc(C(=O)O)cc1] | 1 |
| 2127 | 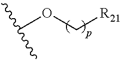 | 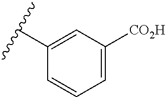 [SMILES: *c1cccc(C(=O)O)c1] | 1 |
| 2128 | 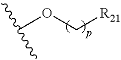 | 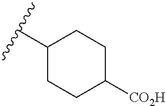 [SMILES: *C1CCC(C(=O)O)CC1] | 1 |
| 2129 | 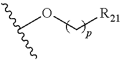 | 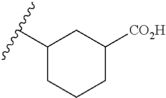 [SMILES: *C1CCCC(C(=O)O)C1] | 1 |
| 2130 | 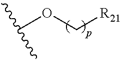 | 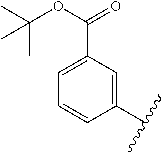 [SMILES: *c1cccc(C(=O)OC(C)(C)C)c1] | 1 |
| 2131 | 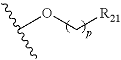 | 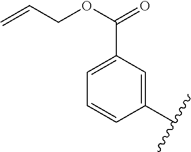 [SMILES: *c1cccc(C(=O)OCC=C)c1] | 1 |

22. The compound of claim 21, wherein the compound of Formula (I) is a compound of formula 2046, or a pharmaceutically acceptable salt, solvate, hydrate, cocrystal, or prodrug thereof:

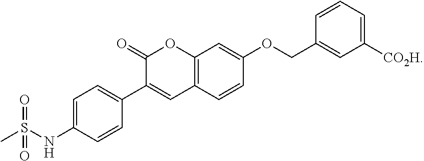 [SMILES: CS(=O)(=O)Nc1ccc(-c2cc3ccc(OCc4cccc(C(=O)O)c4)cc3oc2=O)cc1]

23. The compound of claim 1, wherein:
\textcircled{A} is

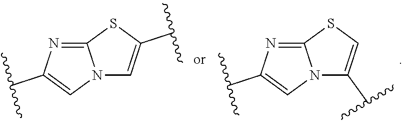 [SMILES: *c1cn2cc(*)sc2n1 ; *c1cn2c(*)csc2n1]

24. The compound of claim 23, wherein the compound of Formula (I) is a compound of Formula (IIIa) Formula (IIIb), Formula (IIIc), or a pharmaceutically acceptable salt, solvate, hydrate, cocrystal, or prodrug thereof: 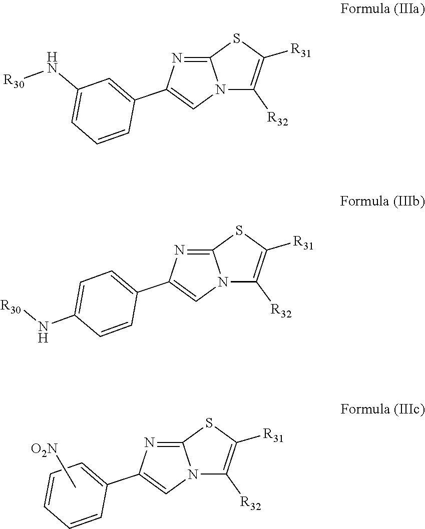
wherein
R₃₀ is selected from H, —S(═O)₂R₃₄, and —C(═O)₂R₃₄;
R₃₁ and R₃₂ are each independently selected from H, alkyl, —OH, —C(═O)H, —C(═O)OH, —C(═O)OR₃₃, and —C(═O)NH₂;
R₃₃ is alkyl or alkenyl; and
R₃₄ is selected from optionally substituted alkyl and optionally substituted cycloalkyl.

25. The compound of claim 22 or 24, wherein R₃₀ is selected from H, —S(═O)₂CH₃, —C(═O)CH₃, and

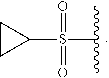 [SMILES: *S(=O)(=O)C1CC1]

26. The compound claim 24 or 25, wherein one of R₃₁ or R₃₂ is —OH, —C(═O)H, —C(═O)OH, —C(═O)OR₃₃, and —C(═O)NH₂.

27. The compound of claim 24, wherein the compound is of any one of formulas 3001 to 3073, or a pharmaceutically acceptable salt, solvate, hydrate, cocrystal, or prodrug thereof:

| | |
|---|---|
| | Formula (IIIa) |
| 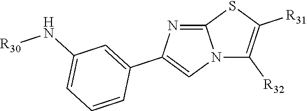 | |
| Com- | | | |
|---|---|---|---|
| pound | | | |
| # | R₃₀ | R₃₁ | R₃₁ |
| 3001 | —S(═O)₂CH₃ | —C(═O)OH | H |
| 3002 | —S(═O)₂CH₃ | —C(═O)OCH₂CH₃ | H |
| 3003 | —S(═O)₂CH₃ | H | —C(═O)OH |
| 3004 | —S(═O)₂CH₃ | H | —C(═O)OCH₂CH₃ |
| 3005 | —S(═O)₂CH₃ | —C(═O)OH | —CH₃ |
| 3006 | —S(═O)₂CH₃ | —C(═O)OCH₂CH₃ | —CH₃ |
| 3007 | —S(═O)₂CH₃ | —CH₃ | —C(═O)OH |
| 3008 | —S(═O)₂CH₃ | —CH₃ | —C(═O)OCH₂CH₃ |
| 3009 | —C(═O)CH₃ | —C(═O)OH | H |
| 3010 | —C(═O)CH₃ | —C(═O)OCH₂CH₃ | H |
| 3011 | —C(═O)CH₃ | H | —C(═O)OH |
| 3012 | —C(═O)CH₃ | H | —C(═O)OCH₂CH₃ |
| 3013 | —C(═O)CH₃ | —C(═O)OH | —CH₃ |
| 3014 | —C(═O)CH₃ | —C(═O)OCH₂CH₃ | —CH₃ |
| 3015 | —C(═O)CH₃ | —CH₃ | —C(═O)OH |
| 3016 | —C(═O)CH₃ | —CH₃ | —C(═O)OCH₂CH₃ |
| 3017 | 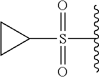 [SMILES: *S(=O)(=O)C1CC1] | —C(═O)OH | H |
| 3018 | 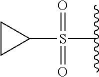 [SMILES: *S(=O)(=O)C1CC1] | —C(═O)OCH₂CH₃ | H |
| 3019 | 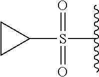 [SMILES: *S(=O)(=O)C1CC1] | H | —C(═O)OH |
| 3020 | 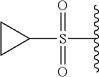 [SMILES: *S(=O)(=O)C1CC1] | H | —C(═O)OCH₂CH₃ |
| 3021 | 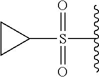 [SMILES: *S(=O)(=O)C1CC1] | —C(═O)OH | —CH₃ |
| 3022 | 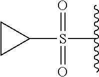 [SMILES: *S(=O)(=O)C1CC1] | —C(═O)OCH₂CH₃ | —CH₃ |
| 3023 | 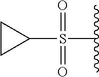 [SMILES: *S(=O)(=O)C1CC1] | —CH₃ | —C(═O)OH |
| 3024 | 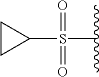 [SMILES: *S(=O)(=O)C1CC1] | —CH₃ | —C(═O)OCH₂CH₃ |
| 3025 | H | —C(═O)OH | H |
| 3026 | H | —C(═O)OCH₂CH₃ | H |
| 3027 | H | H | —C(═O)OH |
| 3028 | H | H | —C(═O)OCH₂CH₃ |
| 3029 | H | —C(═O)OH | —CH₃ |
| 3030 | H | —C(═O)OCH₂CH₃ | —CH₃ |
| 3031 | H | —CH₃ | —C(═O)OH |
| 3032 | H | —CH₃ | —C(═O)OCH₂CH₃ |
| | |
|---|---|
| | Formula (IIIb) |
| 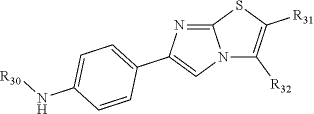 | |
| Com- | | | |
|---|---|---|---|
| pound | | | |
| # | R₃₀ | R₃₁ | R₃₂ |
| 3033 | —S(═O)₂CH₃ | —C(═O)OH | H |
| 3034 | —S(═O)₂CH₃ | —C(═O)OCH₂CH₃ | H |
| 3035 | —S(═O)₂CH₃ | H | —C(═O)OH |
| 3036 | —S(═O)₂CH₃ | H | —C(═O)OCH₂CH₃ |
| 3037 | —S(═O)₂CH₃ | —C(═O)OH | —CH₃ |
| 3038 | —S(═O)₂CH₃ | —C(═O)OCH₂CH₃ | —CH₃ |
| 3039 | —S(═O)₂CH₃ | —CH₃ | —C(═O)OH |
| 3040 | —S(═O)₂CH₃ | —CH₃ | —C(═O)OCH₂CH₃ |
| 3041 | —C(═O)CH₃ | —C(═O)OH | H |
| 3042 | —C(═O)CH₃ | —C(═O)OCH₂CH₃ | H |
| 3043 | —C(═O)CH₃ | H | —C(═O)OH |
| 3044 | —C(═O)CH₃ | H | —C(═O)OCH₂CH₃ |
| 3045 | —C(═O)CH₃ | —C(═O)OH | —CH₃ |
| 3046 | —C(═O)CH₃ | —C(═O)OCH₂CH₃ | —CH₃ |
| 3047 | —C(═O)CH₃ | —CH₃ | —C(═O)OH |
| 3048 | —C(═O)CH₃ | —CH₃ | —C(═O)OCH₂CH₃ |
| 3049 | 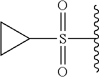 [SMILES: *S(=O)(=O)C1CC1] | —C(═O)OH | H |
| 3050 | 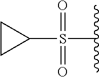 [SMILES: *S(=O)(=O)C1CC1] | —C(═O)OCH₂CH₃ | H |
| 3051 | 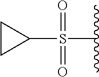 [SMILES: *S(=O)(=O)C1CC1] | H | —C(═O)OH |
| 3052 | 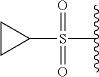 [SMILES: *S(=O)(=O)C1CC1] | H | —C(═O)OCH₂CH₃ |
| 3053 | 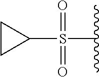 [SMILES: *S(=O)(=O)C1CC1] | —C(═O)OH | —CH₃ |
| 3054 | 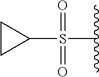 [SMILES: *S(=O)(=O)C1CC1] | —C(═O)OCH₂CH₃ | —CH₃ |
| 3055 | 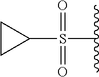 [SMILES: *S(=O)(=O)C1CC1] | —CH₃ | —C(═O)OH |
| 3056 | 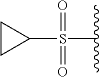 [SMILES: *S(=O)(=O)C1CC1] | —CH₃ | —C(═O)OCH₂CH₃ |
| 3057 | H | —C(═O)OH | H |
| 3058 | H | —C(═O)OCH₂CH₃ | H |
| 3059 | H | H | —C(═O)OH |
| 3060 | H | H | —C(═O)OCH₂CH₃ |
| 3061 | H | —C(═O)OH | —CH₃ |
| 3062 | H | —C(═O)OCH₂CH₃ | —CH₃ |
| 3063 | H | —CH₃ | —C(═O)OH |
| 3064 | H | —CH₃ | —C(═O)OCH₂CH₃ |
| | |
|---|---|
| | Formula (IIIc) |
| 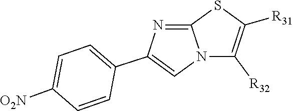 | |
| | R₃₁ | R₃₂ |
|---|---|---|
| 3066 | —C(═O)OH | H |
| 3067 | —C(═O)OCH₂CH₃ | H |
| 3068 | H | —C(═O)OH |
| 3069 | H | —C(═O)OCH₂CH₃ |
| 3070 | —C(═O)OH | —CH₃ |
| 3071 | —C(═O)OCH₂CH₃ | —CH₃ |
| 3072 | —CH₃ | —C(═O)OH |
| 3073 | —CH₃ | —C(═O)OCH₂CH₃ |

28. The compound of claim 27, wherein the compound of Formula (I) is a compound of formula 3035, or a pharmaceutically acceptable salt, solvate, hydrate, cocrystal, or prodrug thereof:

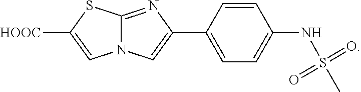

29. The compound of claim 25, wherein the compound of Formula (I) is a compound of formula 3037, or a pharmaceutically acceptable salt, solvate, hydrate, cocrystal, or prodrug thereof:

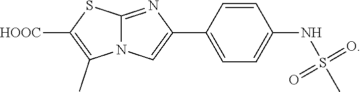

30. The compound of any one of claims 1-29, wherein the compound inhibits alcohol dehydrogenase-2 (ALDH2) protein.

31. The compound of any one of claims 1-30, wherein the compound inhibits liver-specific alcohol dehydrogenase-2 (ALDH2) protein.

32. The compound of any one of claims 1-31, wherein the compound can be actively taken up as a substrate for a liver-specific organic anion transporting polypeptide (OATP) transporter.

33. A pharmaceutical composition comprising a compound of any one of claims 1-32 or a pharmaceutically acceptable salt, solvate, hydrate, cocrystal, or prodrug thereof, and a pharmaceutically acceptable carrier or excipient.

34. A method of treating a condition by inhibiting alcohol dehydrogenase-2 (ALDH2) protein activity in a patient in need of said treatment, the method comprising administering to the patient a therapeutically effective amount of a compound of any of claims 1 to 32, or a pharmaceutically acceptable salt, solvate, hydrate, cocrystal, or prodrug thereof, or a therapeutically effective amount of the pharmaceutical composition of claim 33.

35. The method of claim 34, wherein the compound inhibits liver-specific ALDH2 protein.

36. The method of claim 34 or 35, wherein the condition is an alcohol use disorder.
